(19) United States
(12) Patent Application Publication
(10) Pub. No.: US 2023/0039560 A1
(43) Pub. Date: Feb. 9, 2023

(54) Hypodermic Interface Assembly

(71) Applicant: Neogen Corporation, Lansing, MI (US)

(72) Inventors: Randall Marc Bachtel, Lawrenceville, GA (US); Bradley E. Galbreath, Lexington, KY (US); Gregory Steven Hastings, Lexington, KY (US); Dexter Edmund Jacobs, Austin, TX (US); Taylor Kopacka Leigh, Milton, GA (US); Jason Lye, Atlanta, GA (US); Peter Wyndham Shipp, JR., Woodstock, GA (US); Michael Trovato, Vancouver, WA (US); Nicholas John Wagner, Lexington, KY (US)

(21) Appl. No.: 17/968,327

(22) Filed: Oct. 18, 2022

Related U.S. Application Data
(63) Continuation-in-part of application No. 16/947,305, filed on Jul. 28, 2020.

(51) Int. Cl. A61M 5/32

(52) U.S. Cl. CPC A61M 5/3202

(57) ABSTRACT

A hypodermic interface assembly is disclosed. The hypodermic interface assembly includes a hub; a cannula; a core member; and a cannula carrier. The core member is non-removably connected to the cannula. The cannula carrier is non-removably connected to the attachment member. The cannula carrier is controllably separable from the hub. A subassembly is also disclosed. The subassembly includes a hub; a cannula; and a core member. A proximal end surface of the core member is arranged at a predetermined hub-core distance away from a distal end surface of the hub.

### CROSS REFERENCE TO RELATED APPLICATION

This patent application is a continuation-in-part of U.S. application Ser. No. 16/947,305, filed Jul. 28, 2020. The disclosure of this prior application is considered part of this disclosure and is hereby incorporated by reference in its entirety.

### TECHNICAL FIELD

The disclosure relates generally to hypodermic interface assemblies.

### BACKGROUND

This section provides background information related to the present disclosure and is not necessarily prior art.

While known hypodermic interface assemblies have proven to be acceptable for various applications, such hypodermic interface assemblies are nevertheless susceptible to improvements that may enhance their overall performance and cost. Therefore, a need exists to develop hypodermic interface assemblies that advance the art.

### SUMMARY

This section provides a general summary of the disclosure, and is not a comprehensive disclosure of its full scope or all of its features.

In one aspect, the invention provides a hypodermic interface assembly that includes: a hub; a cannula; a core member; and a cannula carrier, wherein the core member is non-removably connected to the cannula, the cannula carrier is non-removably connected to the core member, and the cannula carrier is controllably separable from the hub. Further, the core member may have a body; and the body may be defined by a proximal end surface, a distal end surface, and an outer surface extending between the proximal end surface and the distal end surface, wherein the body includes a cannula-receiving-passage defined by an inner surface that extends through the body of the core member.

In another aspect of the invention, the distal end surface of the body of the core member may extend distally beyond a distal end surface of the cannula carrier. Or the core member may include a cannula-carrier-engaging portion and the cannula carrier may be configured to receive the cannula-carrier-engaging portion; and further, the cannula-carrier-engaging portion of the core member may be non-removably connected to the cannula carrier; and, the cannula-carrier-engaging portion of the core member may be friction-fit connected to the cannula carrier.

In a further aspect of the invention, the cannula is disposed within: a hub passage extending through the hub; the cannula-receiving-passage of the core member; and a cannula carrier passage extending through the cannula carrier. Further, a first portion of an outer surface of the cannula may be arranged in a spaced-apart relationship with respect to a surface portion that defines the cannula carrier passage of the cannula carrier, and a second portion of the outer surface of the cannula may be disposed adjacent and non-removably-connected to a surface portion defining the cannula-receiving-passage of the core member.

In another aspect of the invention, the cannula carrier includes a head portion and at least one leg portion, and a proximal end surface of the at least one leg portion is disposed adjacent an outer shoulder surface portion of an outer surface of the hub.

In a further aspect of the invention, the hub is defined by a distal end surface; the core member is defined by a body having a proximal end surface; a portion of the cannula has a length that extends between the distal end surface of the hub and the proximal end surface of the core member and defines a hub-core distance; and the hub-core distance is configured to permit the cannula to bend or break at a region bound by the distal end surface of the hub and the proximal end surface of the core member when the hypodermic interface assembly is subjected to one or more radial forces.

In yet another aspect of the invention, the cannula is arranged relative the hub and the core member such that a first portion of the cannula is arranged within the hub, a second portion of the cannula extends between a distal end surface of the hub and a proximal end surface of the core member, a third portion of the cannula is arranged within the core member, and a fourth portion of the cannula extends beyond a distal end surface of the core member. Further, the second portion of the cannula may be configured to permit the cannula to bend or break at a region bound by the second portion of the cannula when the hypodermic interface assembly is subjected to one or more radial forces.

In another aspect, the invention provides a subassembly of a hypodermic interface assembly including: a hub having a distal end surface; a cannula having an outer surface that is non-removably connected to the hub, wherein the cannula extends distally away from the distal end surface of the hub; and a core member having a body defined by a proximal end surface and a distal end surface, wherein the proximal end surface of the body is arranged at a hub-core distance away from a distal end surface of the hub. The body may include an inner surface defining a cannula-receiving-passage that contains a portion of a length of the cannula, wherein the cannula-receiving-passage defines a passage diameter, and wherein the inner surface is arrangeable in one of an inner surface preconfigured state and an inner surface deformed state.

In a further aspect of the subassembly of a hypodermic interface assembly, when the inner surface is arranged in the inner surface preconfigured state, the cannula-receiving-passage may be a cannula-receiving-passage and the passage diameter may be greater than an outer diameter of the cannula such that the outer surface of the cannula is not joined to the inner surface of the body of the core member. Further, when the inner surface is arranged in the inner surface deformed state, the cannula-receiving-passage may be a cannula-receiving-passage such that the passage diameter may be deformed to define a deformed diameter that may be approximately the same as but slightly greater than the outer diameter of the cannula whereby the outer surface of the cannula is non-removably connected to a deformed inner surface of the inner surface extending along at least a portion of the length of the body of the core member and defines the deformed diameter. Even further, the passage diameter of the cannula-receiving-passage may be constant along the length of the body of the core member, the deformed inner surface of the inner surface may be a portion of the inner surface of the body of the core member whereby the deformed diameter defines the passage diameter to be non-constant along the length of the body of the core member.

In another aspect of the subassembly of a hypodermic interface assembly, the body of the core member may include an outer surface arrangeable in one of: an outer surface preconfigured state whereby the outer surface defines a preconfigured dimension; and an outer surface deformed state whereby the outer surface defines a preconfigured-and-subsequently-deformed dimension whereby the portion of the length of the body of the core member defines a deformed outer surface of the outer surface that defines the preconfigured-and-subsequently-deformed dimension to be different from the preconfigured dimension. Further, the proximal end surface may define a proximal opening of the body that permits proximal access to the cannula-receiving-passage, and the distal end surface may define a distal opening of the body that permits distal access to the cannula-receiving-passage. Even further, a proximal portion of the cannula may extend through the proximal opening of the body, and a distal portion of the cannula may extend through the proximal opening of the body.

The details of one or more implementations of the disclosure are set forth in the accompanying drawings and the description below. Other aspects, features, and advantages will be apparent from the description and drawings, and from the claims.

### DESCRIPTION OF DRAWINGS

The drawings described herein are for illustrative purposes only of selected configurations and not all possible implementations, and are not intended to limit the scope of the present disclosure.

FIG. 1 is an exploded perspective view of an exemplary hypodermic interface assembly.

FIG. 2 is a perspective view of an exemplary cannula of the hypodermic interface assembly of FIG. 1.

FIG. 3 is a front perspective view of an exemplary hub of the hypodermic interface assembly of FIG. 1.

FIG. 4 is a rear perspective view of the hub of FIG. 2.

FIG. 5 is another front perspective view of the hub of FIG. 2.

FIG. 6 is a side view of the hub of FIG. 2.

FIG. 7 is a cross-sectional view of the hub according to line 7-7 of FIG. 6.

FIG. 8A is another side view of the hub of FIG. 2.

FIG. 8B is a top view of the hub according to arrow 8B of FIG. 8A.

FIG. 9A is another side view of the hub of FIG. 2.

FIG. 9B is a top view of the hub according to arrow 9B of FIG. 9A.

FIG. 10 is a bottom view of the hub according to arrow 10 of FIG. 8A or 9A.

FIG. 11A is a cross-sectional view of a sub-assembly of the hypodermic interface assembly arranged in a first partially assembled state according to line 11A-11A of FIG. 1.

FIG. 11B is a cross-sectional view of a partially assembled hypodermic interface sub-assembly of FIG. 11A arranged in a second partially assembled state.

FIG. 11C is a cross-sectional view of an assembled hypodermic interface sub-assembly of FIG. 11B according to line 11′-11′ of any of FIG. 12.

FIG. 12 is an assembled front perspective view of the hypodermic interface sub-assembly of FIG. 11C.

FIG. 13 is a top view of the hypodermic interface sub-assembly according to arrow 13 of FIG. 12.

FIG. 14 is a bottom view of the hypodermic interface sub-assembly according to arrow 14 of FIG. 12.

FIG. 15 is an assembled rear perspective view of the hypodermic interface sub-assembly of FIG. 11C.

FIG. 16 is another assembled front perspective view of the hypodermic interface sub-assembly of FIG. 11C.

FIG. 17 is a side view of the hypodermic interface sub-assembly of FIG. 11C.

FIG. 18 is a front perspective view of an exemplary cannula carrier of the hypodermic interface assembly of FIG. 1.

FIG. 19 is another front perspective view of an exemplary cannula carrier of FIG. 18 rotated 90°.

FIG. 20 is another front perspective view of an exemplary cannula carrier of FIG. 19 rotated 90°.

FIG. 21 is another front perspective view of an exemplary cannula carrier of FIG. 20 rotated 90°.

FIG. 22 is a rear perspective view of the cannula carrier of FIG. 18.

FIG. 23 is another front perspective view of the cannula carrier of FIG. 18.

FIG. 24 is a top view of the cannula carrier according to arrow 24 of any of FIGS. 18-23.

FIG. 25 is a bottom view of the cannula carrier according to arrow 25 of any of FIGS. 18-23.

FIG. 26A is a side view of the cannula carrier of FIG. 18.

FIG. 26B is a side view of the cannula carrier of FIG. 26A rotated 45°.

FIG. 26C is a side view of the cannula carrier of FIG. 26B rotated 45°.

FIG. 26D is a side view of the cannula carrier of FIG. 26C rotated 45°.

FIG. 26E is a side view of the cannula carrier of FIG. 26D rotated 45°.

FIG. 26F is a side view of the cannula carrier of FIG. 26E rotated 45°.

FIG. 26G is a side view of the cannula carrier of FIG. 26F rotated 45°.

FIG. 26H is a side view of the cannula carrier of FIG. 26G rotated 45°.

FIG. 27A is a bottom view of the cannula carrier corresponding to FIG. 26A.

FIG. 27B is a bottom view of the cannula carrier corresponding to FIG. 26B.

FIG. 27C is a bottom view of the cannula carrier corresponding to FIG. 26C.

FIG. 27D is a bottom view of the cannula carrier corresponding to FIG. 26D.

FIG. 27E is a bottom view of the cannula carrier corresponding to FIG. 26E.

FIG. 27F is a bottom view of the cannula carrier corresponding to FIG. 26F.

FIG. 27G is a bottom view of the cannula carrier corresponding to FIG. 26G.

FIG. 27H is a bottom view of the cannula carrier corresponding to FIG. 26H.

FIG. 28 is an exploded front perspective view of portions of the hypodermic interface assembly of FIG. 1 including the cannula of FIG. 2, the hub of FIGS. 3-10, and the cannula carrier of FIGS. 18-27H.

FIG. 29 is a cross-sectional view according to line 29-29 of FIG. 28.

FIG. 30 is another cross-sectional view according to FIG. 29 illustrating the cannula carrier disposed about the hypodermic interface sub-assembly of FIGS. 11C-17.

FIG. 31 is another cross-sectional view according to FIG. 30 illustrating an adhesive being metered into a passage portion of the cannula carrier for non-removably-joining the cannula carrier to the cannula of the hypodermic interface sub-assembly.

FIG. 32 is another cross-sectional view according to FIG. 31 illustrating light that cures the adhesive that non-removably-joins the cannula carrier to the cannula of the hypodermic interface sub-assembly.

FIG. 33 is an assembled rear perspective view of the hypodermic interface assembly of FIG. 1.

FIG. 34 is another assembled front perspective view of the hypodermic interface assembly of FIG. 1.

FIG. 35A is perspective cross-sectional view according to line 35-35 of the front perspective view of the hypodermic interface assembly of FIG. 34 that is arranged in an at-rest orientation.

FIG. 35B is side cross-sectional view of the assembled hypodermic interface assembly according to arrow 35B of FIG. 35A.

FIG. 36A is another perspective cross-sectional view according to the front perspective view of the hypodermic interface assembly of FIG. 35A that is arranged in a biased orientation.

FIG. 36B is side cross-sectional view of the assembled hypodermic interface assembly according to arrow 36B of FIG. 36A.

FIG. 37A is another perspective cross-sectional view according to the front perspective view of the hypodermic interface assembly of FIG. 35A that is arranged in a separated orientation.

FIG. 37B is side cross-sectional view of the assembled hypodermic interface assembly according to arrow 37B of FIG. 37A.

FIG. 38A is an enlarged view of the assembled hypodermic interface assembly according to line 38A of FIG. 35B.

FIG. 38B is a cross-sectional side view of the assembled hypodermic interface assembly of FIG. 38A.

FIG. 38C is a cross-sectional side view of another assembled hypodermic interface assembly.

FIG. 38D is a cross-sectional side view of another assembled hypodermic interface assembly.

FIG. 38E is a cross-sectional side view of another assembled hypodermic interface assembly.

FIG. 38F is a cross-sectional side view of another assembled hypodermic interface assembly.

FIG. 39A is perspective cross-sectional view according to the side perspective view of the hypodermic interface assembly of FIG. 38C that is arranged in an at-rest orientation.

FIG. 39B is another perspective cross-sectional view according to the front perspective view of the hypodermic interface assembly of FIG. 39A that is arranged in a biased orientation.

FIG. 39C is another perspective cross-sectional view according to the front perspective view of the hypodermic interface assembly of FIG. 39B that is arranged in a separated orientation.

FIG. 40 is a view of a hypodermic interface assembly arranged proximate animalia.

FIG. 41A is a side view of the hypodermic interface assembly and a cross-sectional view of a portion of the animalia of FIG. 40 arranged in a spaced-apart relationship.

FIG. 41B is another side view of the hypodermic interface assembly and another cross-sectional view of a portion of the animalia according to FIG. 41A arranged in a pierced relationship.

FIG. 41C is another side view of the hypodermic interface assembly and another cross-sectional view of a portion of the animalia according to FIG. 41B arranged in a pierced relationship while, optionally, the hypodermic interface assembly is utilized for injecting a fluid into the animalia.

FIG. 41D is another side view of the hypodermic interface assembly and another cross-sectional view of a portion of the animalia according to FIG. 41B arranged in a pierced-and-torqued relationship.

FIG. 41E is another side view of the hypodermic interface assembly and another cross-sectional view of a portion of the animalia according to FIG. 41D arranged in a separated-after-pierced relationship defining a first portion of the hypodermic interface assembly attached to an injection gun and a second portion of the hypodermic interface assembly impaled within flesh of the animalia.

FIG. 41F is another side view of the according to FIG. 41E illustrating a user grasping the second portion of the hypodermic interface assembly that is impaled within flesh of the animalia.

FIG. 41G is another side view of the according to FIG. 41F illustrating the user removing the second portion of the hypodermic interface assembly that was impaled within flesh of the animalia.

FIG. 42 is an exploded perspective view of an exemplary hypodermic interface assembly.

FIG. 43 is an assembled perspective view of the hypodermic interface assembly of FIG. 42.

FIG. 44 is a side view of a core member of the hypodermic interface assembly of FIGS. 42-43.

FIG. 45 is a cross-sectional view of the core member according to line 45-45 of FIG. 44.

FIG. 46 is a bottom view of the core member of FIG. 45.

FIG. 47 is a top view of the core member of FIG. 45.

FIG. 48 is a perspective view of a cannula carrier of the hypodermic interface assembly of FIGS. 42-43.

FIG. 49 is a cross-sectional view of the cannula carrier according to line 49-49 of FIG. 48.

FIG. 50 is a bottom view of the cannula carrier of FIG. 48.

FIG. 51 is a top view of the cannula carrier of FIG. 48.

FIG. 52A is an exploded cross-sectional view of a cannula and a hub defining a first subassembly of the hypodermic interface assembly of FIGS. 42-43.

FIG. 52B is an assembled cross-sectional view of the first subassembly of the hypodermic interface assembly according to FIG. 52A.

FIG. 52C is an exploded cross-sectional view of the first subassembly of FIG. 52B and the core member of FIG. 45 defining a second subassembly of the hypodermic interface assembly of FIGS. 42-43.

FIG. 52D is a partially assembled view of the second subassembly of the hypodermic interface assembly according to FIG. 52C.

FIG. 52E is an assembled view of the second subassembly of the hypodermic interface assembly according to FIG. 52D.

FIG. 52F is an exploded cross-sectional view of the second subassembly of FIG. 52E and the cannula carrier of FIG. 49 defining a third subassembly that defines the hypodermic interface assembly of FIGS. 42-43.

FIG. 52G is a partially assembled view of the third subassembly according to FIG. 52F.

FIG. 52H is an assembled view of the third subassembly according to FIG. 52G.

FIG. 53 is an enlarged view of the hypodermic interface assembly according to line 53 of FIG. 52H.

FIG. 54A is a cross-sectional view of the hypodermic interface assembly of FIG. 52H arranged in an at-rest orientation.

FIG. 54B is a cross-sectional view of the hypodermic interface assembly according to FIG. 54A arranged in a biased orientation.

FIG. 54C, is a cross-sectional view of the hypodermic interface assembly according to FIG. 54A arranged in a separated orientation.

FIG. 55 is a view of the hypodermic interface assembly of FIG. 54A arranged proximate animalia.

FIG. 56A is a side view of the hypodermic interface assembly and a cross-sectional view of a portion of the animalia of FIG. 55 arranged in a spaced-apart relationship.

FIG. 56B is another side view of the hypodermic interface assembly and another cross-sectional view of a portion of the animalia according to FIG. 56A arranged in a pierced relationship.

FIG. 56C is another side view of the hypodermic interface assembly and another cross-sectional view of a portion of the animalia according to FIG. 56B arranged in a pierced relationship while, optionally, the hypodermic interface assembly is utilized for injecting a fluid into the animalia.

FIG. 56D is another side view of the hypodermic interface assembly and another cross-sectional view of a portion of the animalia according to FIG. 56B arranged in a pierced-and-torqued relationship.

FIG. 56E is another side view of the hypodermic interface assembly and another cross-sectional view of a portion of the animalia according to FIG. 56D arranged in a separated-after-pierced relationship defining a first portion of the hypodermic interface assembly attached to an injection gun and a second portion of the hypodermic interface assembly impaled within flesh of the animalia.

FIG. 56F is another side view of the according to FIG. 56E illustrating a user grasping the second portion of the hypodermic interface assembly that is impaled within flesh of the animalia.

FIG. 56G is another side view of the according to FIG. 56F illustrating the user removing the second portion of the hypodermic interface assembly that was impaled within flesh of the animalia.

Corresponding reference numerals indicate corresponding parts throughout the drawings.

### DETAILED DESCRIPTION

Example configurations will now be described more fully with reference to the accompanying drawings. Example configurations are provided so that this disclosure will be thorough, and will fully convey the scope of the disclosure to those of ordinary skill in the art. Specific details are set forth such as examples of specific components, devices, and methods, to provide a thorough understanding of configurations of the present disclosure. It will be apparent to those of ordinary skill in the art that specific details need not be employed, that example configurations may be embodied in many different forms, and that the specific details and the example configurations should not be construed to limit the scope of the disclosure.

The figures illustrate exemplary implementations of hypodermic interface assemblies. Based on the foregoing, it is to be generally understood that the nomenclature used herein is simply for convenience and the terms used herein should be given the broadest meaning by one of ordinary skill in the art.

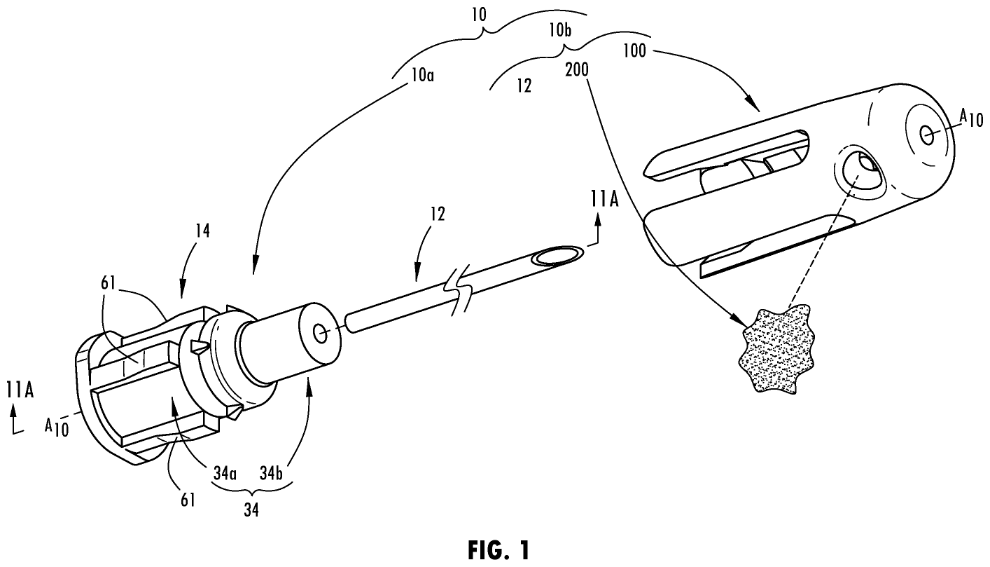

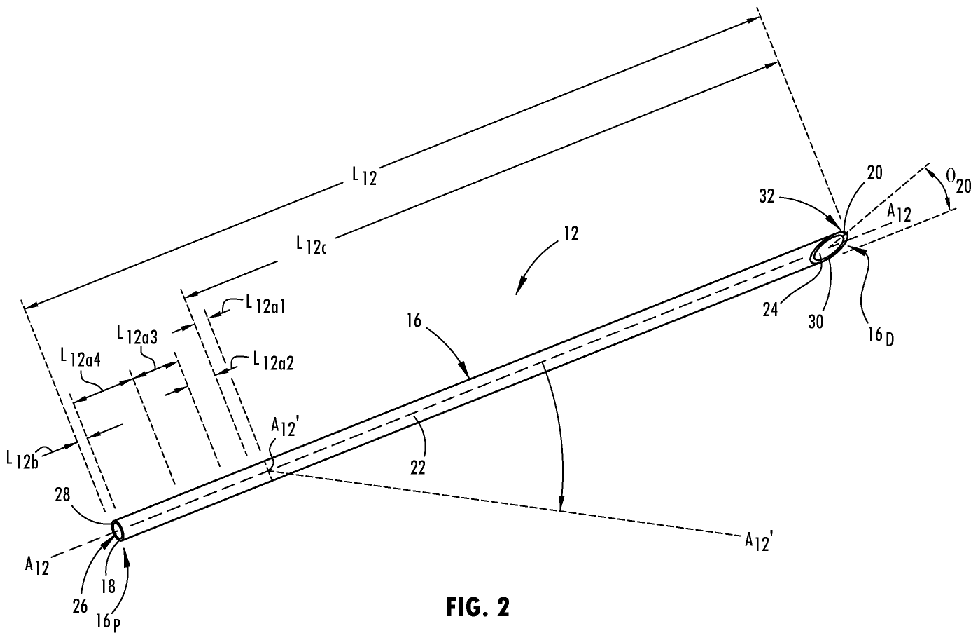

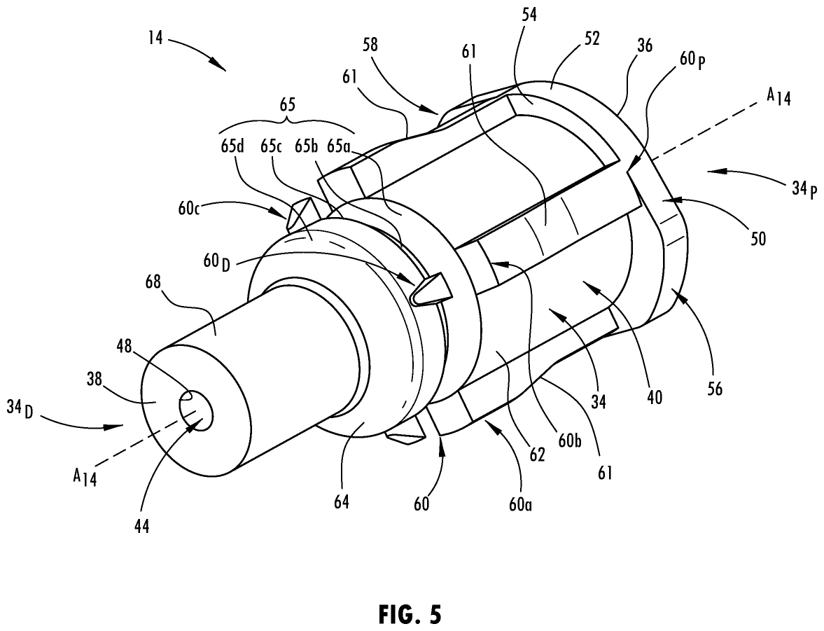

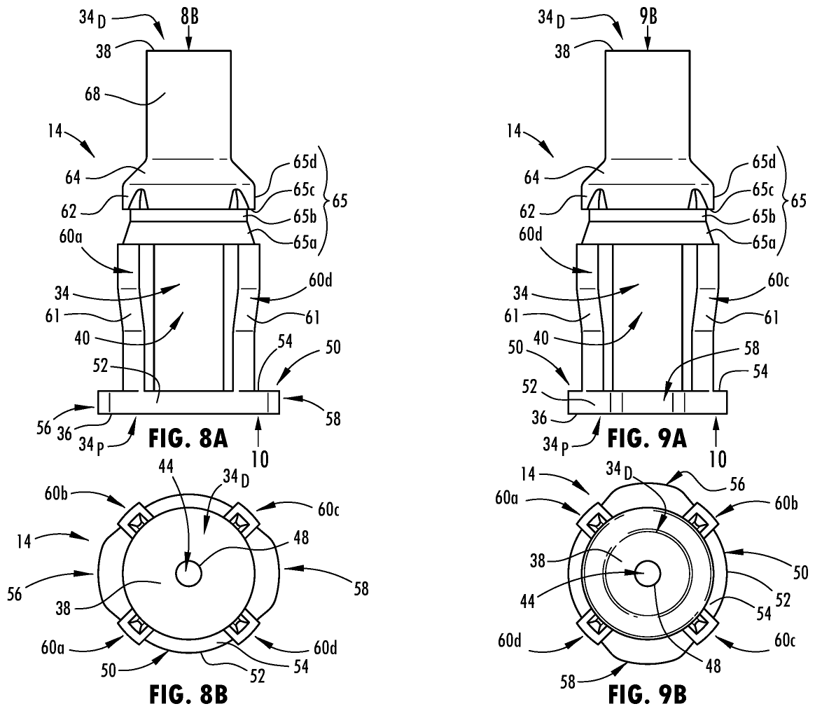

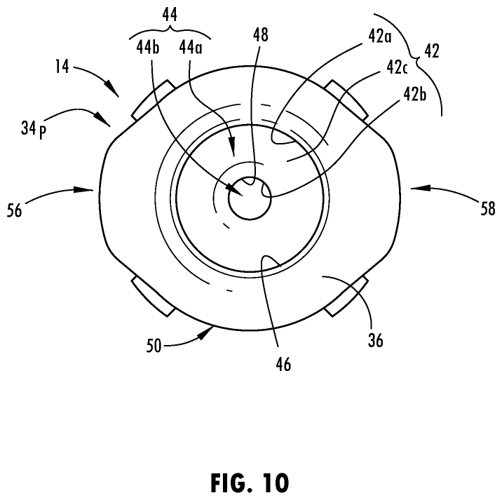

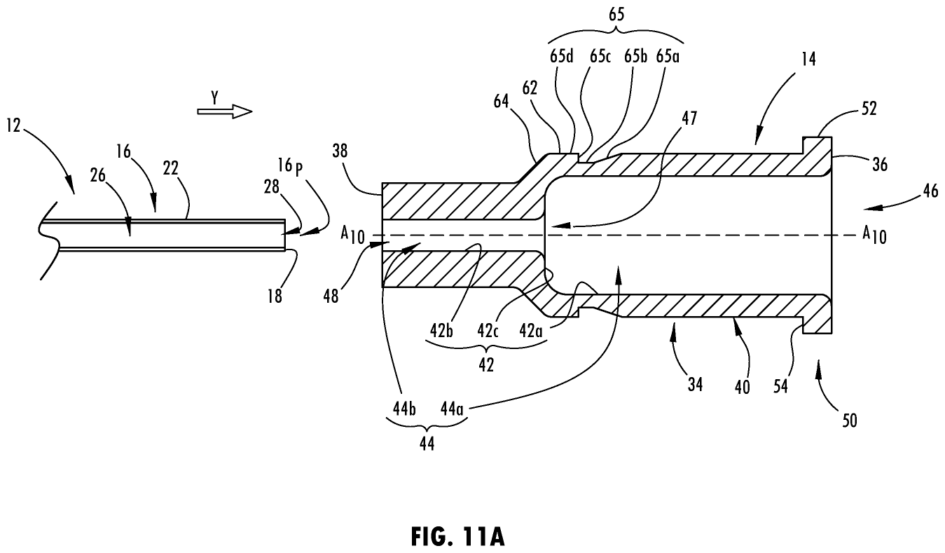

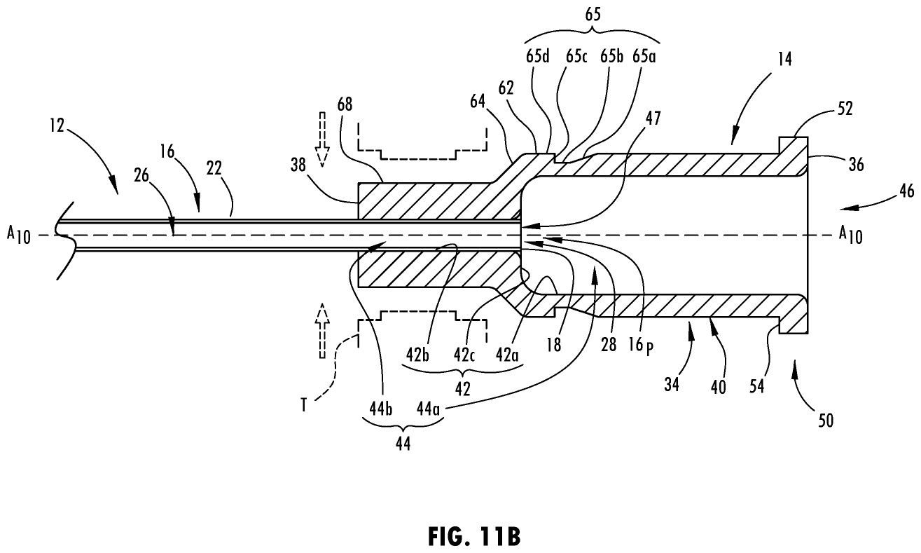

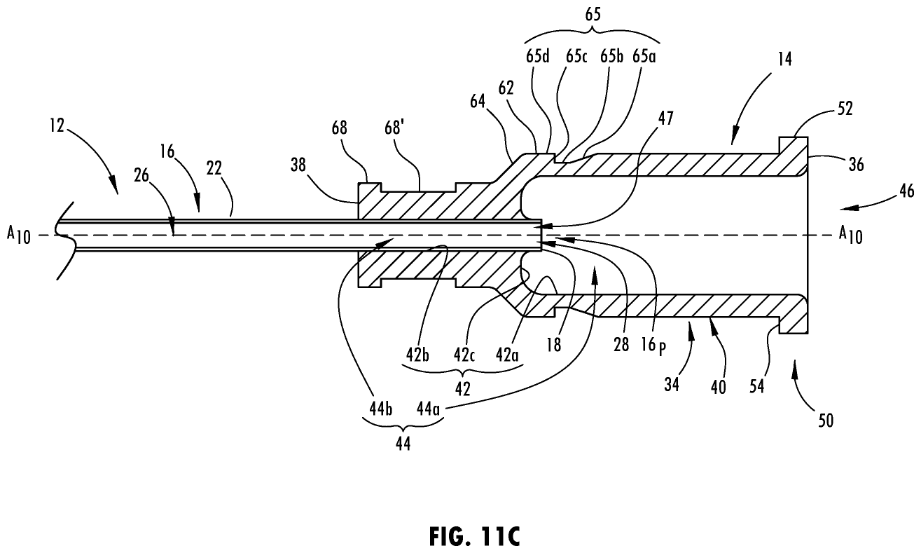

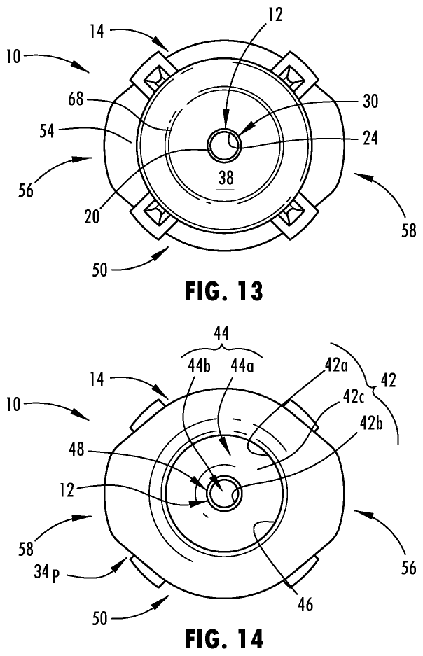

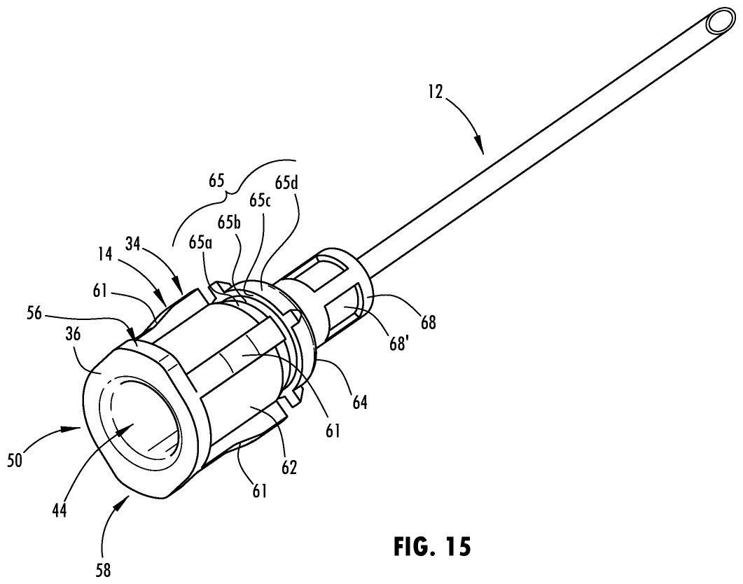

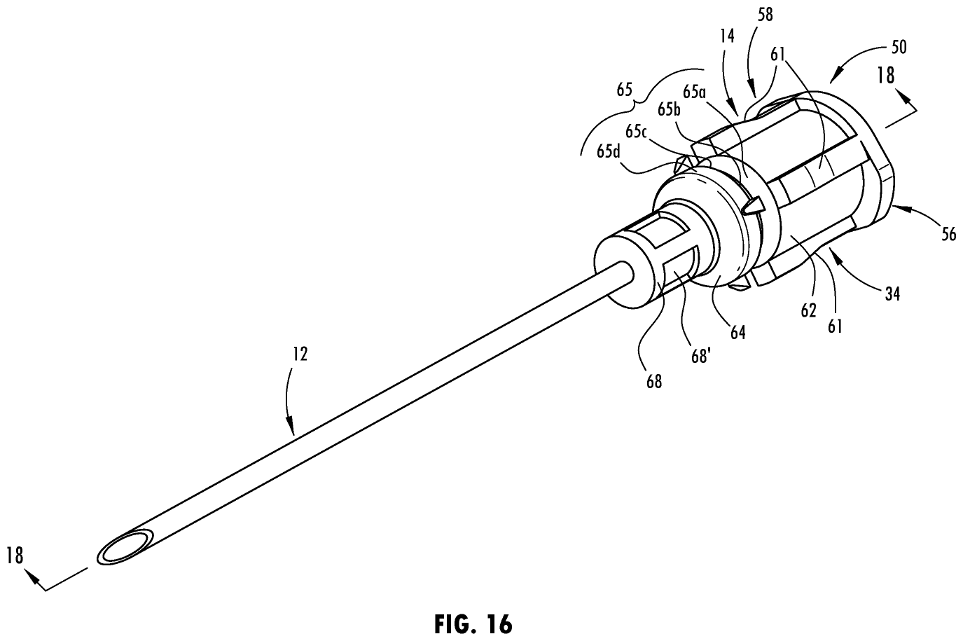

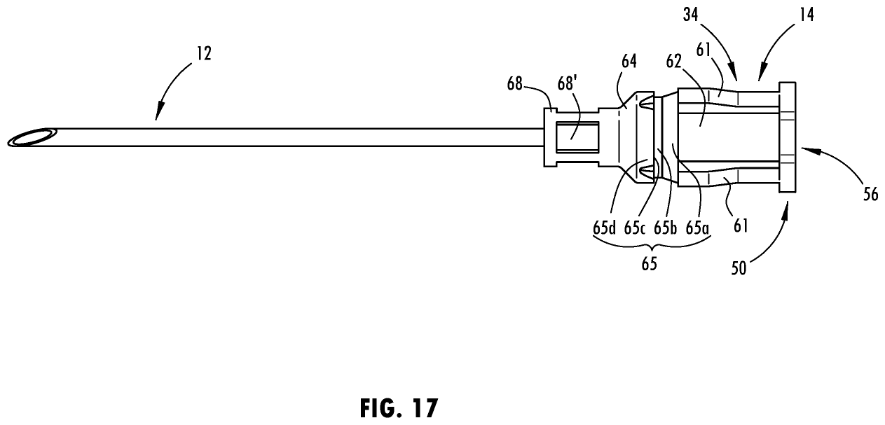

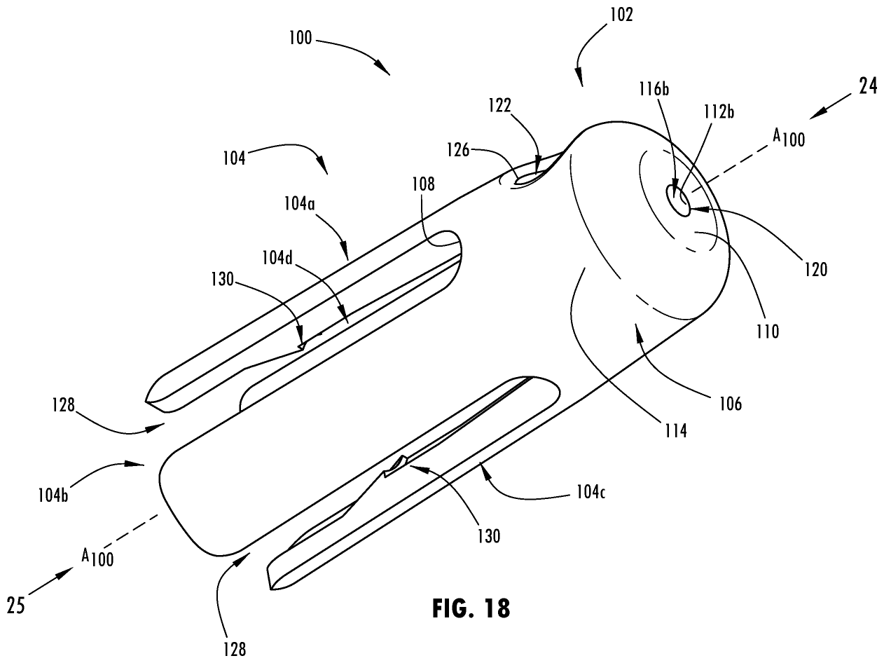

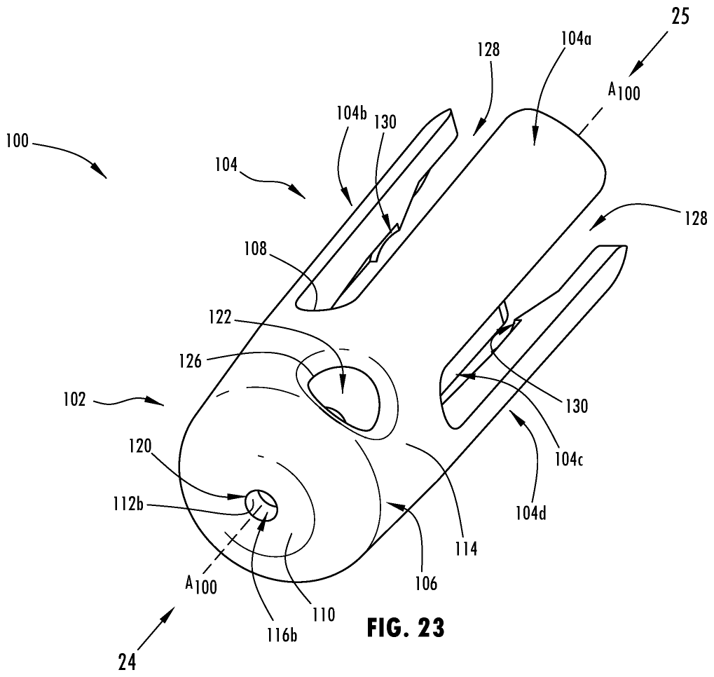

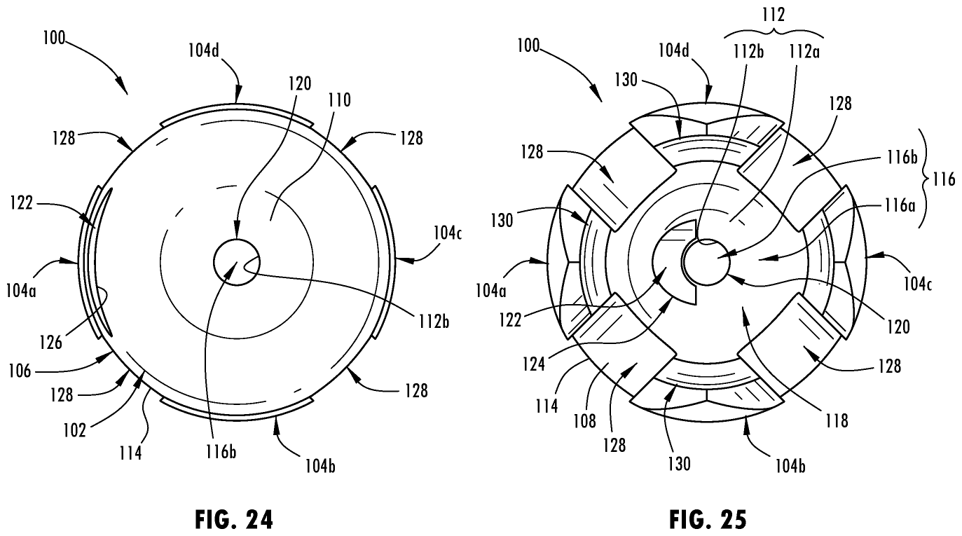

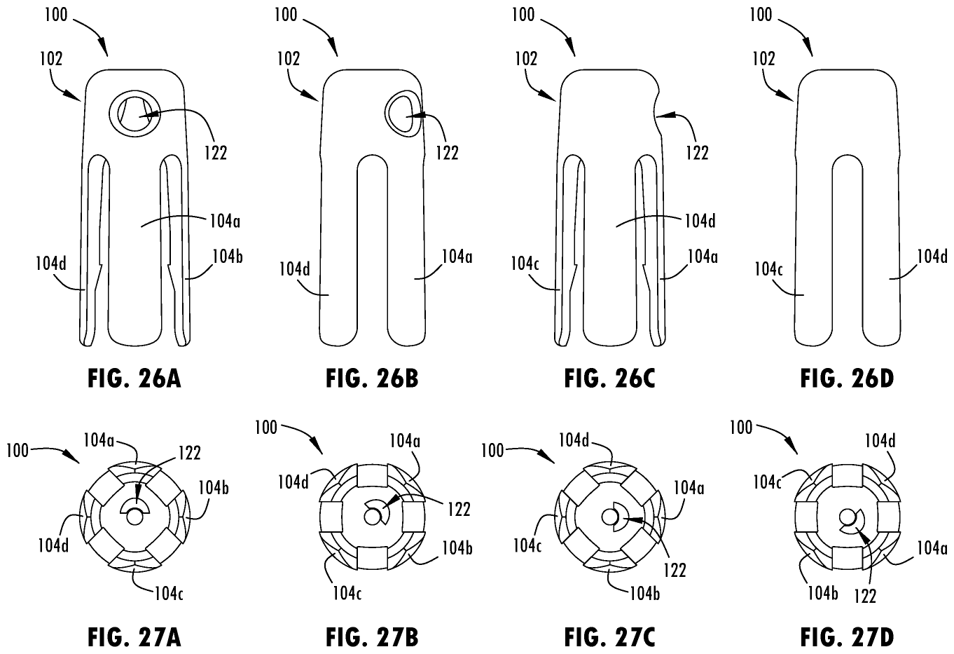

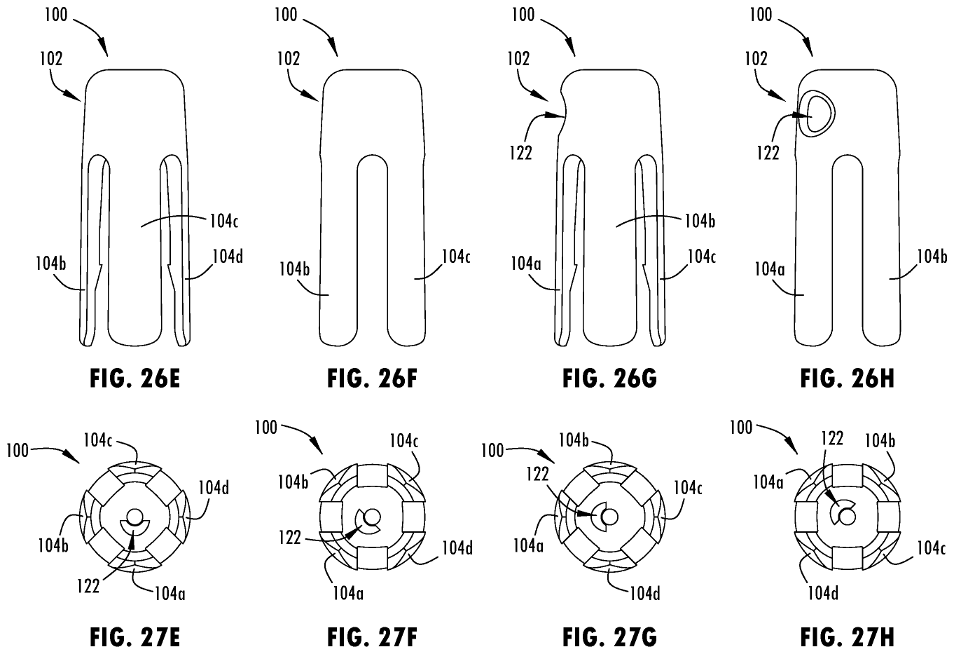

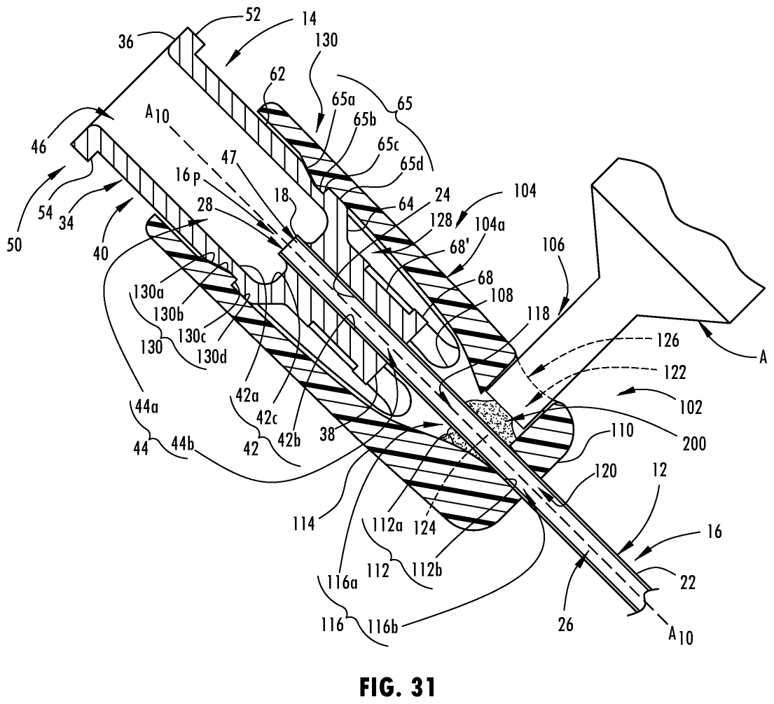

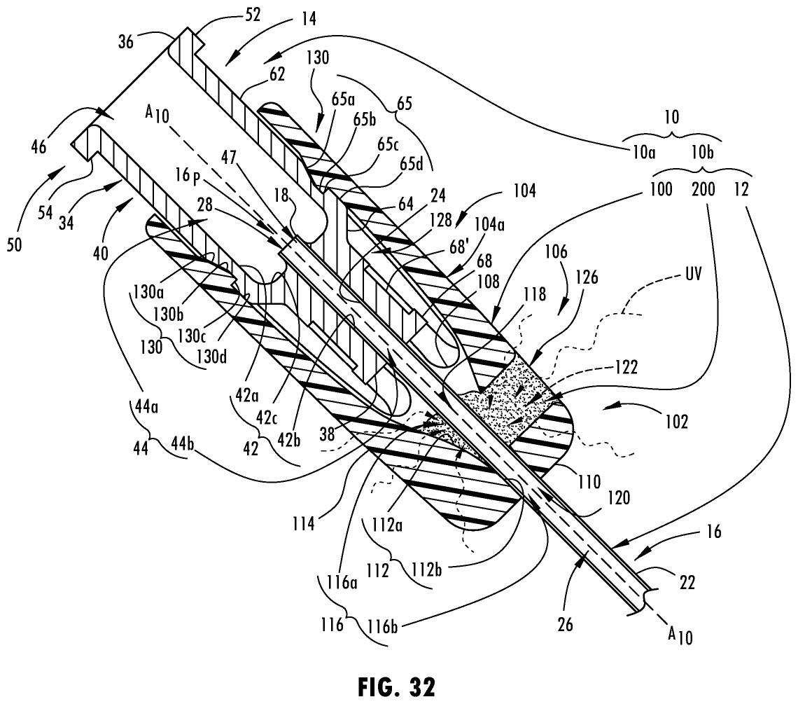

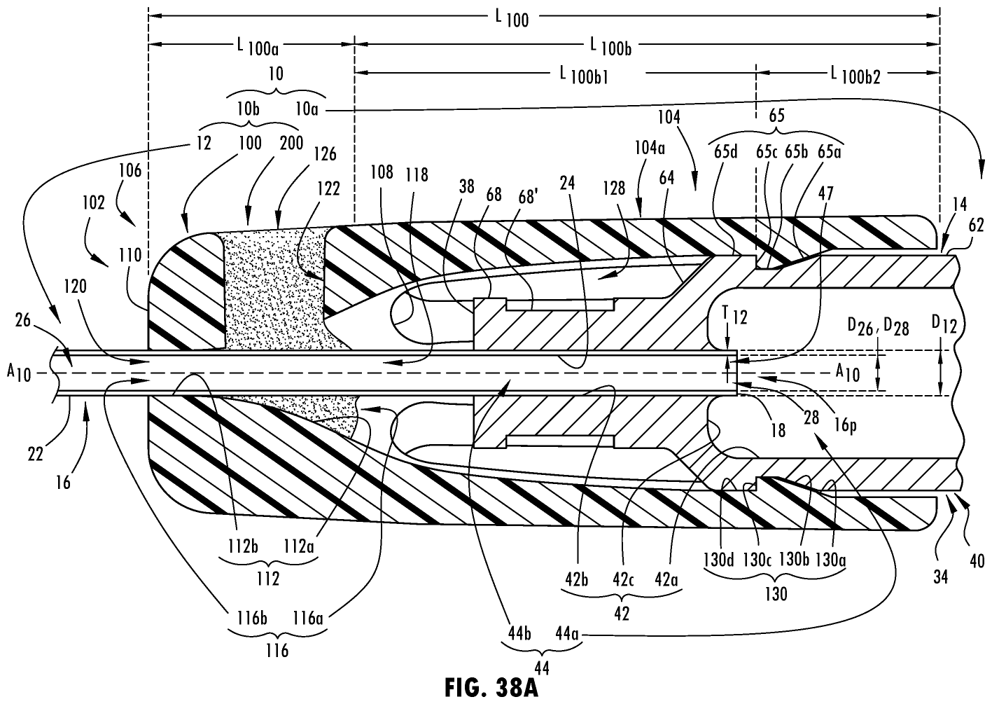

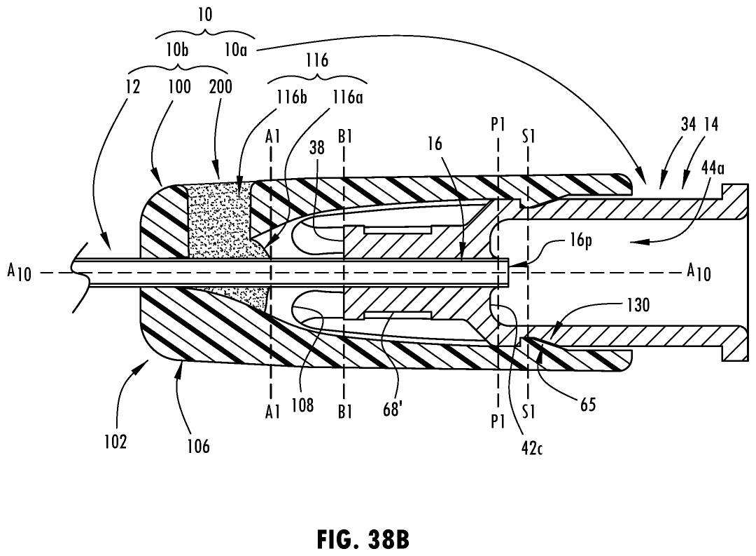

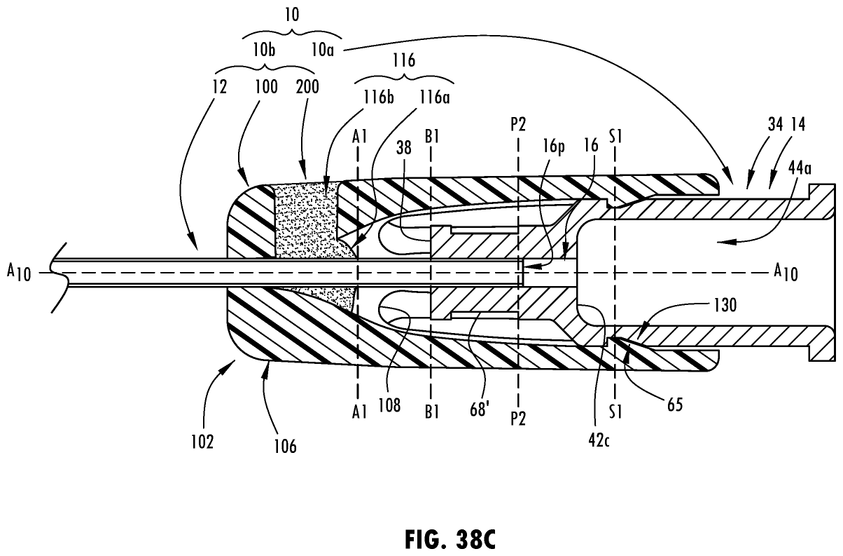

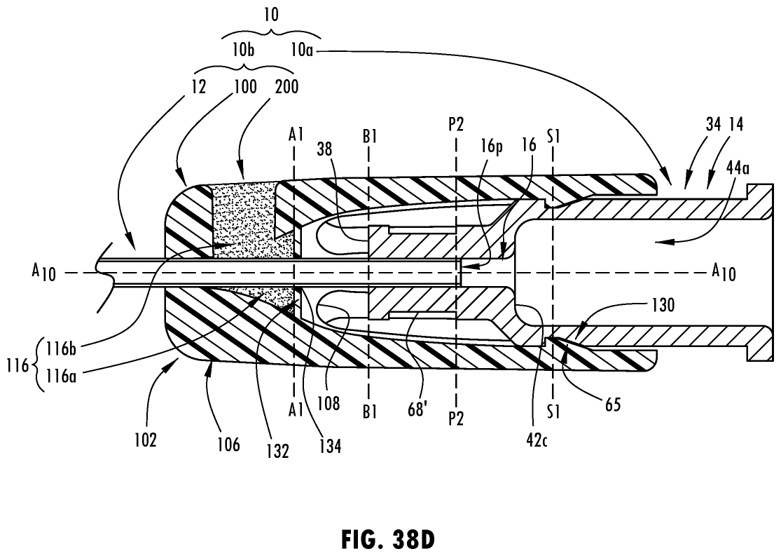

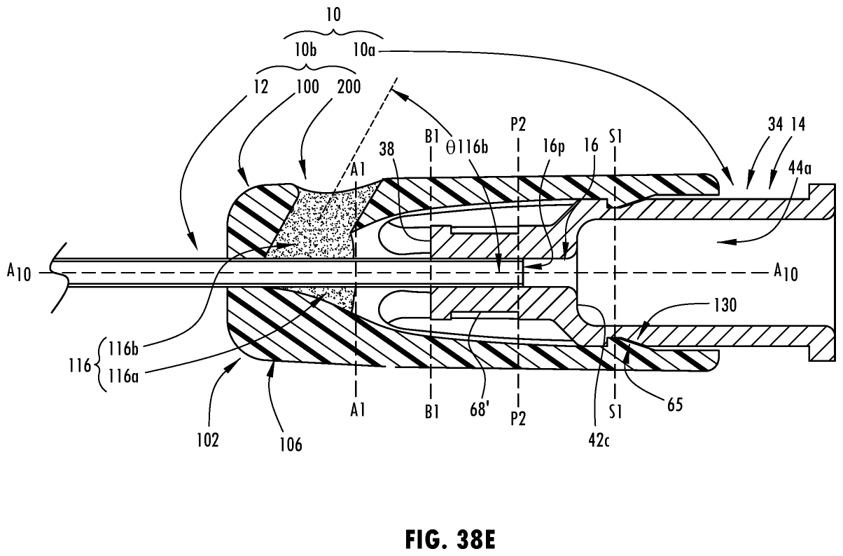

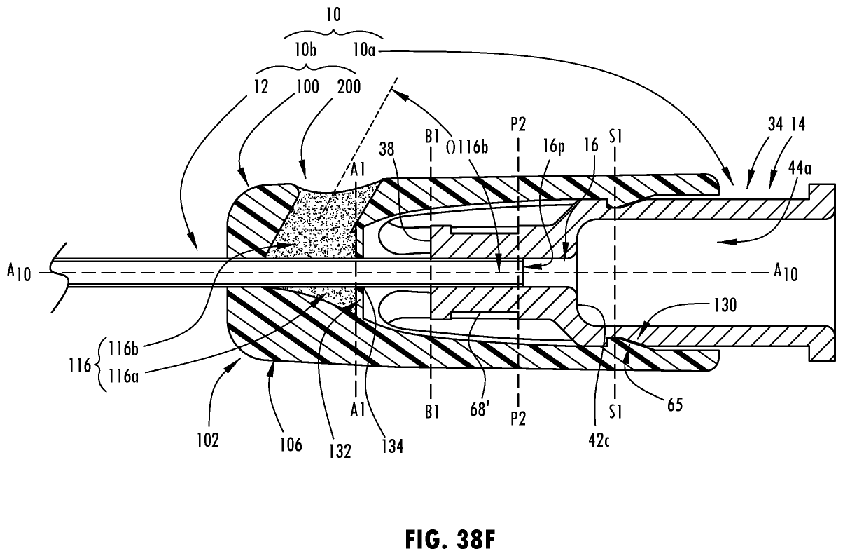

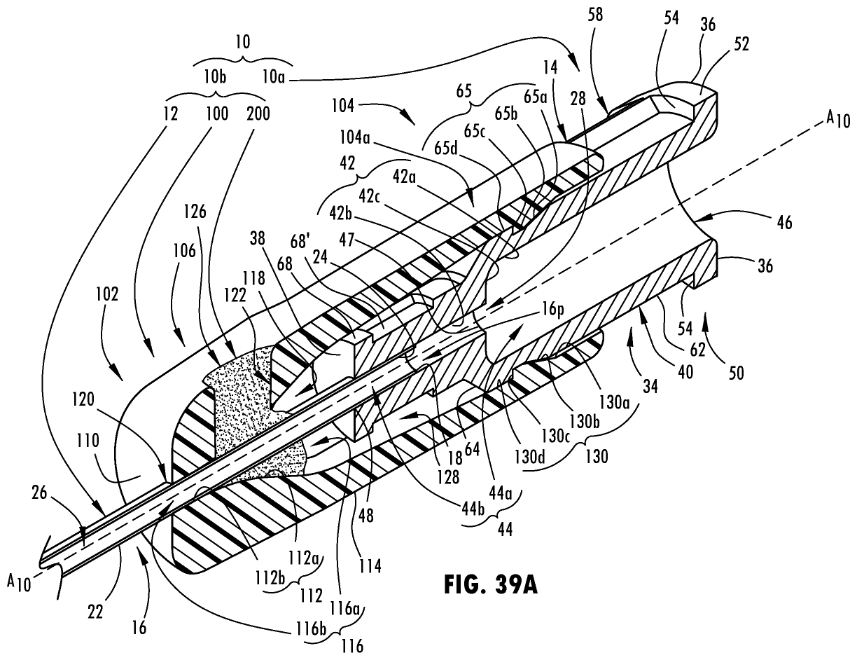

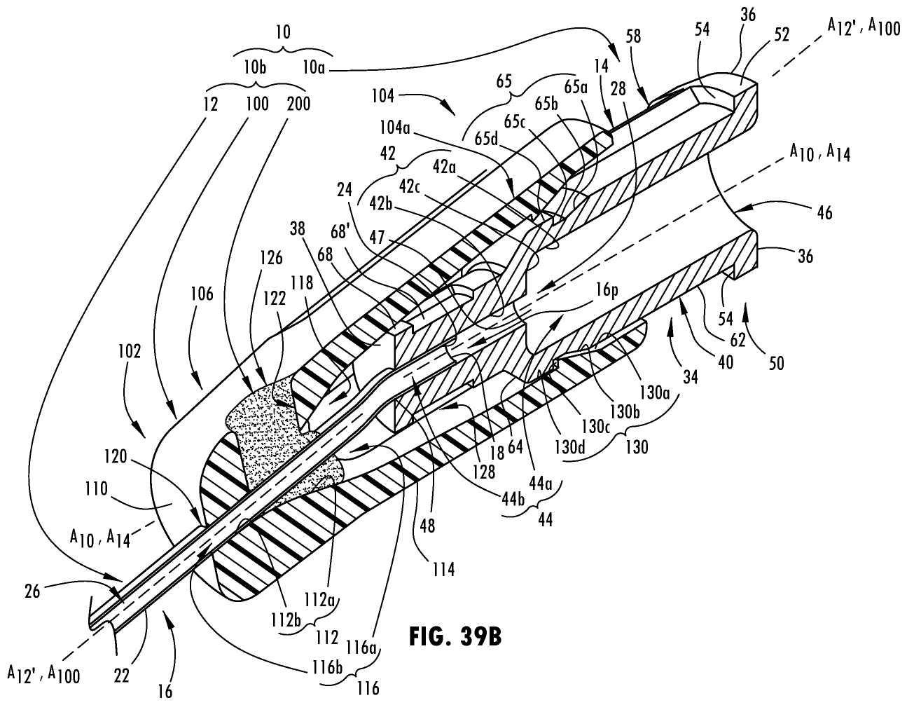

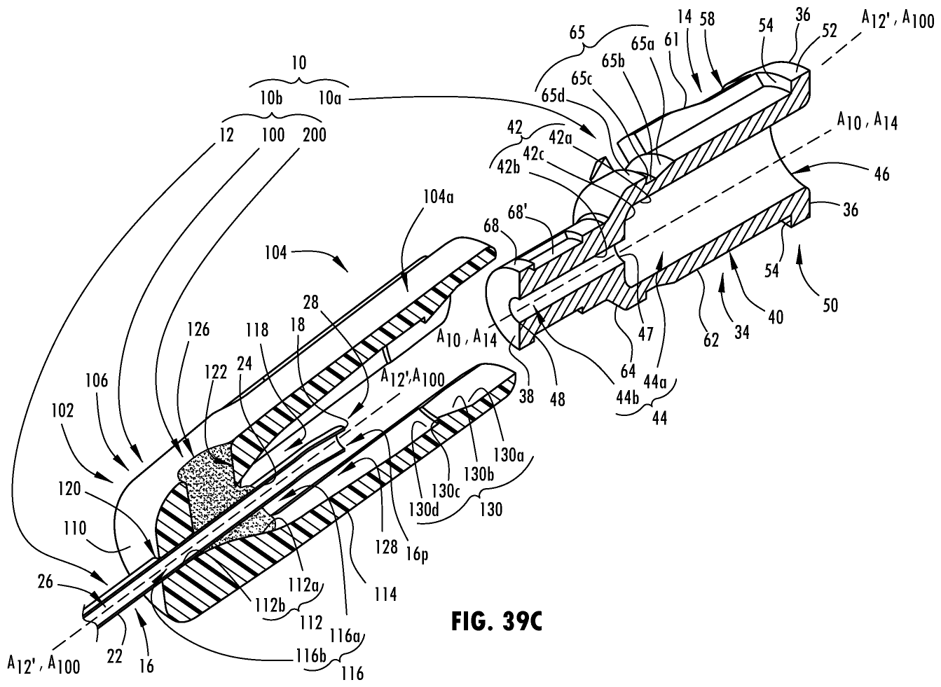

Referring to FIGS. 1 and 33-35B, a hypodermic interface assembly including a cannula 12 (see, e.g., FIGS. 1-2), a hub 14 (see, e.g., FIGS. 1 and 3-10), a cannula carrier 100 (see, e.g., FIGS. 18-27H), and an optional adhesive 200 (see, e.g., FIGS. 1 and 31-32) is shown generally at 10. Furthermore, a sub-assembly of the hypodermic interface assembly that is defined by the cannula 12 and the hub 14 is seen at FIGS. 11A-17. A central axis that extends through an axial center of each component (e.g., the cannula 12, the hub 14, and the cannula carrier 100) of the hypodermic interface assembly 10 is shown generally at A₁₀-A₁₀. As will be described in the following disclosure at FIGS. 31-32, the adhesive 200 is radially deposited through a radial passage (see, e.g., radial passage 122 of the cannula carrier 100) and the adhesive 200 surrounds a portion of the cannula 12 for optionally adhesively connecting the cannula 12 to at least the cannula carrier 100. Accordingly, the central axis A10-A₁₀ may also extend though an axial center of the adhesive 200 after it surrounds the cannula 12. An exemplary alternative configuration of the hypodermic interface assembly 10 is also seen at, for example, FIGS. 38C and 39A-39C and functions in a similar manner as the hypodermic interface assembly 10.

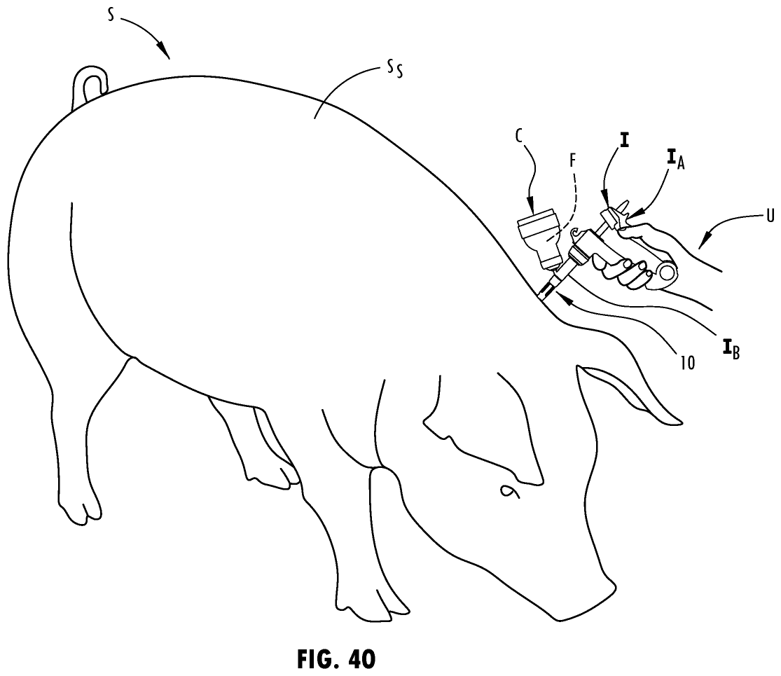

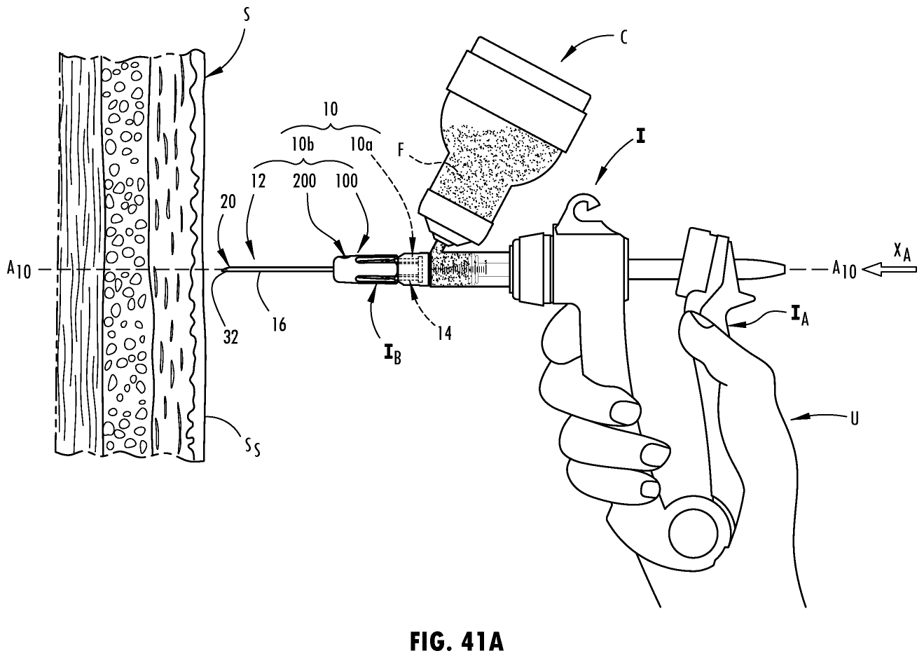

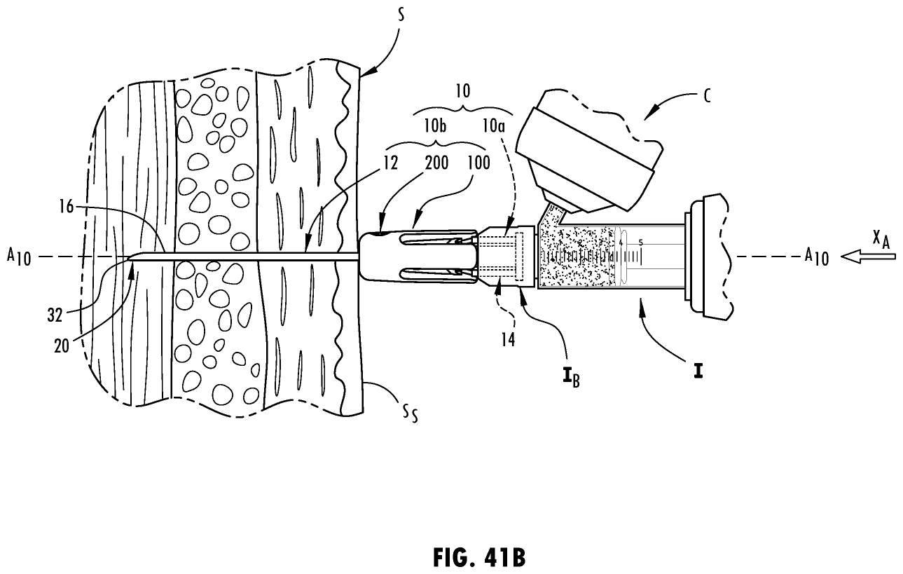

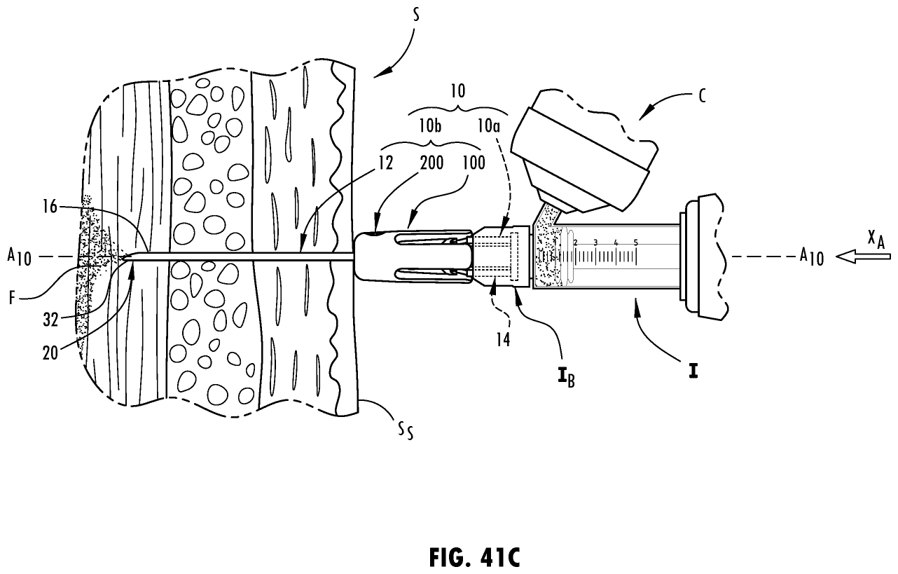

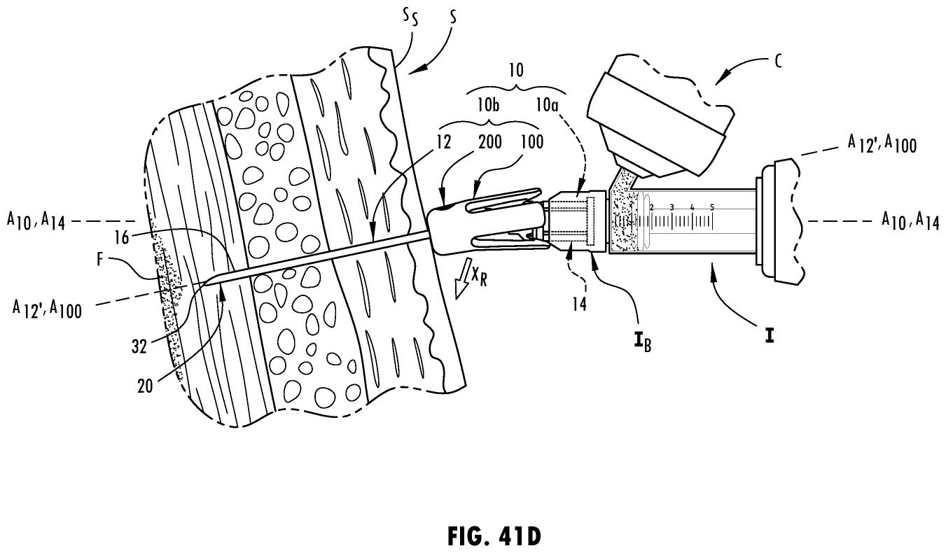

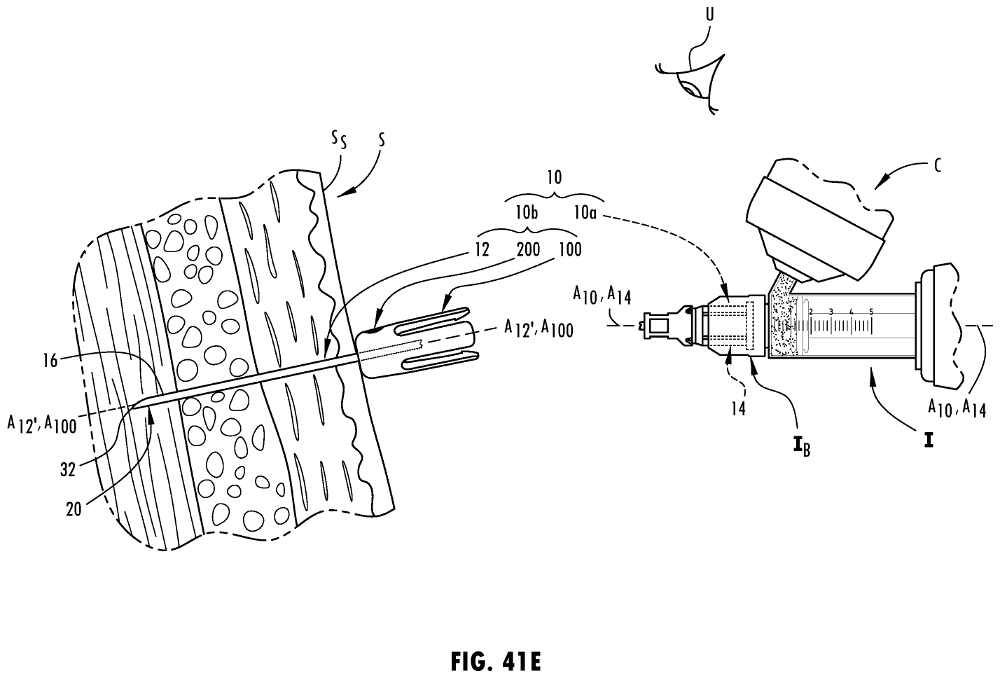

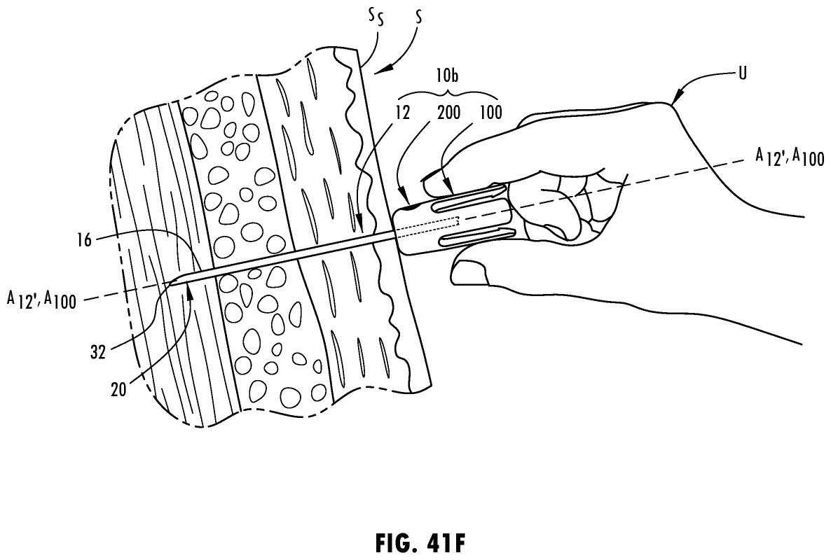

As seen at FIGS. 40 and 41A-41G, the cannula 12 is configured to pierce an outer surface S_{S} (e.g., the skin or hide) of a subject S (e.g., animalia, such as a human or non-human). The purpose of piercing the skin or hide S_{S} of the animalia S may be directed to injecting a fluid F (e.g., a medicament, a pharmaceutical, a vaccine, an anesthetic, or the like) into the animalia S as seen at, for example, FIG. 41C. In other examples, the purpose of piercing the skin or hide S_{S} of the animalia S may be directed to the purpose of drawing a fluid F (e.g., blood) from the animalia S. Accordingly, the cannula 12 may be referred to as a hypodermic cannula, and, as such, the assembly 10 may be referred to as a hypodermic interface assembly as a result of the cannula 12 being capable of injecting or drawing a fluid F into/from the animalia S.

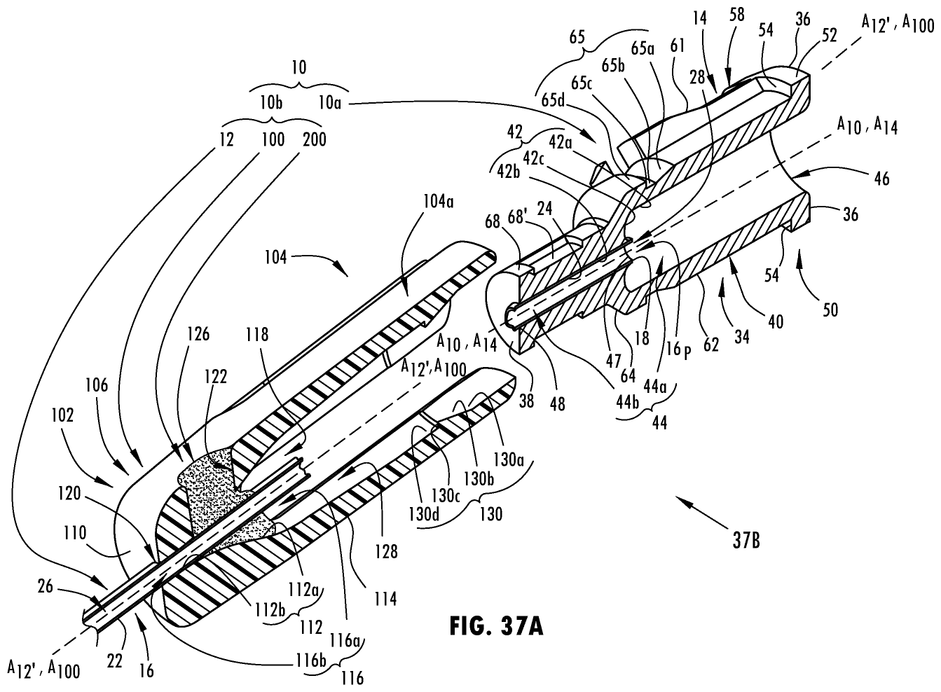

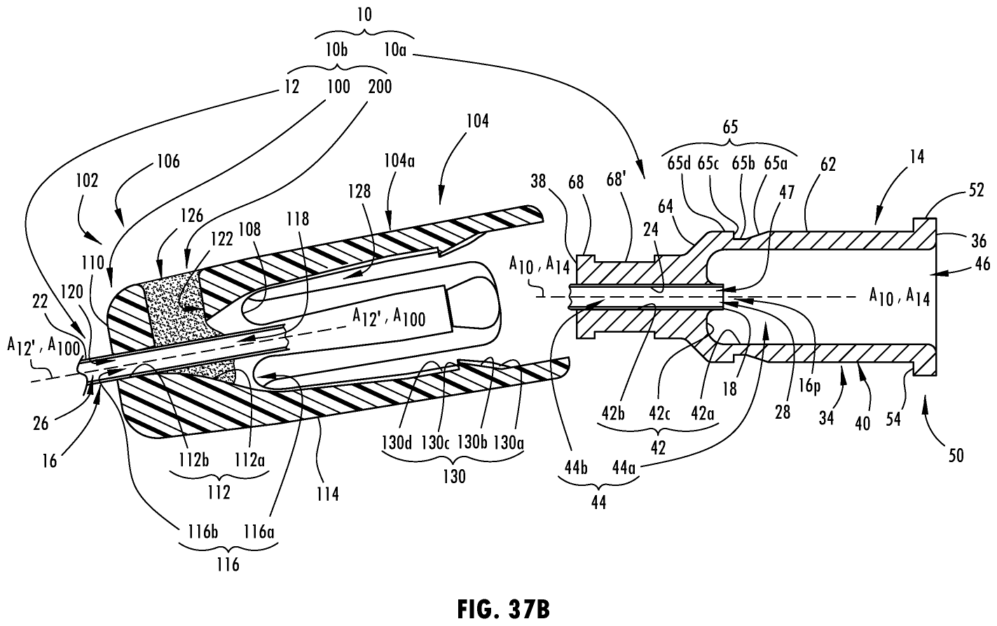

The design of the hypodermic interface assembly 10 provides for: (1) a first portion (see, e.g., a first portion 10a at FIGS. 37A-37B and 41E-41G) of the hypodermic interface assembly 10 that is configured to remain attached to an injection gun I after the cannula 12 is subjected to one or more radial forces X_{R} (see, e.g., FIG. 41D) relative to the central axis A₁₀-A₁₀ extending through the hypodermic interface assembly 10; and (2) a second portion (see, e.g., a second portion 10b at FIGS. 37A-37B and 41E-41G) of the hypodermic interface assembly 10 that is configured to controllably (and/or predictably) separate from the first portion 10a of the hypodermic interface assembly 10 after the cannula 12 is subjected to the one or more radial forces X_{R} relative to the central axis A₁₀-A₁₀ extending through the hypodermic interface assembly 10. With reference to FIG. 39C, in some configurations, the second portion 10b of the hypodermic interface assembly 10 includes the entirety of a length (see, e.g., L₁₂ in FIG. 2) of the cannula 12. In some instances, controlled separation of the second portion 10b of the hypodermic interface assembly 10 from the first portion 10a of the hypodermic interface assembly 10 may occur after the cannula 12 pierces the subject S (see, e.g., FIGS. 40 and 41B-41D). The subject S may be, for example, animalia, such as a human or non-human (i.e., an animal, such as a pig or swine). In other examples, the subject S may be an inanimate object. The predicable and controlled separation of the second portion 10b of the hypodermic interface assembly 10 from the first portion 10a of the hypodermic interface assembly 10 mitigates separation of the cannula 12 alone from a non-separated, non-broken, or unitary configuration of the hub 14, which may otherwise result in the cannula 12 being broken-off from the injection gun I and subsequently being lost within the flesh of the animalia.

As seen at FIG. 2, the cannula 12 is defined by a tube-shaped body 16 having a proximal end 16_{P} and a distal end 16D. The cannula 12 is defined by a length L₁₂ extending between the proximal end 16_{P} of the tube-shaped body 16 and the distal end 16D of the tube-shaped body 16. The length L₁₂ of the cannula 12 is defined by a plurality of sub-lengths L₁₂ₐ (including sub-length portions L₁₂ₐ₁, L₁₂ₐ₂, L₁₂ₐ₃, and L₁₂ₐ₄), L_{12b}, and L_{12c}, which will be further described in the following disclosure.

The cannula 12 may be formed using any desirable manufacturing procedure such as, for example: a drawing procedure, a molding procedure; a casting procedure; a machining procedure; a lathing procedure; or a combination thereof. The cannula 12 made from any desirable material such as, for example: a metallic material; a plastic material; or a combination thereof. In some examples, the cannula 12 may be made from a stainless steel material. In other instances, the cannula 12 may be made from an aluminum material. In yet other examples, the cannula 12 may be made from a detectable material such as, for example, a detectable alloy, a ferromagnetic alloy, a magnetically-detectable material, a magnetic resonance imaging (MRI) detectable material, a material that absorbs X-rays, or the like.

The cannula 12 may be defined in terms of ‘gauge size’ that takes into consideration skid/hide thickness of the subject S and/or a depth of injection of the subject S. The gauge size of the cannula 12 may be defined in a series of industry standard numbers in which, for example, the lower the number, the wider the diameter of the cannula. Furthermore, the series of industry standard numbers defining gauge size of the cannula 12 may be defined in a manner such that, for example, a higher gauge number indicates a smaller width of the cannula 12. In some instances, the industry standard gauge sizes of the cannula 12 may range from, for example: 14-Gauge; 16-Gauge; 18-Gauge; and 20-Gauge. Accordingly, in the range of exemplary industry standard numbers described above, a 14-Gauge cannula may be said to have a relatively largest diameter and highest strength (in terms of bendability/flexibility to a point where the cannula 12 could potentially break/fail) whereas a 20-Gauge cannula may be said to have a relatively smallest diameter and lowest strength (in terms of bendability/flexibility to a point where the cannula 12 could potentially break/fail).

A central axis A₁₂-A₁₂ extends through an axial center of the tube-shaped body 16 and along the length L₁₂ of the tube-shaped body 16. As will be described in the following disclosure and shown at FIG. 41D and at FIG. 2, a portion of the length L₁₂ of the tube-shaped body 16 (see, e.g., the sub-length portion L_{12c}) may bend, flex, or deviate from the central axis A₁₂-A₁₂ that extends through an axial center of the tube-shaped body 16. The sub-length portion L_{12c} of the length Lu of the tube-shaped body 16 that may bend, flex, or deviate from the central axis A₁₂-A₁₂ extends generally along an axis A₁₂′-A₁₂′ that may be said to be not aligned with and to deviate away from the central axis A₁₀-A₁₀ extending through the hypodermic interface assembly 10 when the cannula 12 is arranged as a component of the hypodermic interface assembly 10.

The tube-shaped body 16 is further defined by a proximal end surface 18 at the proximal end 16_{P} of the tube-shaped body 16 and a distal end surface 20 at distal end 16D of the tube-shaped body 16. The tube-shaped body 16 is further defined by an outer surface 22 extending between the proximal end surface 18 and the distal end surface 20. The tube-shaped body 16 is further defined by an inner surface 24 extending between the proximal end surface 18 and the distal end surface 20. The inner surface 24 further defines a passage 26 extending through the tube-shaped body 16. The proximal end surface 18 defines a proximal opening 28 that is in fluid communication with the passage 26. The distal end surface 20 defines a distal opening 30 that is in fluid communication with the passage 26.

With reference to FIG. 38A (which illustrates an enlarged cross-section view of an exemplary hypodermic interface assembly 10), the body 16 of the cannula 12 is defined by a thickness T₁₂ extending between the outer surface 22 of the body 16 and the inner surface 24 of the body 16. The outer surface 22 further defines an outer diameter D₁₂ of the cannula 12 that is referenced from the central axis A₁₂-A₁₂, which may be coincident with respective central axes A₁₀-A₁₀ and A₁₄-A₁₄ of each of the hypodermic interface assembly 10 and the hub 14. The inner surface 24 further defines the passage 26 to have a passage diameter D₂₆. The passage 26 is in fluid communication with the proximal opening 28 and the distal opening 30 in order to permit: (1) passage of a fluid F (see, e.g., FIG. 41C) into the tube-shaped body 16 at the proximal opening 28; (2) through the passage 26 in a direction from the proximal end 16_{P} of the tube-shaped body 16 and towards the distal end 16D of the tube-shaped body 16; and (3) out of the distal opening 30.

With reference to FIGS. 2 and 38A, the proximal end surface 18 extends from the outer surface 22 substantially perpendicularly, and, as such, defines the proximal end surface 18 to be blunted or non-sharpened. Furthermore, the proximal opening 28 formed by the proximal end surface 18 may define a substantially circular-shaped geometry that is defined by a proximal opening diameter D₂₈ that is substantially similar to the passage diameter D₂₆ of the passage 26.

With reference to FIG. 2, the distal end surface 20 extends from the outer surface 22 at a beveled angle θ₂₀, and, as such, the distal end surface 20 may be referred to as a beveled distal end surface that terminates at or defines a sharp piercing tip 32. The beveled distal end surface 20 may be defined by any desirable beveled angle θ₂₀ that forms, for example, a “standard bevel,” a “short bevel,” or a “true short bevel.” Because the beveled distal end surface 20 extends from the outer surface 22 at a beveled angle θ₂₀, the distal opening 30 may be defined by an oval-shaped geometry. In one embodiment, the distal end surface 20 may be defined by three separate beveled cuts.

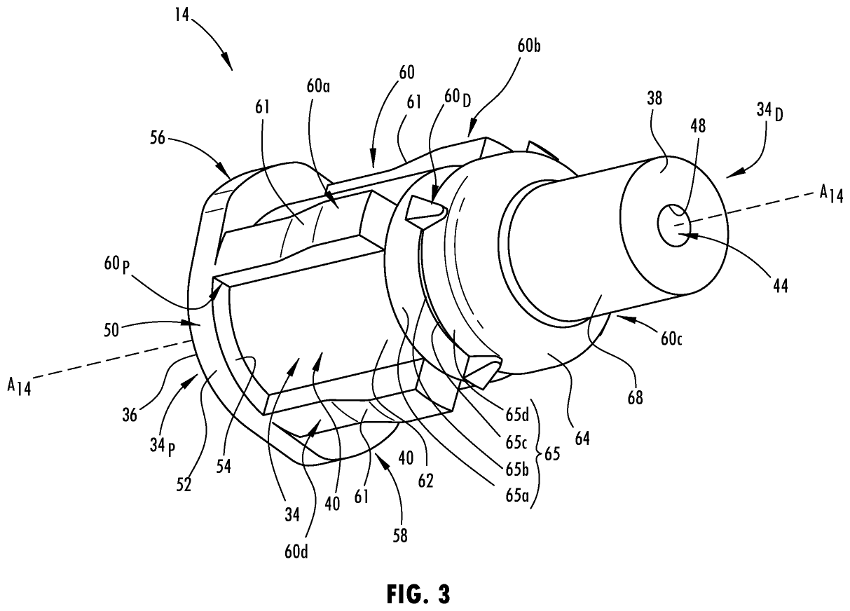

As seen at FIGS. 3-10, the hub 14 is defined by a substantially tube-shaped body 34 having a proximal end 34_{P} and a distal end 34_{D}. The hub 14 is defined by a length L₁₄ (see, e.g., FIG. 7) extending between the proximal end 34_{P} of the substantially tube-shaped body 34 and the distal end 34_{D} of the substantially tube-shaped body 34. The length L₁₄ of the hub 14 is defined by a plurality of sub-lengths L₁₄ₐ, L_{14b}, L_{14c}, and L_{14d}, which will be further described in the following disclosure.

The hub 14 may be formed using any desirable manufacturing procedure such as, for example: a molding procedure; a casting procedure; a machining procedure; a lathing procedure; or a combination thereof. The hub 14 made from any desirable material such as, for example: a metallic material; a plastic material; or a combination thereof. In some examples, the hub 14 may be made from a stainless steel material. In other instances, the hub 14 may be made from an aluminum material, brass, steel, or alloys. In other examples, the hub 14 may be made from plastic materials including but not limited to polypropylene (PP), polyethylene terephthalate (PET), polyamides (e.g., nylon 6, nylon 6, 6, thermosetting plastics such as polyester resins, epoxy resins, acrylics), and the like. Furthermore, in some instances, the hub 14 may be finished with an anodization, a polishing, an electro-polishing, a coating, a paint or the like with, for example, a highly visible finish (e.g., a dye, a fluorescent coating, a phosphorescent coating, a bright gloss, matt color finish, or the like that preferably is not similar to the flesh tone or color of the surface S_{S} of the flesh of the animalia).

The substantially tube-shaped body 34 is further defined by a proximal end surface 36 at the proximal end 34_{P} of the substantially tube-shaped body 34 and a distal end surface 38 at distal end 34_{D} of the substantially tube-shaped body 34. The substantially tube-shaped body 34 is further defined by an outer surface 40 extending between the proximal end surface 36 and the distal end surface 38. The substantially tube-shaped body 34 is further defined by an inner surface 42 extending between the proximal end surface 36 and the distal end surface 38.

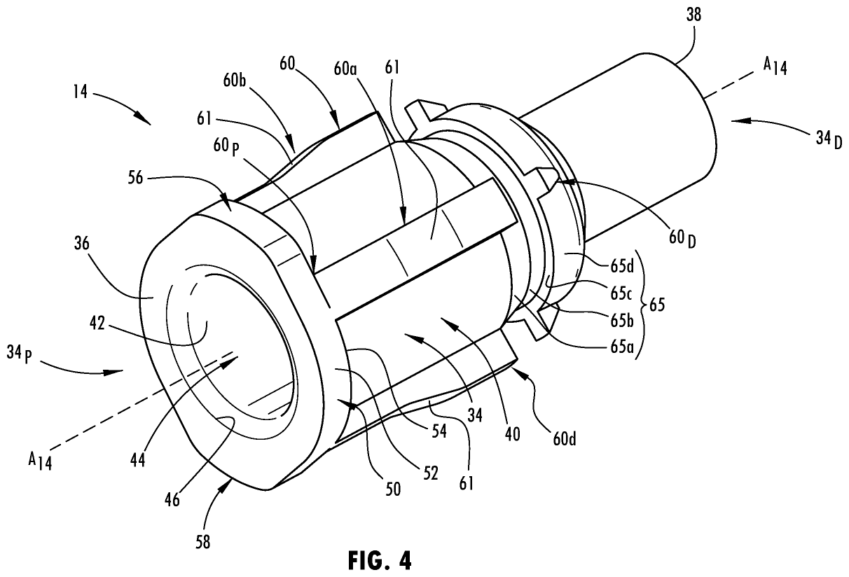

The inner surface 42 further defines a passage 44 extending through the substantially tube-shaped body 34. The proximal end surface 36 defines a proximal opening 46 (see, e.g., FIGS. 4, 7, and 10) that is in fluid communication with the passage 44. The distal end surface 38 defines a distal opening 48 (see, e.g., FIGS. 3, 5, 7, 8B, 9B) that is in fluid communication with the passage 44.

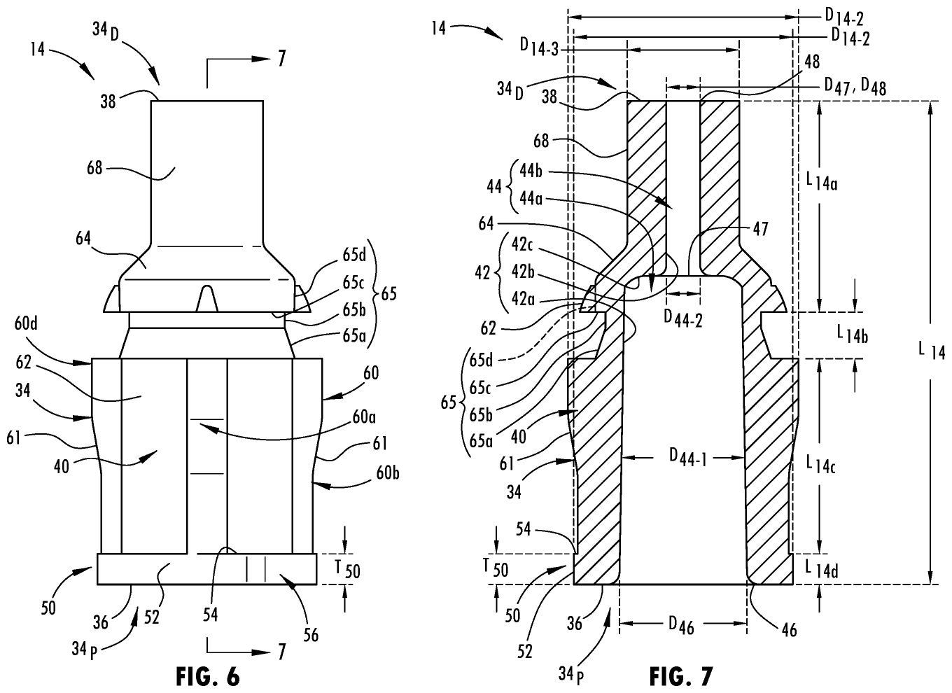

As seen at FIGS. 3-10, a ring portion 50 projects radially outwardly away from a central axis A₁₄-A₁₄ away from the outer surface 40 of the substantially tube-shaped body 34. The ring portion 50 may be alternatively referred to as a barrel-engaging portion that is configured to be connected to a barrel portion IB of an injection gun I (see, e.g., FIG. 40). The barrel-engaging portion 50 is defined by an outer side surface 52 that extends between the proximal end surface 36 and a distal shoulder surface 54. The barrel-engaging portion 50 may be defined by a thickness T₅₀ (see, e.g., FIGS. 6 and 7) extending between the proximal end surface 36 and the distal shoulder surface 54. The barrel-engaging portion 50 may generally define a Luer lock.

The outer surface 40 of the substantially tube-shaped body 34 may define a substantially circular-shaped geometry that defines a first outer diameter D₁₄₋₁ (see, e.g., FIG. 7) of the hub 14. The outer side surface 52 of the barrel-engaging portion 50 may define a substantially circular-shaped geometry that defines a second outer diameter D₁₄₋₂ (see, e.g., FIG. 7) of the hub 14. The second outer diameter D₁₄₋₂ of the hub 14 is greater than the first outer diameter D₁₄₋₁ of the hub 14. The outer surface 40 of the substantially tube-shaped body 34 may further define another substantially circular-shaped geometry that further defines a third outer diameter D₁₄₋₃ (see, e.g., FIG. 7) of the hub 14.

As seen at FIGS. 3-5, 8A-8B, 9A-9B, and 10, the substantially circular-shaped geometry of the outer side surface 52 of the barrel-engaging portion 50 is interrupted by a first radially-outward projection or ear 56 and a second radially-outward projection or ear 58 that extend beyond the second outer diameter D₁₄₋₂ of the hub 14. The first radially-outward projection or ear 56 may be arranged opposite of or offset approximately 180° from the second radially-outward projection or ear 58.

As seen at FIG. 7, the inner surface 42 of the substantially tube-shaped body 34 includes a first inner surface portion 42a, a second inner surface portion 42b, and a third inner surface portion 42c. Each of the first inner surface portion 42a and the second inner surface portion 42b generally circumscribe the central axis A₁₄-A₁₄ of the hub 14. The third inner surface portion 42c connects the first inner surface portion 42a to the second inner surface portion 42b; furthermore, the third inner surface portion 42c may be substantially orthogonal to the central axis A₁₄-A₁₄ of the hub 14. The third inner surface portion 42c may be substantially perpendicular with respect to each of the first inner surface portion 42a and the second inner surface portion 42b; in some implementations, the transition of each of the first inner surface portion 42a and the second inner surface portion 42b to the third inner surface portion 42c may be defines by a curved or arcuate segment. As will be seen in the following disclosure at FIGS. 11B-11C, after material deformation of at least a portion of, for example, the second portion 34b of the substantially tube-shaped body 34 of the hub 14 (e.g., by crimping a portion of, for example, the second portion 34b of the substantially tube-shaped body 34 of the hub 14 after the cannula 12 is interfaced with the hub 14 as seen as FIG. 11B), the curved or arcuate segment joining the second inner surface portion 42b to the third inner surface portion 42c may change in shape as a result of the material shifting or “flowing”, and, as such, a portion of the third inner surface portion 42c that extends from the second inner surface portion 42b may define a frustoconical surface portion (see, e.g., FIG. 11C) surrounding the cannula 12.

The first inner surface portion 42a of the inner surface defines a first passage portion 44a of the passage 44. The second inner surface portion 42b defines a second passage portion 44b of the passage 44.

The first passage portion 44a defines a first passage diameter D₄₄₋₁ (see, e.g., FIG. 7) of the passage 44. The second passage portion 44b defines a second passage diameter D₄₄₋₂ (see, e.g., FIG. 7) of the passage 44. The first passage diameter D₄₄₋₁ is greater than the second passage diameter D₄₄₋₂. The second passage diameter D₄₄₋₂ is approximately equal to but slightly greater than the outer diameter D₁₂ of the cannula 12.

The first passage portion 44a of the passage 44 is in fluid communication with the proximal opening 46, and the second passage portion 44b of the passage 44 is in fluid communication with the distal opening 48. Furthermore, the first passage portion 44a is in fluid communication with the second passage portion 44b by way of an intermediate opening 47. Accordingly, the passage 44 permits: (1) passage of a fluid F (see, e.g., FIG. 41C) into the substantially tube-shaped body 34 at the proximal opening 46; (2) through the first passage portion 44a of the passage 44 in a direction from the proximal end 34_{P} of the substantially tube-shaped body 34 and towards the intermediate opening 47 defined by the third inner surface portion 42c; (3) through the intermediate opening 47 that defines a proximal opening of the second passage portion 44b of the passage 44; (4) through the second passage portion 44b of the passage 44 in a direction from the intermediate opening 47 and towards the distal end 34_{D} of the substantially tube-shaped body34; and (5) out of the distal opening 48.

The proximal opening 46 formed by the proximal end surface 36 may define a substantially circular-shaped geometry that is defined by a proximal opening diameter D₄₆ (see, e.g., FIG. 7) that is substantially similar to the first passage diameter D₄₄₋₁ of the first passage portion 44a. The intermediate opening 47 formed by the third inner surface portion 42c of the inner surface 42 of the substantially tube-shaped body 34 may define a substantially circular-shaped geometry that is defined by an intermediate opening diameter D₄₇ (see, e.g., FIG. 7) that is substantially equal to the second passage diameter D₄₄₋₂. The distal opening 48 formed by the distal end surface 38 may define a substantially circular-shaped geometry that is defined by a distal opening diameter D₄₈ (see, e.g., FIG. 7) that is substantially similar to the second passage diameter D₄₄₋₂. Although some of the dimensions/diameters/geometries are descried above to be substantially similar or the same, the view of the hub 14 in the Figures (e.g., at FIG. 7) are exemplary and are not to scale. In some instances, the first passage portion 44a may be formed to include a draft angle (e.g., a 1° draft angle) that, for example, may assist in the removal of the hub 14 from tooling when the hub 14 is formed. Accordingly, the first passage diameter D₄₄₋₁ of the first passage portion 44a may progressively decrease in diameter as the first passage diameter D₄₄₋₁ of the first passage portion 44a extends in a direction from the proximal end surface 36 of the hub 14 toward the distal end surface 38 of the hub 14.

Referring to FIGS. 3-6, 8A, 8B, 9A, and 9B, one or more ribs 60 may project radially outwardly away from a central axis A₁₄-A₁₄ away from an outer body surface portion 62 defined by the outer surface 40 of the substantially tube-shaped body 34. The one or more ribs 60 may include, for example, a first rib 60a, a second rib 60b, a third rib 60c, and a fourth rib 60d.

The one or more ribs 60 may increase the structural integrity of the substantially tube-shaped body 34 of the hub 14. In some configurations, the one or more ribs 60 may arise from mold relief features during the manufacturing process of the substantially tube-shaped body 34 of the hub 14. Furthermore, the one or more ribs 60 may be configured to engage packaging (not shown). Engagement of the one or more ribs 60 with the packaging may assist in containing the cannula 12 and the hub 14 during shipping and/or assist in engagement/disengagement of the hub 14 with/from the injection gun I. As seen throughout the Figures, an outer surface portion of each rib 60a, 60b, 60c, 60d may extend radially outwardly, defining a lug portion; the lug portion may, for example be defined by an inclined or beveled surface 61. Each lug portion may be sized for engagement with the packaging.

Each rib 60a, 60b, 60c, 60d of the one or more ribs 60 includes a distal end 60D and a proximal end 60_{P}. The proximal end 60_{P} of each rib 60a, 60b, 60c, 60d of the one or more ribs 60 extends from the distal shoulder surface 54 of the barrel-engaging portion 50. The distal end 60_{D} of each rib 60a, 60b, 60c, 60d of the one or more ribs 60 extends in a direction toward the distal end surface 38 of the substantially tube-shaped body 34 and terminates at, before, or near an outer shoulder surface portion 64 (see, e.g., FIGS. 3-9) defined by the outer surface 40 of the substantially tube-shaped body 34. Each rib 60a, 60b, 60c, 60d of the one or more ribs 60 may define a substantially rectangular body that terminates with a substantially triangular body portion defined by the distal end 60D of each rib 60a, 60b, 60c, 60d of the one or more ribs 60.

The outer shoulder surface portion 64 extends from a distal-most end of the outer body surface portion 62 of the outer surface 40 of the substantially tube-shaped body 34. In some configurations, the outer shoulder surface portion 64 may define a dome-shaped or curved outer shoulder surface portion.

Referring to FIGS. 3-7, 8A, and 9A a distal-most end of the outer shoulder surface portion 64 terminates at an outer head surface portion 68. The outer head surface portion 68 generally circumscribes the central axis A₁₄-A₁₄ of the hub 14.

As seen at FIG. 7, the outer head surface portion 68 of the outer surface 40 of the substantially tube-shaped body 34 defines the third outer diameter D₁₄₋₃ of the hub 14. As seen at FIG. 7, the second outer diameter D₁₄₋₂ is greater than the third outer diameter D₁₄₋₃.

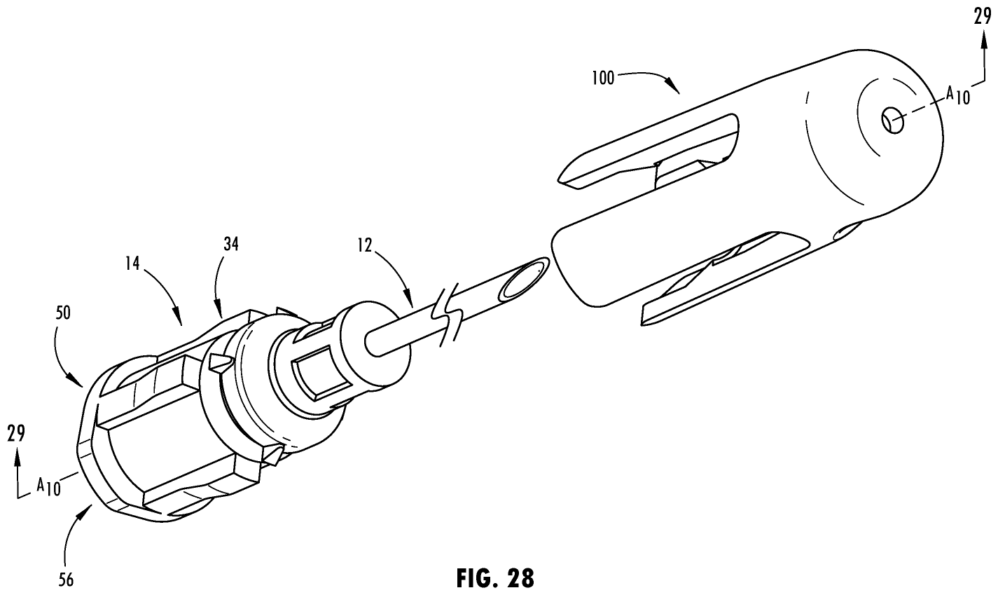

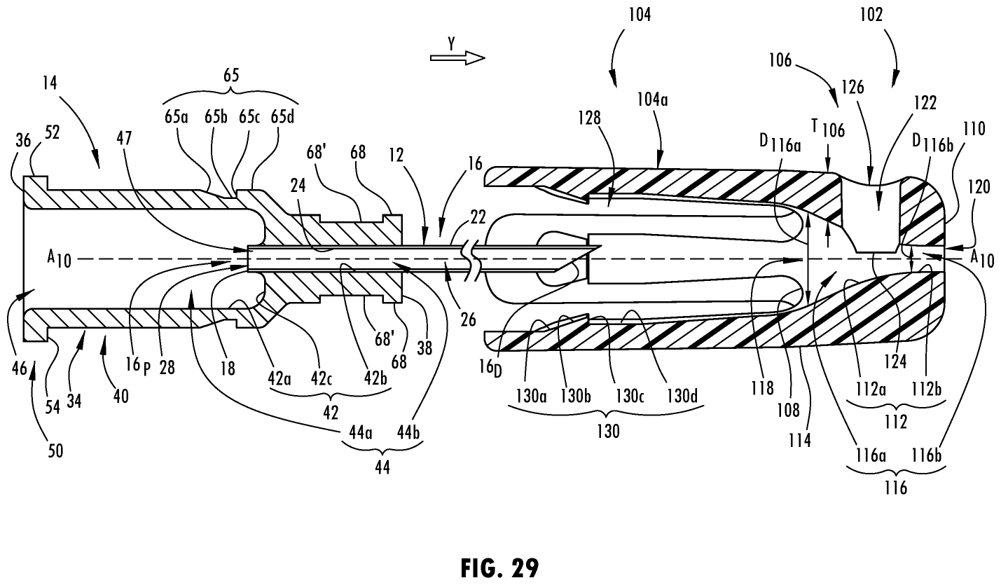

With reference to FIGS. 6-7, the hypodermic interface assembly 10 also includes a circumferential notch or groove 65 that is configured to receive one or more portions of the cannula carrier 100 (see, e.g., FIGS. 28-30). The circumferential notch or groove 65 may extend into the outer body surface portion 62 of the outer surface 40 of the substantially tube-shaped body 34 of the hub 14 at a distance (see, e.g., the sub-length L₁₄ₐ) away from the distal end surface 38 of the hub 14. In some configurations, the circumferential notch or groove 65 may extend into the outer surface 40 of the substantially tube-shaped body 34 near a proximal-most end of the outer shoulder surface portion 64. The circumferential notch or groove 65 may be defined by a plurality of surface portions (see, e.g., surface portions 65a, 65b, 65c, 65d at FIG. 29) of the outer surface 40 of the substantially tube-shaped body 34. The surface portions 65a, 65b, 65c, 65d that define the circumferential notch or groove 65 may be shaped to matingly-receive one or more corresponding surface portions (see, e.g., barb surface portions 130a, 130b, 130c, 130d) of the cannula carrier 100.

With reference to FIG. 7, the sub-length L₁₄ₐ defines the length of the outer shoulder surface portion 64 and the outer head surface portion 68 of the outer surface 40 of the substantially tube-shaped body 34. The sub-length L_{14b} defines the length circumferential notch or groove 65 that is defined by the outer body surface portion 62 of the outer surface 40 of the substantially tube-shaped body 34. The sub-length L_{14c} defines the length of the outer body surface portion 62 of the outer surface 40 of the substantially tube-shaped body 34 that extends between a proximal-most end of the circumferential notch or groove 65 and the distal shoulder surface 54 of the barrel-engaging portion 50. The sub-length L_{14d} defines a thickness of the barrel-engaging portion 50 that extends between the proximal end surface 36 of the substantially tube-shaped body 34 and the distal shoulder surface 54 of the barrel-engaging portion 50.

As will be described in the following disclosure, the substantially tube-shaped body 34 of the hub 14 may define first portion 10a of the hypodermic interface assembly 10 that is configured to remain attached to the injection gun I after the cannula 12 is subjected to one or more radial forces X_{R} relative to the central axis A₁₀-A₁₀ extending through the hypodermic interface assembly 10. The cannula carrier 100, which is removably-connected to the hub 14 at, for example, the circumferential notch or groove 65 defined by the outer body surface portion 62 of the outer surface 40 of the substantially tube-shaped body 34 of the hub 14, may define a first component portion of a second portion 10b of the hypodermic interface assembly 10 (with a second component portion of the second portion 10b of the hypodermic interface assembly 10 being the cannula 12 and a third component being the optional adhesive 200) that is configured to controllably separate from the first portion 10a of the hypodermic interface assembly 10 after the cannula 12 is subjected to the one or more radial forces X_{R} relative to the central axis A₁₀-A₁₀ extending through the hypodermic interface assembly 10. Accordingly, as will be explained in the following disclosure, upon predictably separating the cannula carrier 100 and the cannula 12 from the substantially tube-shaped body 34 of the hub 14, a user may easily locate and grasp (see, e.g., FIG. 41F) the cannula carrier 100 (that is non-removably-connected to the cannula 12) in order to remove the cannula 12 (see, e.g., FIG. 41G) from the flesh of the animalia S such that the cannula 12 is not lost within the flesh of the animalia S (should the one or more radial forces X_{R} relative to the central axis A₁₀-A₁₀ extending through the hypodermic interface assembly 10 be imparted to the cannula 12 during the course of utilizing the hypodermic interface assembly 10, which may otherwise undesirably result in the cannula 12 being separated from the injection gun I).

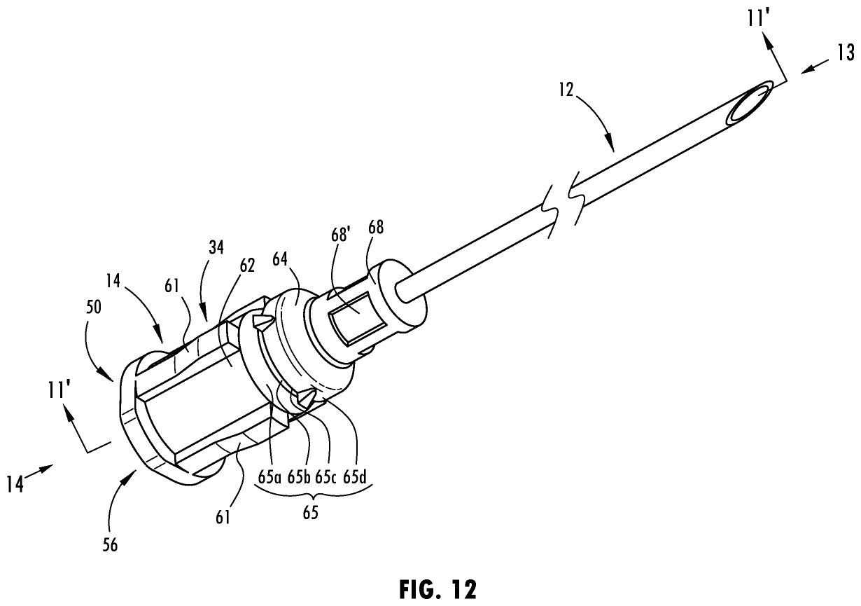

Referring to FIGS. 11A-11C, a method for assembling a sub-assembly (defined by the cannula 12 and the hub 14) of the hypodermic interface assembly 10 (the sub-assembly of which is shown in assembled form at FIGS. 12-17, 28 and 29) is described. Firstly, at FIG. 11A, the components (i.e., the cannula 12 and the hub 14 of the sub-assembly are axially aligned about a central axis A₁₀-A₁₀ (see also FIG. 1). The central axis A₁₀-A₁₀ corresponds to, for example, the central axes A₁₂-A₁₂, A₁₄-A₁₄ of each of the cannula 12 and the hub 14.

As will be described in the following disclosure, the cannula 12 is mechanically joined to any portion of the hub 14 as a result of, for example, material deformation of at least a portion of, for example, the outer head surface portion 68 of the outer surface 40 of the substantially tube-shaped body 34 of the hub 14 (e.g., by crimping a portion of, for example, outer head surface portion 68 of the outer surface 40 of the substantially tube-shaped body 34 of the hub 14 as seen as FIGS. 11B and 11C). Although the sub-assembly of the hypodermic interface assembly 10 is formed by a mechanical connection, the cannula 12 may alternatively or additionally be joined to any portion of the hub 14, such as, for example, with an adhesive (not shown), such as, for example: an acrylic adhesive, a cyanoacrylate adhesive, a ultra-violet (UV) curable adhesive, or the like. In other configurations the hub 14 may be attached to the cannula 12 by over-molding a material defining the hub 14 relative the cannula 12 (e.g., when the hub 14 is formed from a moldable material such as a plastic material).

As seen at FIG. 11A, a portion of the cannula 12 including the proximal end surface 18 at the proximal end 16_{P} of the tube-shaped body 16 is shown arranged near the distal opening 48 (that is in fluid communication with the second passage portion 44b of the passage 44 of the hub 14) formed by the distal end surface 38 of the hub 14. The central axis A₁₂-A₁₂ (see, e.g., FIG. 2) of the cannula 12 is axially aligned with the central axis A₁₄-A₁₄ of the hub 14. The central axes A₁₂-A₁₂ and A₁₄-A₁₄ of each of the cannula 12 and the hub 14 correspond to the central axis A₁₀-A₁₀ (see FIG. 1) of the hypodermic interface assembly 10.

As described above, the outer surface 22 of the tube-shaped body 16 of the cannula 12 defines an outer diameter D₁₂ of the cannula 12, and the second passage diameter D₄₄₋₂ (that defines the second passage portion 44b of the passage 44) is approximately equal to but slightly greater than the outer diameter D₁₂ of the cannula 12 so that at least a portion of the second passage portion 44b of the passage 44 is configured to receive the cannula 12. Then, as seen at FIGS. 11B-11C, the proximal end 16_{P} of the tube-shaped body 16 of the cannula 12 is inserted (according to the direction of the arrow Y as seen at FIG. 11A) through the distal opening 48 formed by the distal end surface 38 of the hub 14 and then disposed within at least a portion of the second passage portion 44b of the passage 44 of the hub 14. In some configurations as seen at, for example, FIGS. 11C and 38A-38B, the cannula 12 may be arranged relative the hub 14 such that the proximal end 16_{P} of the tube-shaped body 16 of the cannula 12 is located beyond the third inner surface portion 42c (see, e.g., dashed line P1 at FIG. 38B) of the inner surface 42 of the substantially tube-shaped body 34. As such, a portion of the cannula 12 is arranged within and entirely occupies the second passage portion 44b of the passage 44 of the hub 14 while also being partially disposed within the first passage portion 44a of the passage 44 of the hub 14.

Thereafter, as seen at FIG. 11B, the cannula 12 is arranged within the passage 44 of the hub 14 in order to subsequently, for example, mechanically join the cannula 12 to the hub 14 by, for example, arranging the head surface portion 68 of the outer surface 40 of the substantially tube-shaped body 34 of the hub 14 within, for example, a crimping tool T. The crimping tool T may punch, crimp, swage or materially deform, for example, all or a portion of the head surface portion 68 of the outer surface 40 of the substantially tube-shaped body 34 of the hub 14 in order to mechanically connect all or a portion of the second inner surface portion 42b of the inner surface 42 of the substantially tube-shaped body 34 of the hub 14 to a portion of the length (see, e.g., sub-length L₁₂ₐ₂ at FIG. 2) of the outer surface 22 of the tube-shaped body 16 of the cannula 12 in a friction-fit relationship, an interference-fit relationship, or a mechanically-coupled relationship.

Accordingly, as seen at FIG. 11C, the cannula 12 may be mechanically joined to the hub 14 as a result of the material deformation of the portion of the hub 14 by the crimping tool T. In some configurations as seen at FIG. 11C and FIGS. 12 and 15-17 and 28, the crimping tool T may punch, crimp, swage or materially deform, for example, a portion of the outer surface 40 of the substantially tube-shaped body 34. In some implementations, for example, the crumping tool T may punch, crimp, swage or materially deform (see, e.g., reference numeral 68′), for example, a portion or all of the outer head surface portion 68 of the outer surface 40 of the substantially tube-shaped body 34. Accordingly, in such implementations, the outer head surface portion 68 of the outer surface 40 of the substantially tube-shaped body 34 may define crimping pockets (see, e.g., reference numeral 68′) that infer material deformation of the outer head surface portion 68 of the outer surface 40 of the substantially tube-shaped body 34, and, as a result, distinguishes a “deformed” hub 14 that is mechanically connected to the cannula 12 from a virgin or “non-deformed” hub 14 (see, e.g., FIGS. 3-10) having an outer head surface portion 68 of the outer surface 40 of the substantially tube-shaped body 34 that does not define crimping pockets 68′.

As described above, a portion of the length (e.g., sub-length L₁₂ₐ₂) of the outer surface 22 of the tube-shaped body 16 of the cannula 12 may be disposed within the second passage portion 44b of the passage 44 of the hub 14 and may be mechanically secured (see, e.g., FIG. 11C) to at least a portion of the second inner surface portion 42b of the inner surface 42 of the substantially tube-shaped body 34 of the hub 14, which may extend along a sub-length of the hub 14 defined by the length L₁₄ₐ (see, e.g., FIG. 7) of the outer head surface portion 68 of the outer surface 40 of the substantially tube-shaped body 34 of the hub 14. Additionally, with reference to FIGS. 11C and 38A, a further portion of the length (e.g., sub-length L₁₂ₐ₃ at FIG. 2) of the outer surface 22 of the tube-shaped body 16 of the cannula 12 may be disposed within the second passage portion 44b of the passage 44 of the hub 14, and may extend along the sub-length of the hub 14 defined by the length L₁₄ₐ the head surface portion 68 of the outer surface 40 of the substantially tube-shaped body 34 of the hub 14. As such, in some implementations, a portion of the sub-length L₁₄ₐ of the length L₁₄ of the hub 14 may not be materially deformed by the crimping tool T. For example, the sub-length L₁₂ₐ₃ of the cannula 12 may not be mechanically coupled to the hub 14 along the portion of the sub-length L₁₄ₐ of the head surface portion 68 of the outer surface 40 of the substantially tube-shaped body 34 of the hub 14.

Furthermore, as seen at FIGS. 11C and 38A, a portion of the length (see, e.g., length portion L₁₂ₐ₄) of the outer surface 22 of the tube-shaped body 16 of the cannula 12 may be disposed within the second passage portion 44b of the passage 44 of the hub 14, which may extend along a sub-length of the hub 14 defined by a portion of the length L₁₄ₐ of the outer shoulder surface portion 64 of the outer surface 40 of the substantially tube-shaped body 34 of the hub 14. With reference to FIGS. 2,11C, and 38A a remainder/length portion (see, e.g., length portion L_{12c}) of the outer surface 22 of the tube-shaped body 16 of the cannula 12 extends beyond the distal end surface 38 of the hub 14 and is not contained within the passage 44 of the hub 14.

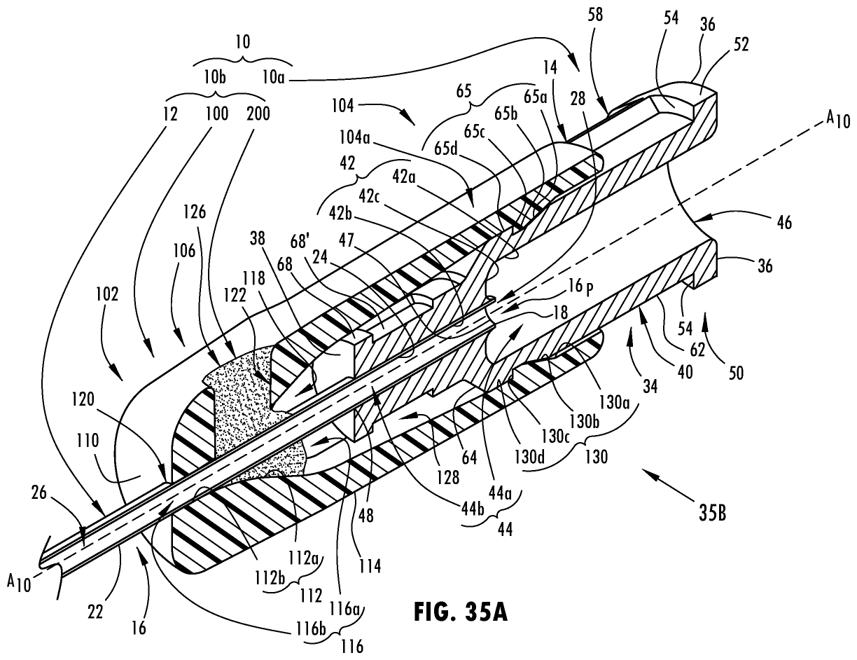

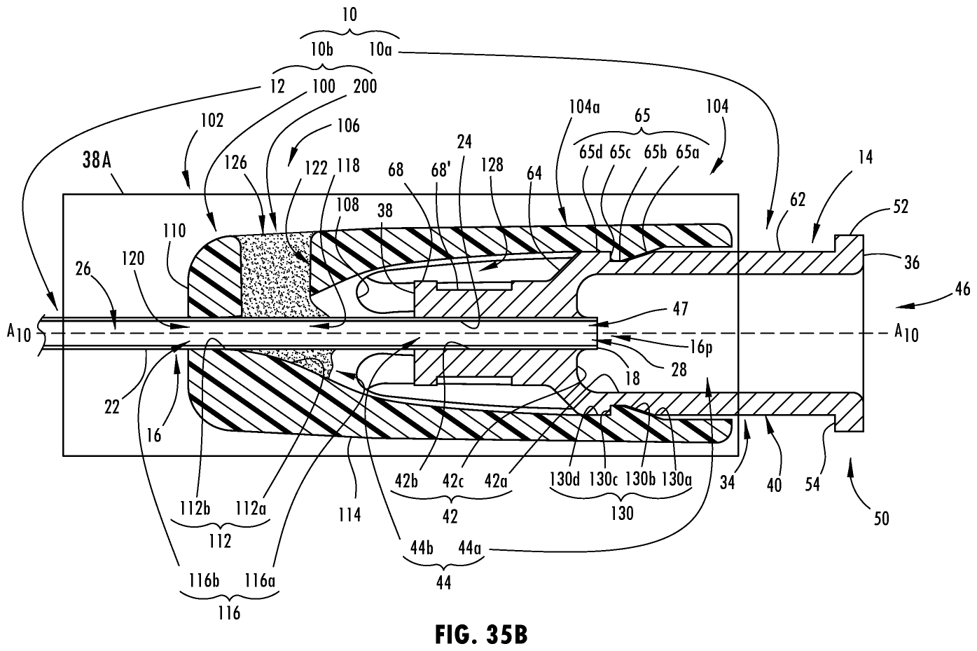

With reference to FIGS. 28-32, in addition to the cannula 12 being joined to the hub 14 to define the sub-assembly (defined by the cannula 12 and the hub 14), the subassembly may be joined to the cannula carrier 100 to further define the hypodermic interface assembly 10 (and the adhesive 200 may be deposited and then cured). The hypodermic interface assembly 10 may be joined to an injection gun I (see, e.g., FIG. 40). As will be described in the following disclosure at FIGS. 41A-41G, the second portion 10b of the hypodermic interface assembly 10 is configured to controllably separate from the first portion 10a of the hypodermic interface assembly 10 (see, e.g., FIGS. 35A-35B, 36A-36B, and 37A-37B). With reference to FIGS. 18-27H, an exemplary cannula carrier 100 is now described.

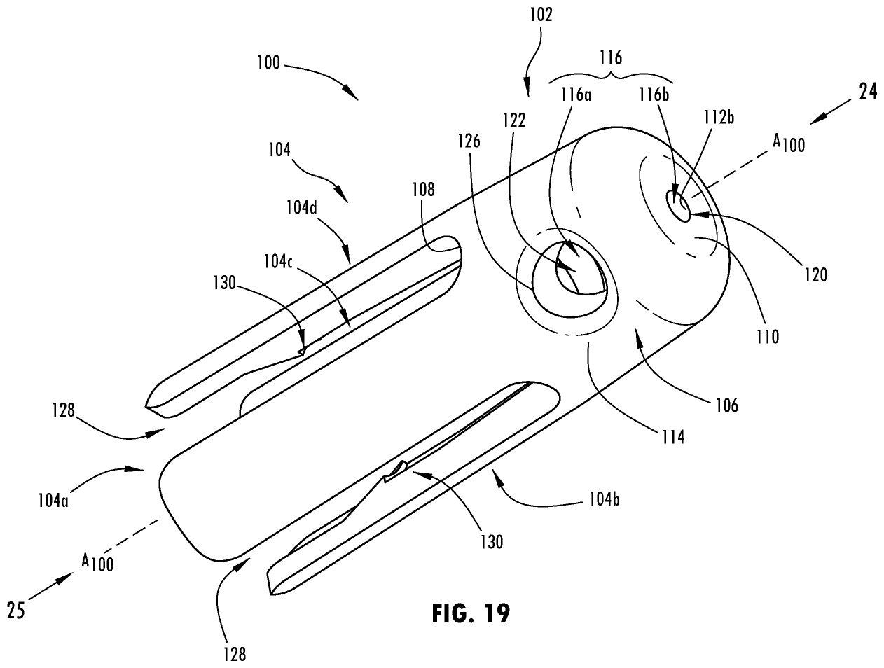

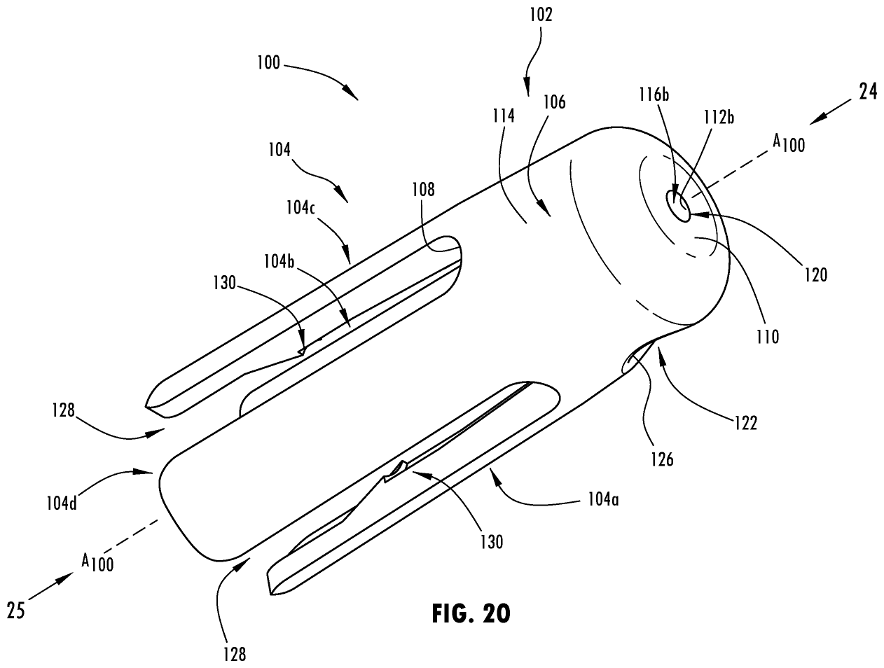

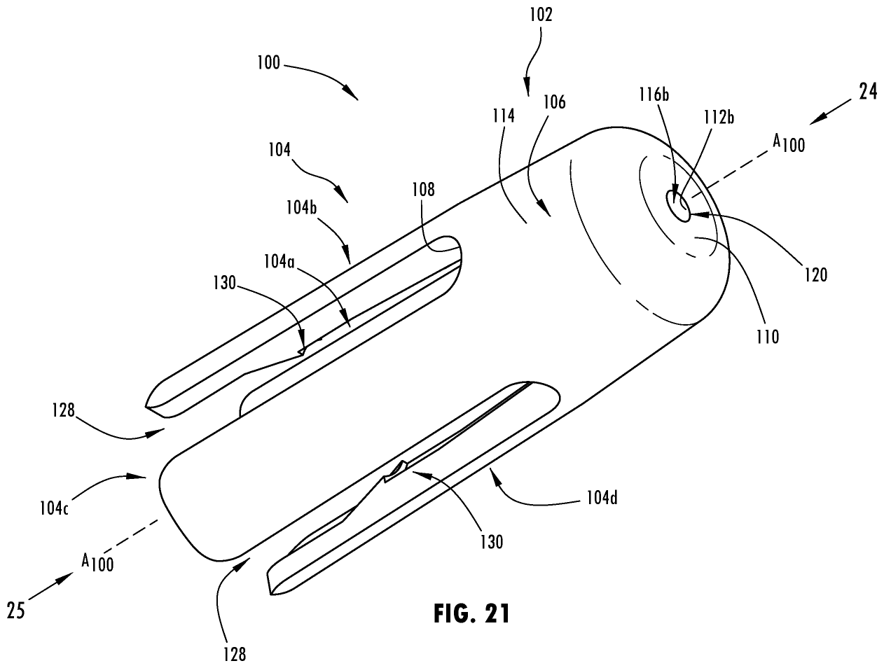

Referring to FIGS. 18-21, an exemplary cannula carrier 100 includes a head portion 102 and a plurality of leg portions 104 defined by, for example, four leg portions including a first leg portion 104a (see, e.g., FIG. 19), a second leg portion 104b (see, e.g., FIG. 18), a third leg portion 104c (see, e.g., FIG. 21), and a fourth leg portion 104d (see, e.g., FIG. 20). The head portion 102 includes a body 106 extending between a proximal end surface 108 and a distal end surface 110. The body 106 is also defined by a thickness T₁₀₆ (see, e.g., FIG. 29) extending between an inner surface 112 (see, e.g., FIG. 29) and an outer surface 114. The cannula carrier 100 may be formed using any desirable manufacturing procedure such as, for example: a molding procedure; a casting procedure; a machining procedure; or a combination thereof. The cannula carrier 100 may be made from any desirable material such as, for example: a metallic material; a plastic material; or a combination thereof. In some examples, the cannula carrier 100 may be made with a high visibility dye or pigment such as, for example, a brightly colored pigment, a fluorescent pigment, a phosphorescent pigment, retroreflective partially mirrored glass beads, metallic flake pigment, or the like that preferably is not similar to the flesh tone or color of the surface S_{S} of the flesh of the animalia. Furthermore, when the cannula carrier 100 is formed, the cannula carrier 100 may include an over-molded RFID component (not shown) embedded in the material or an RFID sticker (not shown) or other identifying information disposed upon one or more of the inner surface 112 and the outer surface 114 of the cannula carrier 100 in order to determine the location and/or serial number or other identifier associated with the cannula carrier 100.

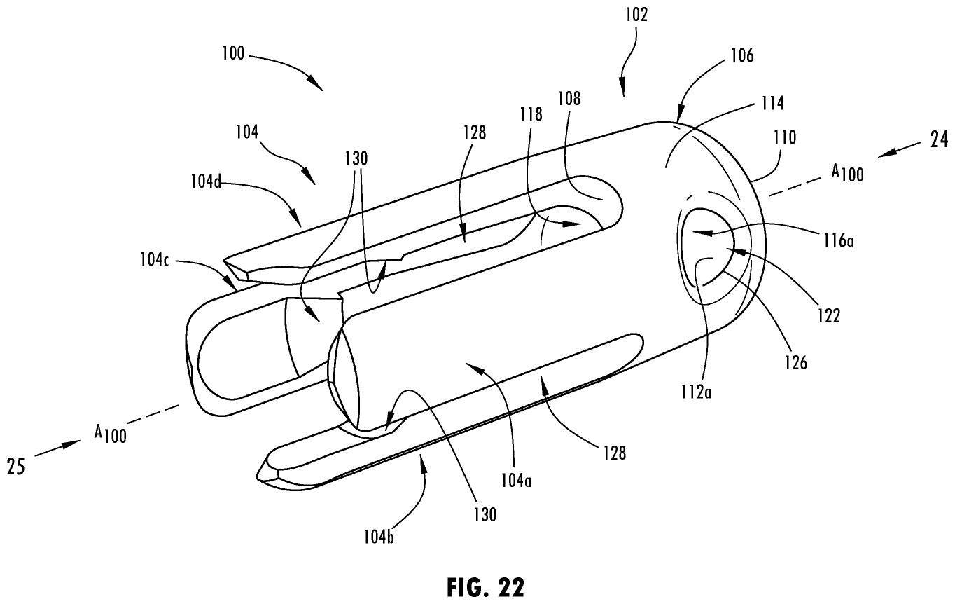

With reference to FIG. 29, the inner surface 112 of the body 106 of the head portion 102 of the cannula carrier 100 defines an axial passage 116. Access to the axial passage 116 is permitted by a proximal opening 118 (see, e.g., FIGS. 22, 25, and 27A-27H) and a distal opening 120 (see, e.g., FIGS. 18-21, 23, and 24). Furthermore, the inner surface 112 includes a first inner surface portion 112a defining a first passage portion 116a. The inner surface 112 also includes a second inner surface portion 112b defining a second passage portion 116b that is in fluid communication with the first passage portion 116a. The first inner surface portion 112a extends from the proximal end surface 108 and defines the proximal opening 118. The second inner surface portion 112b extends from the distal end surface 110 and defines the distal opening 120.

The first passage portion 116a is defined by a first passage diameter D₁₁₆ₐ (see, e.g., FIG. 29) and the second passage portion 116b is defined by a second passage diameter D_{116b} (see, e.g., FIG. 29). The first passage diameter D₁₁₆ₐ is greater than the second passage diameter D_{116b}. The second passage diameter D_{116b} is approximately equal to but slightly greater than the outer diameter D₁₂ of the cannula 12 so that at least a portion of the second passage portion 116b defined by the second inner surface portion 112b of the inner surface 112 of the body 106 of the head portion 102 of the cannula carrier 100 is configured to receive the cannula 12. As will be described in the following disclosure at FIGS. 28-31, upon arranging the cannula 12 within the second passage portion 116b, a portion L₁₂ₐ₁ of the length L₁₂ of the cannula 12 see, FIG. 2) may be arranged within the second passage portion 116b of the head portion 102 of the cannula carrier 100 such that the outer surface 22 of the tube-shaped body 16 of cannula 12 may be friction-fit-coupled to the second inner surface portion 112b of the inner surface 112 of the body 106 of the head portion 102 of the cannula carrier 100 in order to “plug” or “fluidly seal” the distal opening 120 of the cannula carrier (100).

Referring to FIGS. 18-20, 22, 23, 26A-26C, and 26G-26H, the body 106 of the head portion 102 of the cannula carrier 100 also defines a radial passage 122 that extends through the thickness T₁₀₆ body 106 of the head portion of the cannula carrier 100. As seen at FIG. 29, access to the radial passage 122 is provided by an inner surface opening 124 defined by the inner first inner surface portion 112a of the inner surface 112 of the body 106 of the cannula carrier 100 (i.e., the radial passage 122 is in direct fluid communication with the first passage portion 116a. With reference to any of FIGS. 18-20, 22, 23, 26A-26C, and 26G-26H, access to the radial passage 122 is also provided by an outer surface opening 126 defined by the outer surface 114 of the body 106 of the head portion 102 of the cannula carrier 100. As seen at, for example, FIG. 19, the radial passage 122 may be axially aligned with a leg portion 104, e.g., the first leg portion 104a of the plurality of leg portions 104, of the cannula carrier 100.

Each leg portion 104a, 104b, 104c, 104d axially extends from the proximal end surface 108 of the body 106 of the head portion 102 of the cannula carrier 100. Collectively, the leg portions 104a, 104b, 104c, 104d circumscribe a central axis A₁₀₀-A₁₀₀ extending through an axial center of the cannula carrier 100 and each may be circumferentially offset approximately 90° from an adjacent leg portion 104a, 104b, 104c, 104d, defining an axial gap 128 between each adjacent leg portion 104a, 104b, 104c, 104d.

Furthermore, with reference to, for example, FIGS. 18-23, each leg portion 104a, 104b, 104c, 104d is defined by a proximal barb portion 130. As seen at FIG. 29, each proximal barb portion 130 is defined by a plurality of barb surface portions 130a, 130b, 130c, 130d that may be configured to matingly-couple with the surface portions 65a, 65b, 65c, 65d that define the circumferential notch or groove 65 that extends into the outer body surface portion 62 of the outer surface 40 of the substantially tube-shaped body 34 of the hub 14. Each leg portion 104a, 104b, 104c, 104d may extend in a proximal direction beyond each barb surface portion 130a, 130b, 130c, 130d such that each leg portion 104a, 104b, 104c, 104d provides additional engagement with each rib 60a, 60b, 60c, 60d in order to transmit torque between the needle carrier 100 and the hub 14 (e.g., in order to engage and disengage the Luer lock mechanism).

As seen at FIGS. 28 and 29, a portion of the cannula 12 including the proximal end surface 18 at the proximal end 16_{P} of the tube-shaped body 16 is shown arranged near the distal opening 120 formed by the distal end surface 110 of the body 106 of the head portion 102 of the cannula carrier 100. The central axis A₁₂-A₁₂ (see, e.g., FIG. 2) of the cannula 12 is axially aligned with the central axis A₁₀₀-A₁₀₀ of the cannula carrier 100. The central axes A₁₂-A₁₂ and A₁₀₀-A₁₀₀ of each of the cannula 12 and the cannula carrier 100 correspond to the central axis A₁₀-A₁₀ (see FIG. 1) of the hypodermic interface assembly 10.

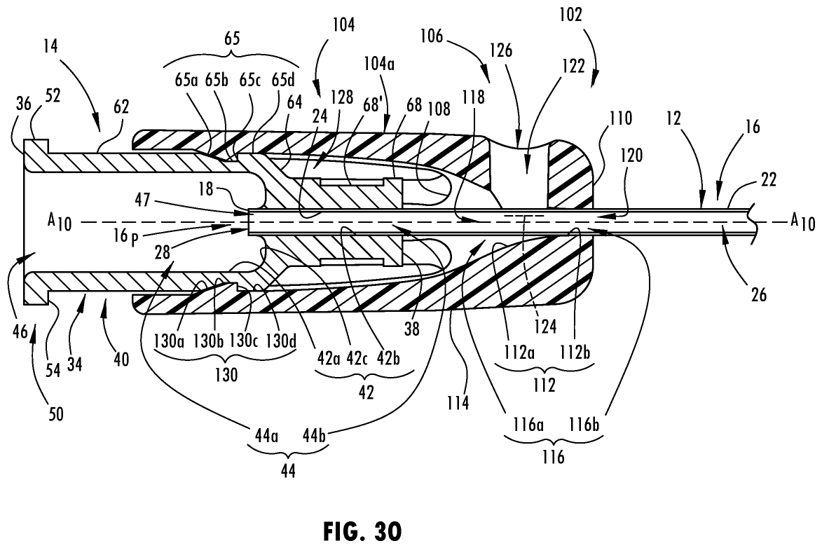

As described above, the outer surface 22 of the tube-shaped body 16 of the cannula 12 defines an outer diameter D₁₂ of the cannula 12, and the second passage diameter D_{116b} that defines the second passage portion 116b of the passage 116 is approximately equal to but slightly greater than the outer diameter D₁₂ of the cannula 12 so that at least a portion of the second passage portion 116b of the passage 116 defined by the body 106 of the cannula carrier 100 that is configured to receive the cannula 12 may result in the portion L₁₂ₐ₁ of the length L₁₂ tube-shaped body 16 of the cannula 12 “plugging” or “fluidly sealing” the distal opening 120 of the cannula carrier 100. Accordingly, as seen at FIGS. 29-30, the distal end 16_{D} of the tube-shaped body 16 of the cannula 12 is inserted (according to the direction of the arrow Y as seen at FIG. 29) through the proximal opening 118 formed by the proximal end surface 108 of the body 106 of the head portion 102 of the cannula carrier 100 and into the first passage portion 116a of the passage 116 for subsequent disposal within the second passage portion 116b of the passage 116.

Thereafter, the distal end 16_{D} of the tube-shaped body 16 of the cannula 12 is inserted through the distal opening 120 of the cannula carrier 100 such that a portion L_{12c} of the length L₁₂ of the tube-shaped body 16 of the cannula 12 extends beyond the distal end surface 110 of the body 106 of the head portion 102 of the cannula carrier 100. Advancement of the tube-shaped body 16 of the cannula 12 through the distal opening 120 of the cannula carrier 100 continues until the proximal barb portion 130 of each leg portion 104a, 104b, 104c, 104d of the plurality of leg portions 104 slides and flexes over the outer shoulder surface portion 64 of the outer surface 40 of the substantially tube-shaped body 34 of the hub 14 and then slides past and then flexes into for subsequent registration within the circumferential notch or groove 65. Once each leg portion 104a, 104b, 104c, 104d of the plurality of leg portions 104 is registered within the circumferential notch or groove 65, the plurality of barb surface portions 130a, 130b, 130c, 130d of each proximal barb portion 130 of each leg portion 104a, 104b, 104c, 104d of the plurality of leg portions 104 are disposed adjacent and matingly-coupled with the surface portions 65a, 65b, 65c, 65d that define the circumferential notch or groove 65 in order to mechanically couple the cannula carrier 100 to the hub 14.

With reference to FIG. 31, after the cannula carrier 100 is mechanically coupled to the hub 14, an amount of adhesive 200 is disposed within the first passage portion 116a of the passage 116 defined by the body 106 of the cannula carrier 100 for non-removably-coupling the cannula 12 to the cannula carrier 100. The adhesive 200 may be deposited or injected into the first passage portion 116a of the passage 116 defined by the body 106 of the cannula carrier 100 by way of the radial passage 122 that extends through the thickness T₁₀₆ body 106 of the head portion of the cannula carrier 100. In some instances, the radial passage 122 may be sized to receive a nozzle of an adhesive applicator A (as seen at, e.g., FIG. 31). Although the cannula carrier 100 is described above to be adhesively coupled to the cannula 12 with the adhesive 200, the cannula 100 could alternatively and/or additionally mechanically coupled to the cannula 12 by way of, for example, a material deformation process associated with, for example, swaging, crimping, welding, or the like. Exemplary welding procedures may include electron beam welding, ultrasonic welding, or the like.

As seen at FIG. 32, the adhesive 200 may fill at least a portion of, or all of, the first passage portion 116a of the passage 116 defined by the body 106 of the cannula carrier 100. In other configurations, the adhesive 200 may also fill at least a portion or all of the radial passage 122.

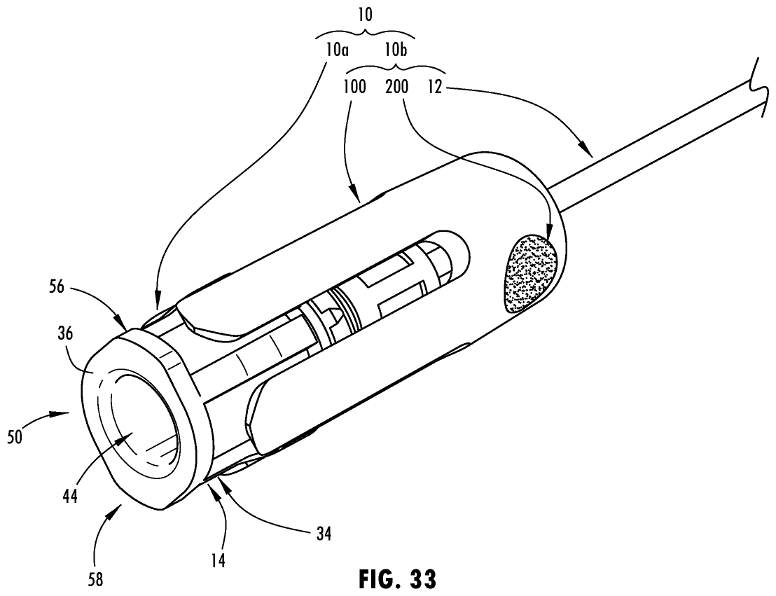

Once the desired amount of adhesive 200 is deposited into the first passage portion 116a, the adhesive 200 may surround and be disposed adjacent: (1) a portion L₁₂ₐ₁ of the length L₁₂ defined by the outer surface 22 of the tube-shaped body 16 of the cannula 12 that is arranged within the first passage portion 116a; and (2) at least a portion of the first inner surface portion 112a that defines the first passage portion 116a for non-removably and adhesively-joining the cannula 12 to the cannula carrier 100. In some configurations, the adhesive 200 is an acrylic adhesive, a cyanoacrylate adhesive, a ultra-violet (UV) curable adhesive, epoxy resin, urethane resin, polyester resin, alkyd resin, or the like. As seen at FIG. 32, if the adhesive 200 is a UV curable adhesive, a UV light source (not shown) may be utilized for directing ultra violet light UV toward the adhesive 200 for curing the adhesive 200. Once the adhesive 200 has cured, the hypodermic interface assembly 10 may be said to be formed as seen at FIGS. 33-35B.

Although the exemplary design of the hypodermic interface assembly 10 utilizes both of: (1) the proximal barb portion 130 of each leg portion 104a, 104b, 104c, 104d to mechanically-join the cannula carrier 100 to the hub 14; and (2) adhesive 200 for adhesively-joining cannula carrier 100 to the cannula 12, some implementations of the hypodermic interface assembly 10 may include one of a mechanical coupling and/or an adhesive coupling. For example, the hypodermic interface assembly 10 may be only mechanically-joined by friction-fit connecting the cannula 12 to the cannula carrier 100 (i.e., the adhesive 200 may be excluded from such an exemplary implementation of a hypodermic interface assembly) and the proximal barb portion 130 of each leg portion 104a, 104b, 104c, 104d.

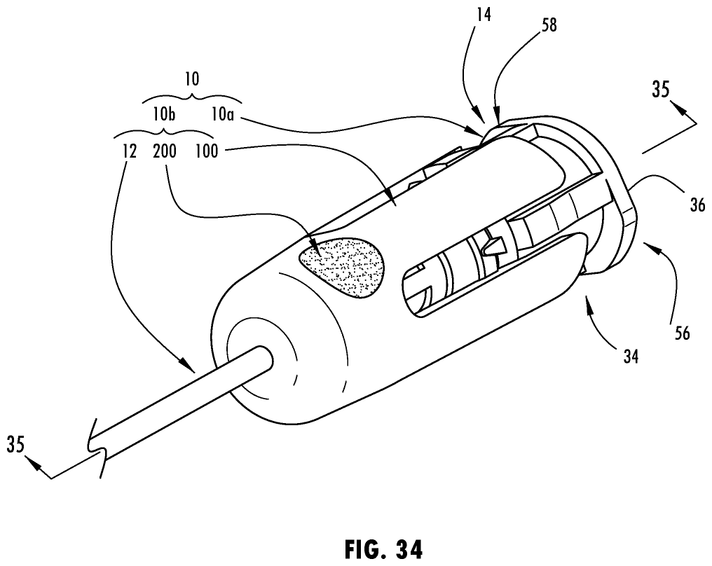

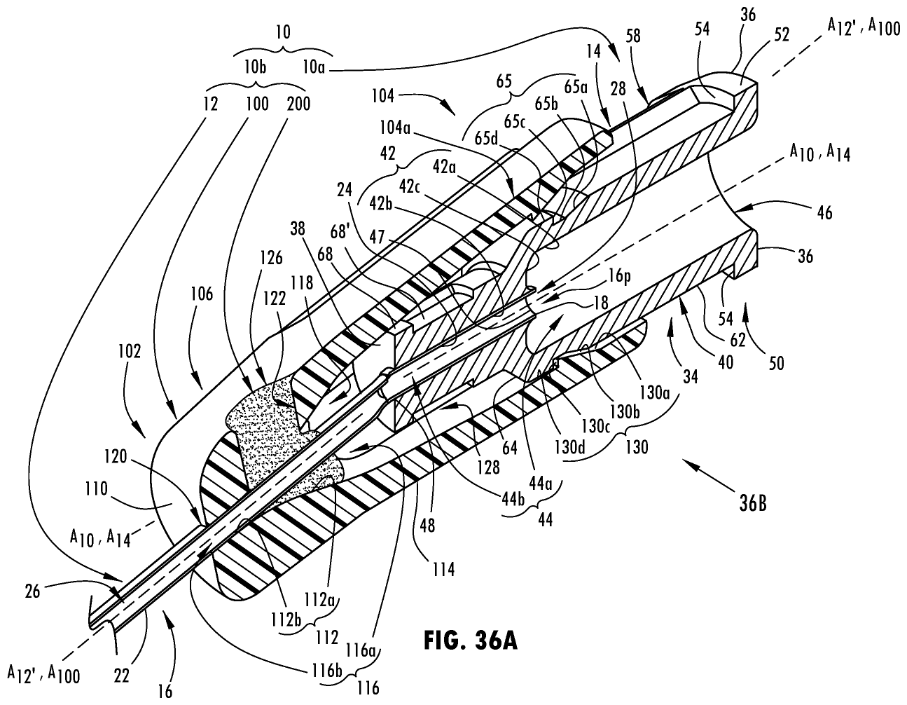

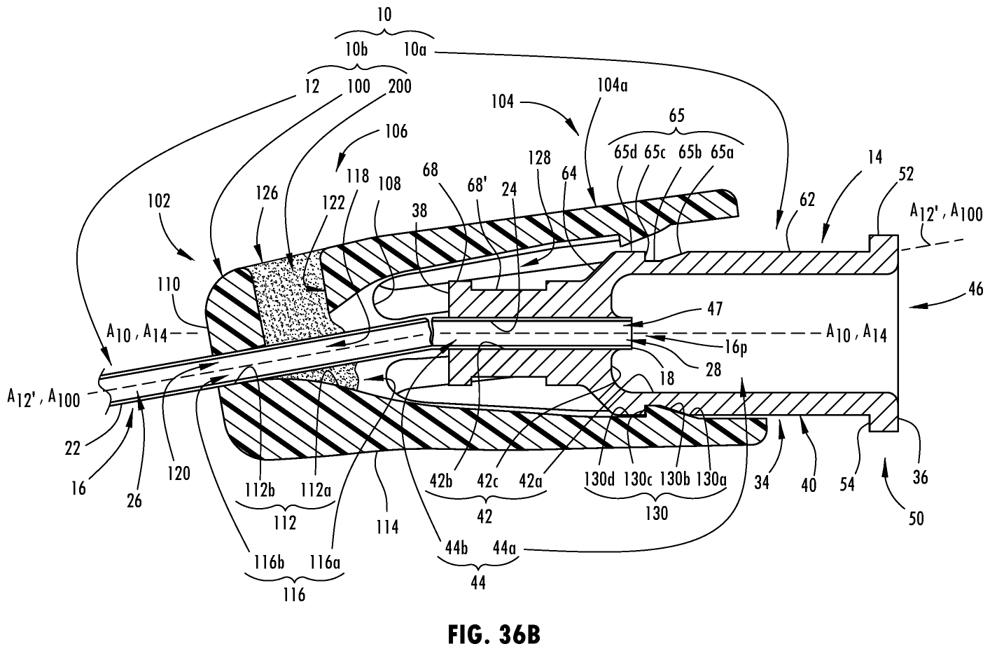

Referring to FIGS. 34 and 35A-35B, the hypodermic interface assembly 10 is shown at an at-rest orientation. At FIGS. 36A-36B, the hypodermic interface assembly 10 is shown at a biased orientation. Thereafter, as seen at FIGS. 37A-37B, the hypodermic interface assembly 10 is shown arranged in a separated orientation defined by a first portion 10a of the hypodermic interface assembly 10 that is configured to remain attached to an injection gun I and a second portion 10b of the hypodermic interface assembly 10 that is configured to be removed from an impaled orientation within the flesh of the animalia S.

With reference to FIG. 38A, the third inner surface portion 42c of the inner surface 42 of the substantially tube-shaped body 34 of the hub 14 may define a curved or frustoconical surface that extends into the first passage portion 44a of the passage 44 of the hub 14. As seen at FIG. 38B, a peak of the curved or frustoconical surface defined by the third inner surface portion 42c of the inner surface 42 of the substantially tube-shaped body 34 is defined generally by a dashed line P1 that is orthogonal to the central axis A₁₀-A₁₀ of the hypodermic interface assembly 10. Furthermore, as also seen at FIG. 38B, another dashed line S1 that is orthogonal to the central axis A₁₀-A₁₀ of the hypodermic interface assembly 10 extends across the region where the proximal barb portion 130 of each leg portion 104a, 104b, 104c, 104d mechanically-joins the cannula carrier 100 to the circumferential notch or groove 65 formed by a portion of the outer body surface portion 62 of the outer surface 40 of the substantially tube-shaped body 34 of the hub 14. Yet even further, another dashed line B1 that is orthogonal to the central axis A₁₀-A₁₀ of the hypodermic interface assembly 10 extends across the distal end surface 38 of the hub 14. Further, yet another dashed line A1 that is orthogonal to the central axis A₁₀-A₁₀ of the hypodermic interface assembly 10 extends across a region of the head portion 102 of the cannula carrier 100 near the proximal end surface 108 of the body 106 of the head portion 102 of the cannula carrier 100.

The dashed line S1 generally demarcates a region of the hypodermic interface assembly 10 where the proximal barb portion 130 of each leg portion 104a, 104b, 104c, 104d of the cannula carrier 100 is configured to predictably mechanically separate from surface portions 65a, 65b, 65c, 65d that define the circumferential notch or groove 65 such that the cannula carrier 100 is permitted to mechanically separate from the hub 14. Furthermore, the dashed line B1 generally demarcates a region of the hypodermic interface assembly 10 where the cannula 12 may (but is not intended to) structurally fail and break into first and second portions. As such, a portion of the cannula 12 and the hub 14 that defines the first portion 10a of the hypodermic interface assembly 10 is configured to remain attached to the injection gun I. Similarly, the other portion of the cannula 12 that is non-removably-joined to the cannula carrier 100 by the adhesive 200 defines the second portion 10b of the hypodermic interface assembly 10 is configured to be removed from an impaled orientation within the flesh of the animalia S. Yet even further, the dashed line A1 generally demarcates a “fill line” where the amount of adhesive 200 should not further fill the first passage portion 116a or surround the cannula 12 in a region beyond the “fill line” A1. Furthermore, a distance extending between the “fill line” A1 and the dashed line B1 may be sized to permit the cannula 12 to in the region between the dashed lines A1 and B1 while not allowing the any surface portion of the needle carrier 100 to engage or come into contact with the distal end surface 38 of the hub 14.

Although the structural integrity of the cannula 12 is shown to potentially (but is not intended to) fail in association with the exemplary implementation of the hypodermic interface assembly 10 as seen at FIGS. 37A-37B and 38A-38B, resulting in the cannula 12 breaking into first and second portions as seen at FIGS. 37A and 37B, with reference to FIGS. 38C and 39A-39C, the proximal end 16_{P} of the tube-shaped body 16 of the cannula 12 may be arranged within the hub 14 closer to the distal end surface 38 of the hub 14. Furthermore, as seen at FIG. 38C, the proximal end 16_{P} of the tube-shaped body 16 of the cannula 12 may be arranged proximal of a dashed line P2 that extends across a proximal-most end of the crimping pockets 68′ defined by the outer head surface portion 68 of the outer surface 40 of the substantially tube-shaped body 34 of the hub 14. Although the proximal end 16_{P} of the tube-shaped body 16 of the cannula 12 is shown arranged proximal of the dashed line P2, the proximal end 16_{P} of the tube-shaped body 16 of the cannula 12 may be arranged anywhere between, for example the dashed line B1 and the dashed line P2.

With reference to FIG. 38D, another exemplary configuration of the hypodermic interface assembly 10 is shown that is substantially similar to FIG. 38C with the exception that the body 106 of the head portion 102 of the cannula carrier 100 includes an adhesive blocking wall 132 including a cannula passage 134. The adhesive blocking wall 132 contains the adhesive 200 within at least a portion of the first passage portion 116a and prevents axial migration of the adhesive 200 in a direction further toward the dashed line B1 such that the adhesive 200 does not surround a portion of the cannula extending beyond the dashed line A1.

With reference to FIG. 38E, another exemplary configuration of the hypodermic interface assembly 10 is shown that is substantially similar to FIG. 38C with the exception that second passage portion 116b is arranged at an angle θ_{116b} that is not orthogonal to the central axis A₁₀-A₁₀. In an example, the angle θ_{116b} may be approximately equal to about 45° in order to assist in controlled depositing of the adhesive 200 into the first passage portion 116a such that the adhesive 200 substantially surrounds the cannula 12 up to and not beyond the fill line A1.

With reference to FIG. 38F, another exemplary configuration of the hypodermic interface assembly 10 is shown that is substantially similar to FIG. 38E with the exception that the body 106 of the head portion 102 of the cannula carrier 100 includes the adhesive blocking wall 132 including a cannula passage 134. The adhesive blocking wall 132 contains the adhesive 200 within at least a portion of the first passage portion 116a and prevents axial migration of the adhesive 200 in a direction further toward the dashed line B1 such that the adhesive 200 does not surround a portion of the cannula extending beyond the dashed line A1. Such an arrangement seen at FIGS. 38C and 39A results in the proximal end 16_{P} of the tube-shaped body 16 of the cannula 12 being “ripped” out of, or, alternatively, being “migrated away from” (as seen at FIGS. 39B and 39C) the first portion 10a of the hypodermic interface assembly 10 while the entire tube-shaped body 16 of the cannula 12 remains joined to the second portion 10b of the hypodermic interface assembly 10. Accordingly, as seen at FIG. 39C, all of the cannula 12 remains structurally intact and does not break into first and second portions as seen at FIGS. 37A and 37B (and, as a result, does not define a ‘break line’).

Referring now to FIGS. 40 and 41A-41G, a methodology for utilizing any configuration of the exemplary hypodermic interface assemblies 10 is shown. Although FIGS. 40 and 41A-41G show a methodology for utilizing the hypodermic interface assembly 10 described at FIGS. 33-35B, any of the other hypodermic interface assemblies described in the present disclosure may also be utilized in a substantially similar manner as seen at FIGS. 40 and 41A-41G.

As described above, the design of the hypodermic interface assembly 10 promotes controlled separation (see, e.g., FIGS. 37A-37B, 39A-39C and 41E) of the cannula 12, the cannula carrier 100, and the adhesive 200 (that collectively define the second portion 10b of the hypodermic interface assembly 10) relative to the hub 14 (that defines the first portion 10a of the hypodermic interface assembly 10).

In some instances, predictable and controlled separation of the second portion 10b of the hypodermic interface assembly 10 from the first portion 10a of the hypodermic interface assembly 10 may occur after the cannula 12 pierces the subject S (see, e.g., FIGS. 41A-41B). The subject S may be, for example, animalia, such as a human or non-human (i.e., an animal such as, for example, pig or swine). In other examples, the subject S may be an inanimate object. The predicable and controlled separation of the second portion 10b of the hypodermic interface assembly 10 from the first portion 10a of the hypodermic interface assembly 10 mitigates separation of the cannula 12 from the entirety of the hub 14, which may otherwise undesirably result in the cannula 12 being broken-off and subsequently lost (or make difficult easily locating the broken-off cannula) within the flesh of the animalia.

Referring to FIG. 40, the hypodermic interface assembly 10 is shown connected to an injecting device I, such as, for example, an injection gun. The hypodermic interface assembly 10 may be connected to a barrel portion I_{B} of the injection gun I by arranging, for example, the first radially-outward projection or ear 56 and the second radially-outward projection or ear 58 extending from the of the barrel-engaging portion 50 that extends from the outer surface 40 of the substantially tube-shaped body 34 of the hub 14 in corresponding recesses (not shown) formed by the barrel portion I_{B} of the injection gun I and then, for example, quarter-turn locking the hypodermic interface assembly 10 for removably-securing the first radially-outward projection or ear 56 and the second radially-outward projection or ear 58 extending from the of the barrel-engaging portion 50 to the barrel portion I_{B} of the injection gun I.

The injection gun I may include a fluid container C that contains a fluid F (see also, e.g., FIG. 41C). The fluid F may be metered from: (1) the container C; (2) through the injection gun I; (3) into the hypodermic interface assembly 10; and (4) out of the hypodermic interface assembly 10 and into the flesh of the subject S. The injection gun I may be actuated when a user U presses, for example, an actuator IA such as, for example, a trigger in order to cause movement of the fluid F as described above. The injection gun I may be powered in any desirable manner such as, for example: battery powered; air powered; manually powered; or a combination thereof.

Referring to FIG. 41A, the user may grasp the injection gun I and position the sharp piercing tip 32 formed by the distal end surface 20 of the tube-shaped body 16 of the cannula 12 near the outer surface S_{S} of the subject S, which may define the skin or hide of the subject S. Referring to FIGS. 41A-41B, the user U may impart an axial force according to the direction of the arrow X_{A} to the injection gun I along the central axis A₁₀-A₁₀ extending through the hypodermic interface assembly 10 such that the sharp piercing tip 32 formed by the distal end surface 20 of the tube-shaped body 16 of the cannula 12 axially pierces the outer surface S_{S} of the subject S.

Referring to FIGS. 38A and 41C, after the outer surface S_{S} of the subject S has been axially pierced by the cannula 12, the user U may optionally actuate the actuator IA in order to cause movement of the fluid F from: (1) the container C; (2) through the injection gun I; (3) into the hypodermic interface assembly 10; and (4) out of the hypodermic interface assembly 10 and into the flesh of the subject S. In an example, the fluid F may firstly enter the hypodermic interface assembly 10 from the injection gun I at the passage 44 formed by the substantially tube-shaped body 34 of the hub 14 by way of the proximal opening 46 formed by the proximal end surface 36 of the substantially tube-shaped body 34 of the hub 14. Then, the fluid F may secondly enter the passage 26 extending through the tube-shaped body 16 of the cannula 12 by way of the proximal opening 28 formed by the proximal end surface 18 of the body 16 of the cannula 12. Then, thirdly, the fluid F may exit the passage 44 formed by the substantially tube-shaped body 34 of the hub 14 by way of the distal opening 48 formed by the distal end surface 38 of the substantially tube-shaped body 34 of the hub 14. Thereafter, fourthly, the fluid F may exit the passage 26 extending through the tube-shaped body 16 of the cannula 12 by way of the distal opening 30 formed by the distal end surface 20 of the body 16 of the cannula 12.

The fluid F may be any desirable composition that is intended to be delivered to the animalia S. In some instances, the fluid F may be a medicament, a pharmaceutical, a vaccine, an anesthetic, or the like. Accordingly, the fluid F may not include any type of fluid that is not intended to be injected into animalia S. Although the hypodermic interface assembly 10 also may be utilized for injecting fluid F into animalia S, the hypodermic interface assembly 10 may be utilized for removing fluid F (e.g., blood) from animalia S. Therefore, it will be appreciated that the hypodermic interface assembly 10 may deliver or receive fluid F.

Referring to FIGS. 36A-36B, 39B, and 41D, after the outer surface S_{S} of the subject S has been axially pierced by the cannula 12, the subject S may experience discomfort as a result of pain arising from the outer surface S_{S} being pierced by the sharp piercing tip 32 formed by the distal end surface 20 of the tube-shaped body 16 of the cannula 12. Accordingly, if the user U is sufficiently grasping the injection gun I, any movement of the subject S may result in the cannula 12 being subjected to one or more radial forces X_{R} relative the central axis A₁₀-A₁₀ extending through the hypodermic interface assembly 10 that may cause the cannula 12 to bend or warp, such that the central axis A₁₂-A₁₂ extending through the axial center of the tube-shaped body 16 of the cannula 12 is not coincident with the central axis A₁₀-A₁₀ extending through the hypodermic interface assembly 10 that may be coaxially aligned with the other components of the hypodermic interface assembly 10 such as, for example, the hub 14 and the cannula carrier 100.

Because the cannula carrier 100 may be formed from a flexible or substantially non-rigid material (e.g., plastic), any stresses imparted to the cannula 12 arising from the one or more radial forces X_{R} may be transmitted from the cannula 12 to the cannula carrier 100; and any such stresses transmitted from the cannula 12 to the cannula carrier 100 may be directed to and concentrated at a predetermined portion or region of the hypodermic interface assembly 10. The predetermined portion or region of the hypodermic interface assembly 10 that receives the concentrated stresses is generally defined by a portion or region of the hypodermic interface assembly 10 where the separation line (see, e.g., the dashed line S1 of FIGS. 38B, 38C, which may be referred to as a separation line) traverses the cannula carrier 100 and the hub 14 of the hypodermic interface assembly 10. As described above, the separation line S1 generally demarcates the regions with the cannula carrier 100 being capable of mechanically separating from the hub 14. Also, depending on the inset orientation of the cannula 12 relative the hub 14, the cannula 12 may remain intact, unbroken (as seen at, e.g., FIG. 39C), or, in some configurations, the cannula 12 may (but is not intended to) structurally fail and break (as seen at, e.g., FIG. 37A and according to the break line B1 of FIG. 38B) as a result of a stress concentration arising from the one or more radial forces X_{R} that also may be transmitted to the cannula 12 and concentrated at, substantially at, about, along, or on the region of the hypodermic interface assembly 10 defined by the break line B1. And in either implementation, the distal portion of the cannula 12 remains non-removably connected to the cannula carrier 100 by way of, for example, the adhesive 200.

As seen at FIG. 38A, the cannula carrier 100 may be defined by a length L₁₀₀ that is further defined by sub-lengths L₁₀₀ₐ and L_{100b}. The sub-length L_{100b} may be further defined by sub-length portions L_{100b1} and L_{100b2}.

The sub-length L₁₀₀ₐ may be defined by a length of the head portion 102 of the cannula carrier 100. The sub-length L_{100b} may be defined by a length of each leg portion 104a, 104b, 104c, 104d of the cannula carrier 100. The sub-length portion L_{100b1} may be defined by a length of each leg portion 104a, 104b, 104c, 104d not including the proximal barb portion 130 of each leg portion 104a, 104b, 104c, 104d. The sub-length portion L_{100b2} may be defined by a length of each proximal barb portion 130 of each leg portion 104a, 104b, 104c, 104d.

Each length portion L₁₀₀ₐ and L_{100b} and each sub-length portions L_{100b1} and L_{100b2} may be selectively distanced or configured in order to optimize mechanical separation of the cannula carrier 100 from the hub 14 at the separation line S1. For example, sufficient leg length (e.g., as defined by the sub-length portion L_{100b2}) may assist in allowing each leg portion 104a, 104b, 104c, 104d of the cannula carrier 100 to separate from the hub 14 at the separation line S1 without the body 106 of cannula carrier 100 interfering with the distal end surface 38 of the hub 14 when the one or more radial forces X_{R} is/are applied to the cannula 12. Furthermore, a combination of the selective distancing or configuration of the length portions L₁₀₀ₐ and Limb and each sub-length portions L_{100b1} and L_{100b2} in combination with providing a UV adhesive for the adhesive portion 200 may strengthen the overall hypodermic interface assembly 10.

As seen at FIG. 41D, as a result of stresses transmitted from the cannula 12 to the hub 14 being directed to and concentrated at a predetermined portion or region of the cannula carrier 100 and the hub 14, the cannula carrier 100 may be permitted to also bend or deviate with the cannula 12 away from the central axis A₁₀-A₁₀ extending through the hypodermic interface assembly 10 (see, e.g., the axes A₁₂-A₁₂, A₁₀₀-A₁₀₀ of the cannula 12 and the cannula carrier 100). Accordingly, the axes A₁₂-A₁₂, A₁₀₀-A₁₀₀ of the cannula 12 and the cannula carrier 100 generally deviate away from the axis A₁₄-A₁₄ of the hub 14, which may remain coincident with the central axis A₁₀-A₁₀ extending through the hypodermic interface assembly 10.

Referring to FIGS. 37A-37B and 41E, the stresses transmitted from the cannula 12 to the interface assembly (10) that were directed to and concentrated at the predetermined portion or region (e.g., at separation line S1) may continue to bend the cannula carrier 100 relative the hub 14 until the cannula carrier 100 controllably mechanically separates from the hub 14 by flexibly-disconnecting the proximal barb portion 130 of each leg portion 104a, 104b, 104c, 104d of the cannula carrier 100 from surface portions 65a, 65b, 65c, 65d that define the circumferential notch or groove 65 such that the cannula carrier 100 is permitted to mechanically separate from the hub 14. As a result, the second portion 10b of the hypodermic interface assembly 10 including the cannula carrier 100, the cannula 12, and the adhesive 200 predictably and controllably separates from the first portion 10a of the hypodermic interface assembly 10 defined by the hub 14 (at, or substantially at, about, along, or on break line B1). After separation, each leg portion 104a, 104b, 104c, 104d of the cannula carrier 100 may deform, expand, or splay outwardly, which may increase visibility to a user to assist in locating where the cannula 12 is impaled within the flesh of the animalia S.

As seen at FIG. 41E, because the cannula carrier 100 is non-separably joined to the cannula 12 with the adhesive 200, the user U, may easily identify a location of the animalia S where the cannula 12 is impaled within the flesh of the animalia S. The location of the animalia S where the cannula 12 is impaled within the flesh of the animalia S is easily identifiable as a result of, for example, the cannula carrier 100 of the second portion 10b of the hypodermic interface assembly 10 resting upon the skin S_{S} or hide of the animalia S (while the cannula 12 is not visible to the user U since the cannula 12 is contained within and obscured by the flesh of the animalia S.

Thereafter, as seen at FIG. 41F, the user U may pinch or grasp the second portion 10b of the hypodermic interface assembly 10 and apply a pulling force to the second portion 10b of the hypodermic interface assembly 10 (that also includes the impaled cannula 12). As seen at FIG. 41G, as a result of the pulling force to the second portion 10b of the hypodermic interface assembly 10 by the user U, the cannula 12 is removed from the flesh of the animalia S such that the cannula 12 is not lost or would therefore otherwise undesirably remain within the flesh of the animalia S.

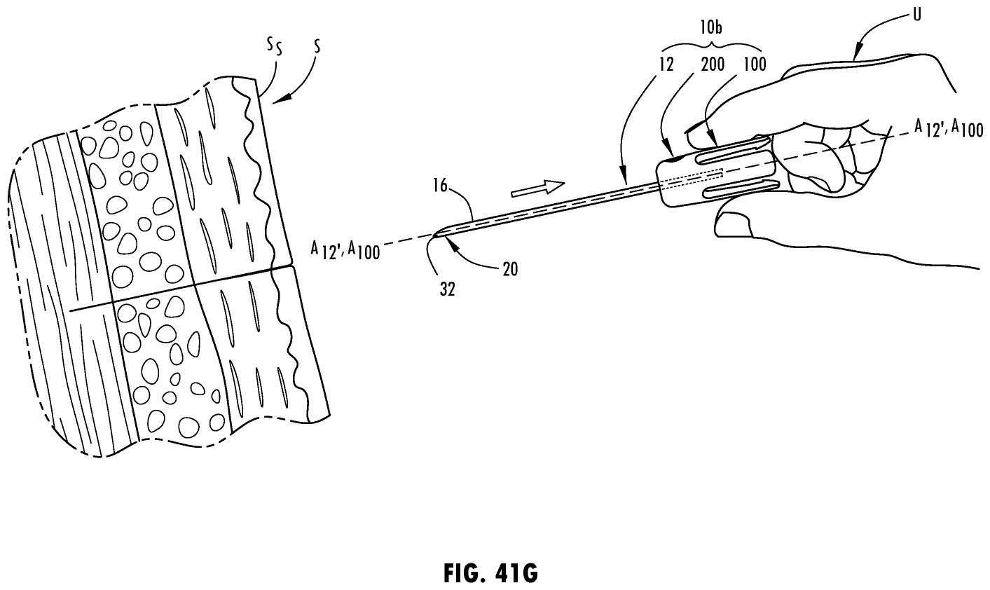

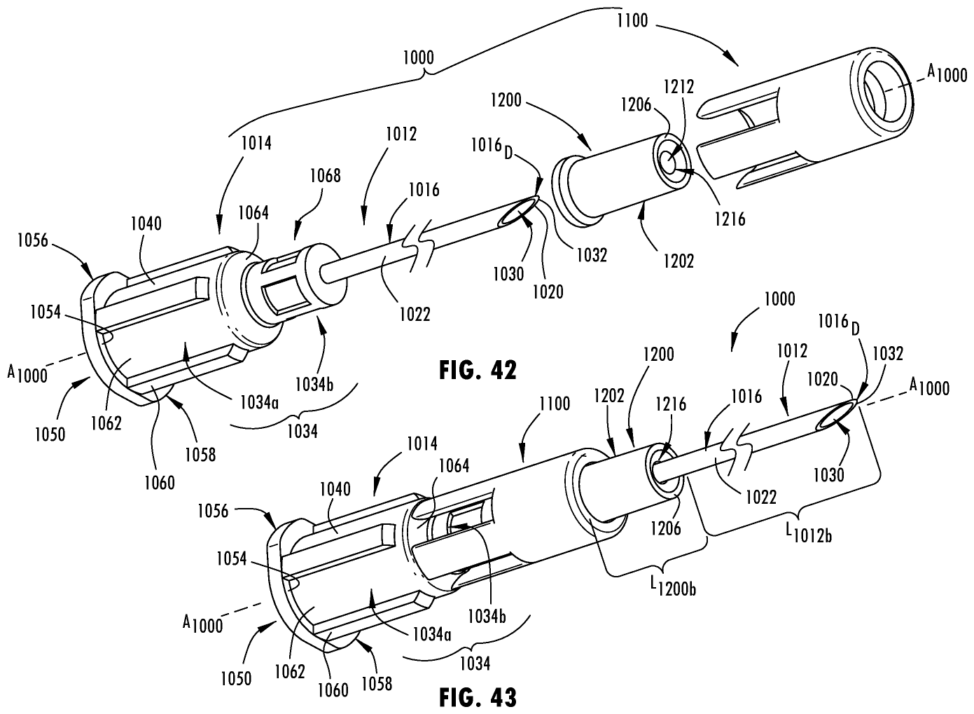

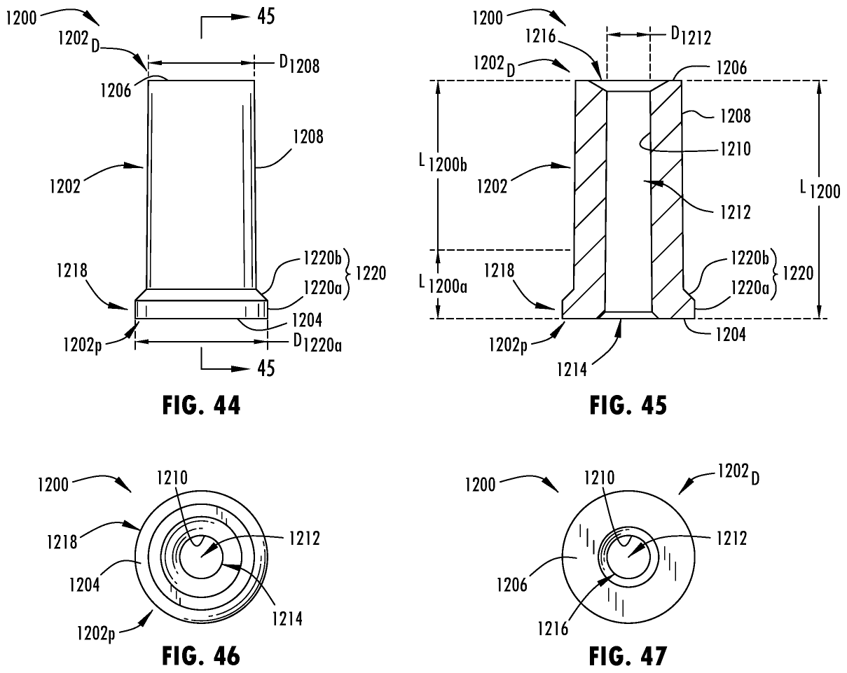

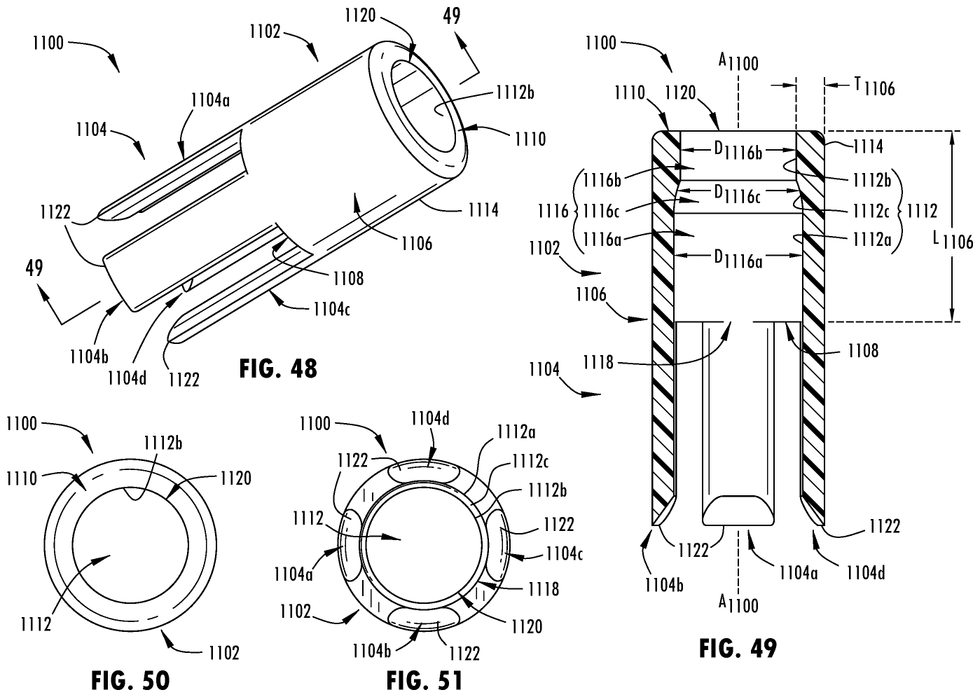

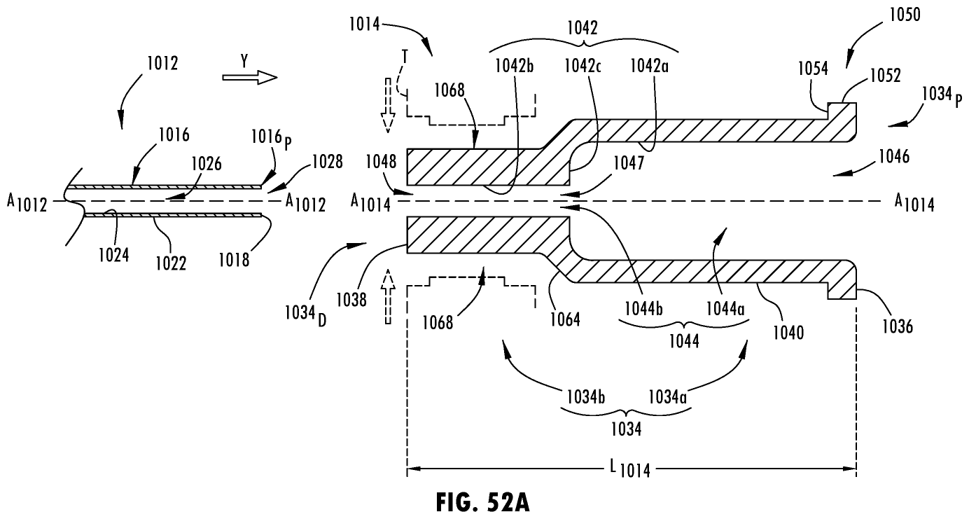

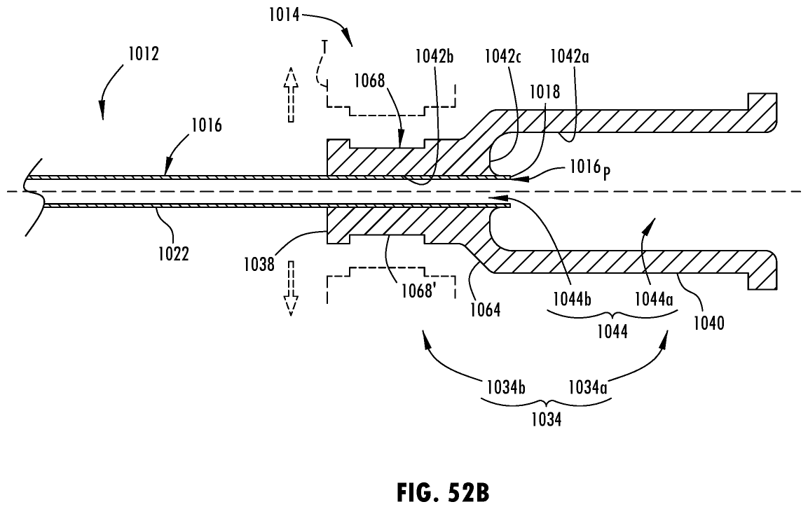

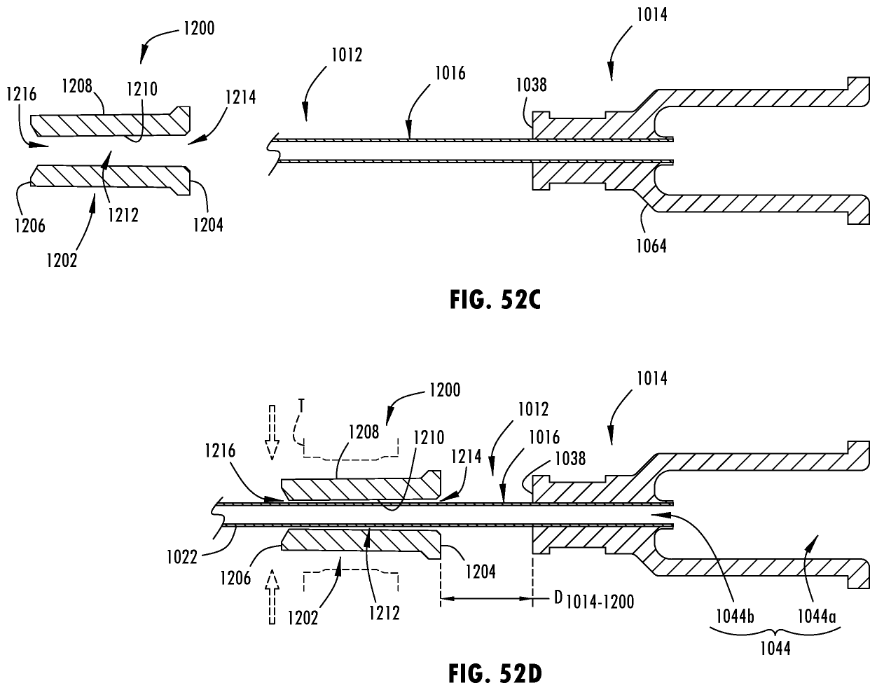

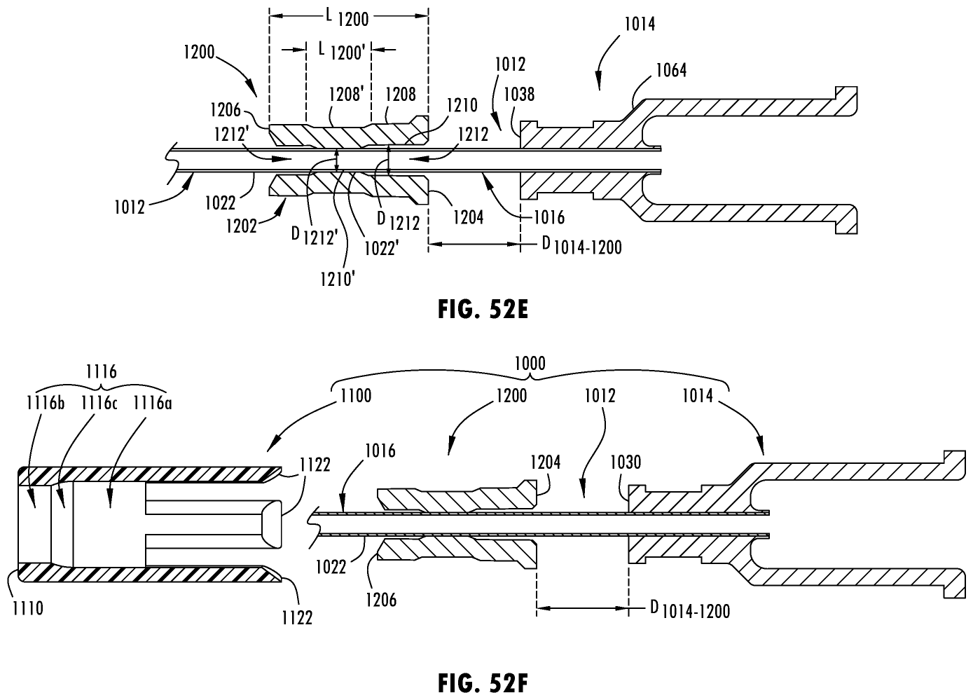

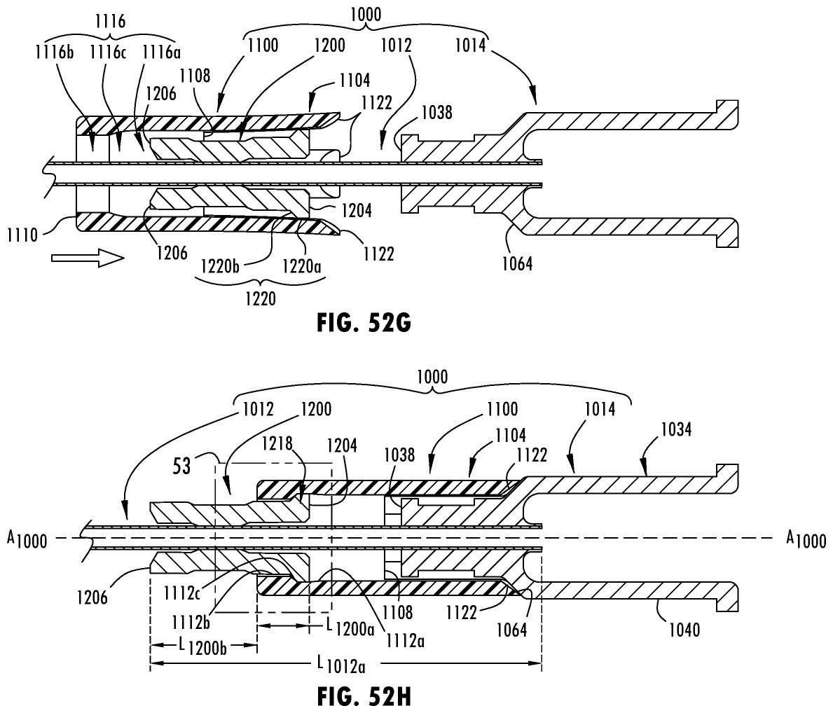

Referring to FIGS. 42-43, 52F-52H, 53, and 54A-54C, a hypodermic interface assembly is shown at 1000. Referring to FIGS. 42-43, the hypodermic interface assembly includes a cannula 1012, a hub 1014, a cannula carrier 1100 (see, e.g., FIGS. 48-51), and a core member 1200 (see, e.g., FIGS. 44-47). Furthermore, a first sub-assembly of the hypodermic interface assembly 1000 that is defined by the cannula 1012 and the hub 1014 (see, e.g., FIGS. 52A-52B). Even further, a second sub-assembly of the hypodermic interface assembly 1000 that is defined by the cannula 1012, the hub 1014, and the core member 1200 (see, e.g., FIGS. 52C-52E). Yet even further, a third sub-assembly of the hypodermic interface assembly 1000 that is defined by the cannula 1012, the hub 1014, the core member 1200, and the cannula carrier 1100 (see, e.g., FIGS. 52F-52H). With reference to FIGS. 42-43 and 52H, a central axis that extends through an axial center of each component (e.g., the cannula 1012, the hub 1014, the cannula carrier 1100, and the core member 1200) of the hypodermic interface assembly 1000 is shown generally at A₁₀₀₀-A₁₀₀₀.

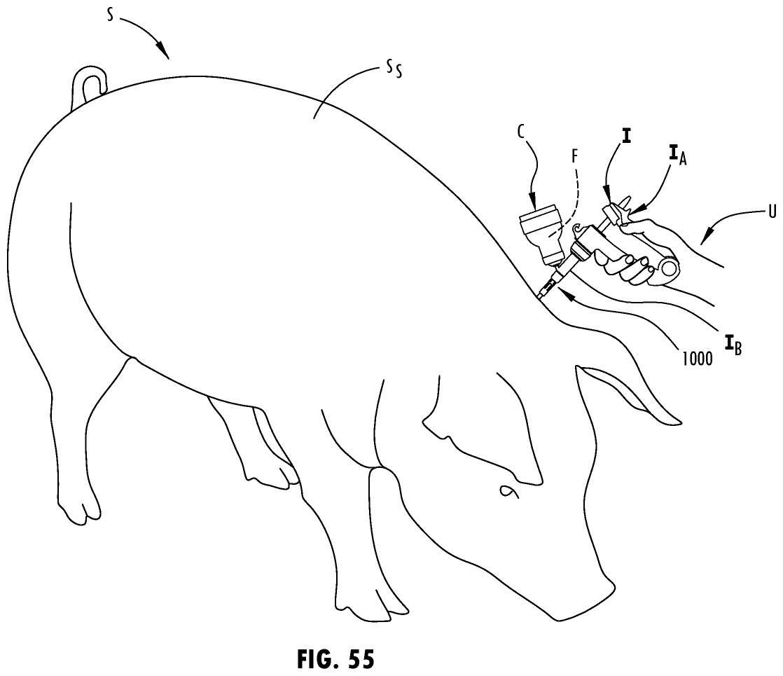

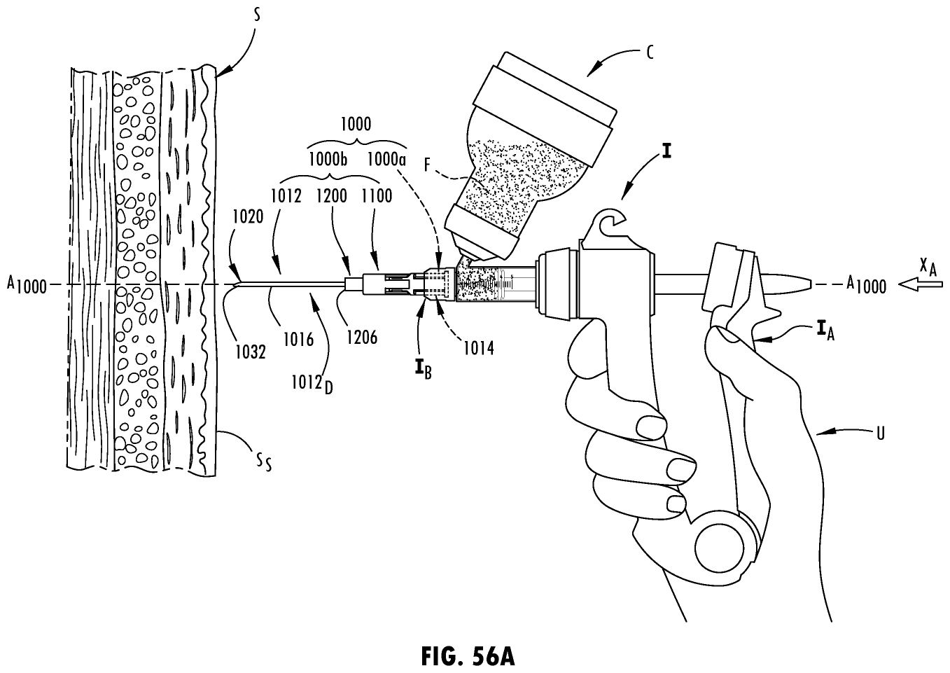

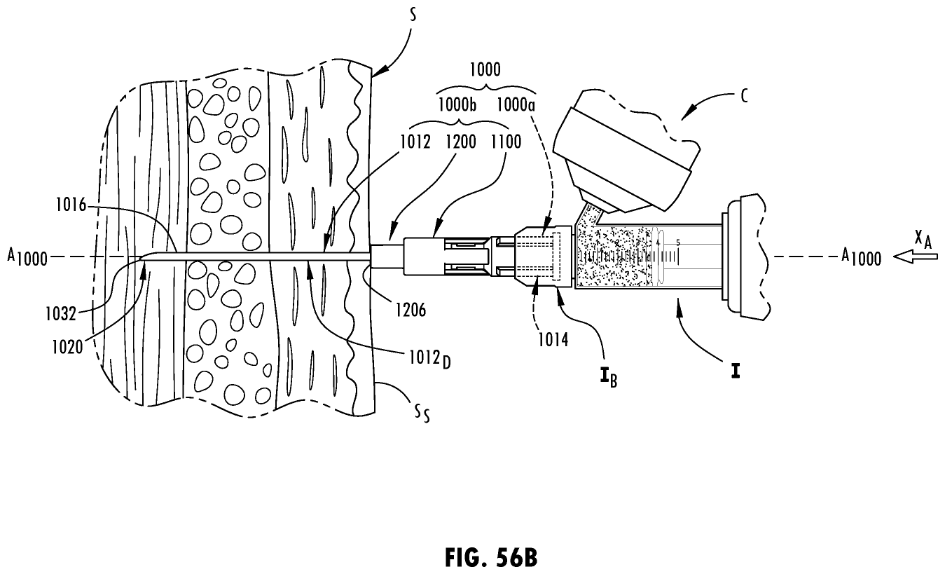

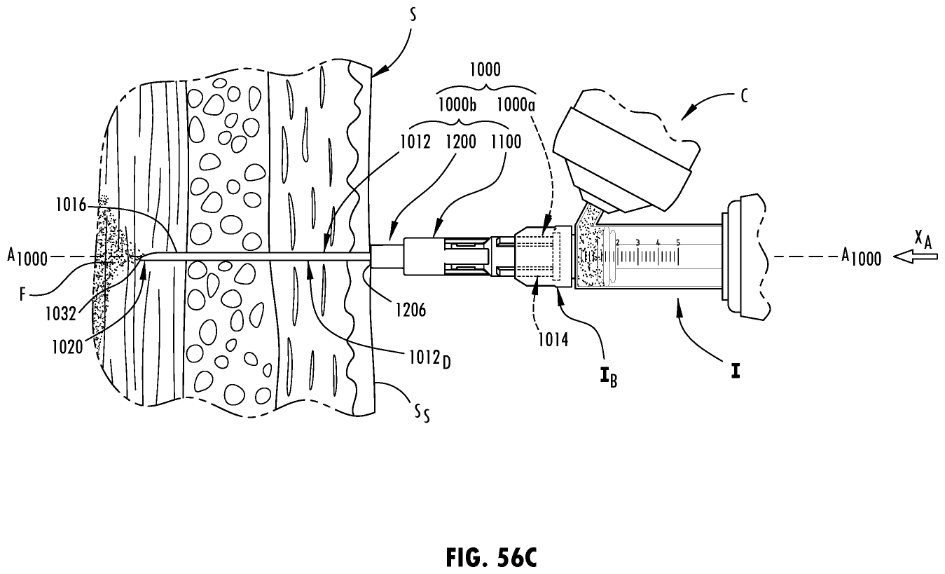

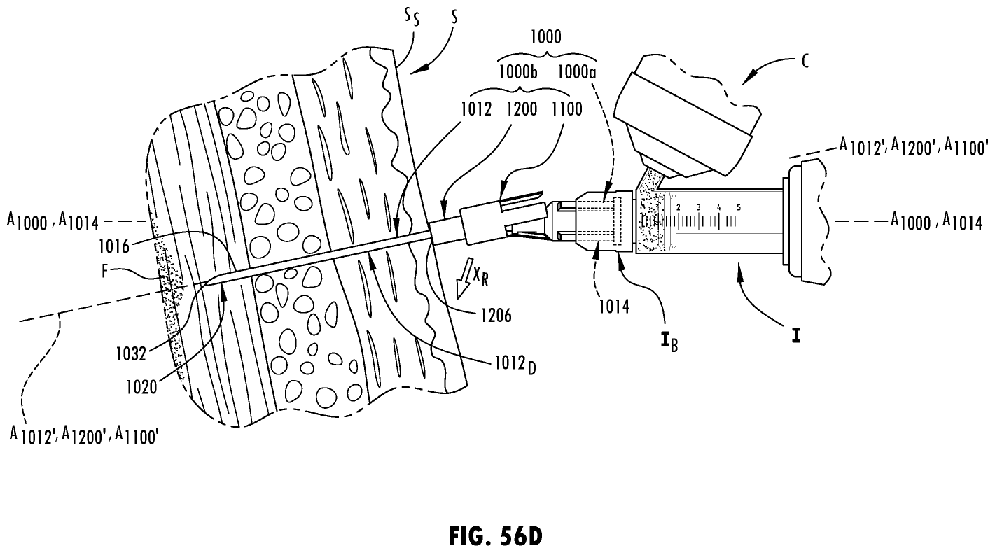

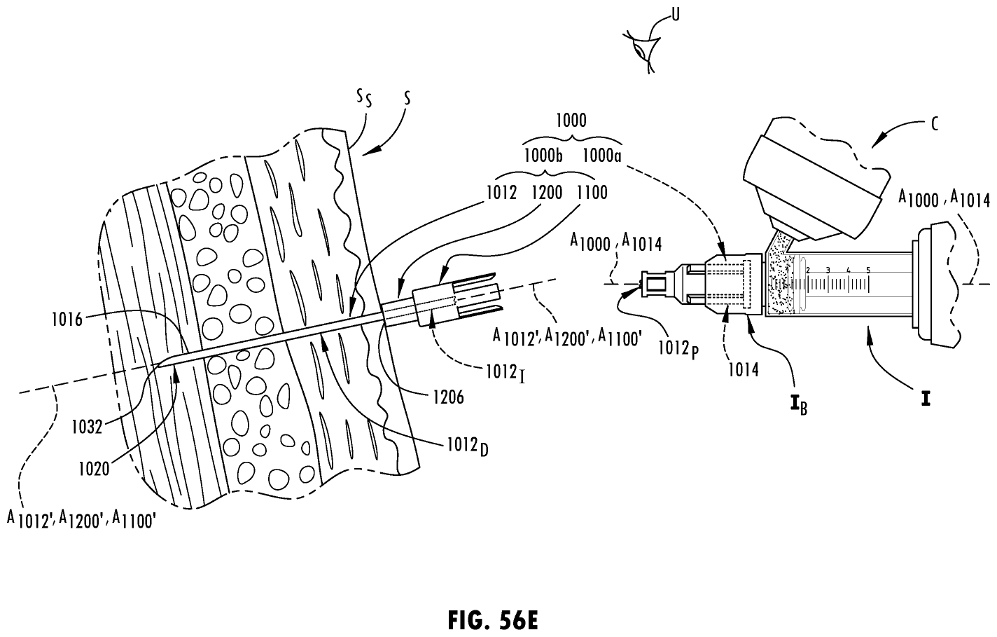

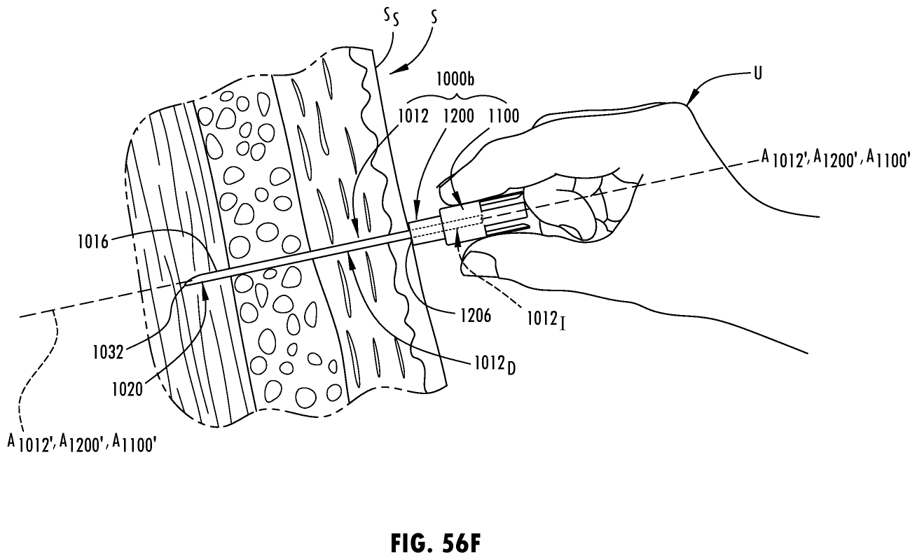

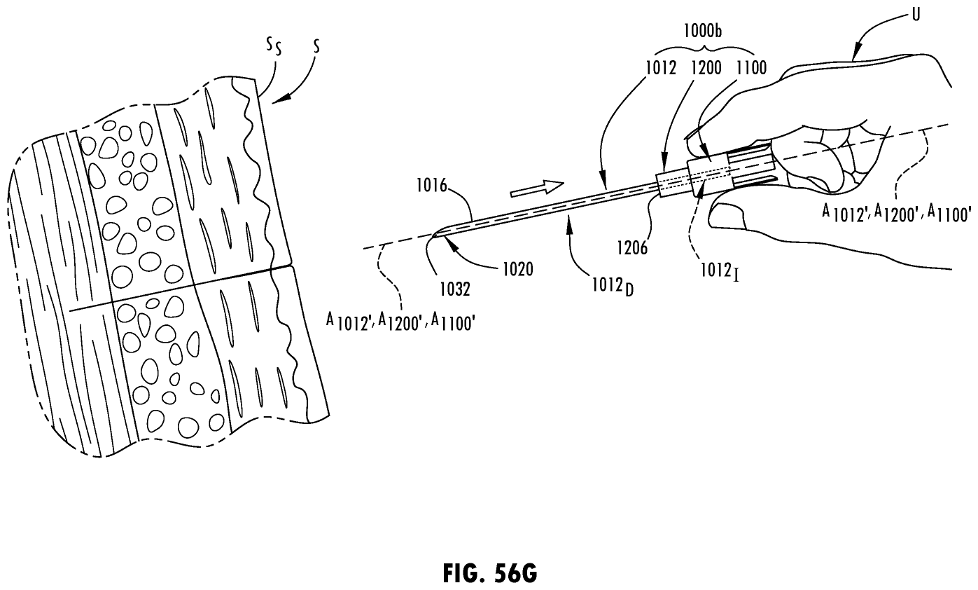

As seen at FIGS. 55 and 56A-56G, the cannula 1012 is configured to pierce an outer surface S_{S} (e.g., the skin or hide) of a subject S (e.g., animalia, such as a human or non-human). As seen at FIGS. 56C-556D, the purpose of piercing the skin or hide S_{S} of the animalia S may be directed to injecting a fluid F (e.g., a medicament, a pharmaceutical, a vaccine, an anesthetic, or the like) into the animalia S. In other examples, the purpose of piercing the skin or hide S_{S} of the animalia S may be directed to the purpose of drawing a fluid F (e.g., blood) from the animalia S. Accordingly, the cannula 1012 may be referred to as a hypodermic cannula, and, as such, the assembly 1000 may be referred to as a hypodermic interface assembly as a result of the cannula 1012 being capable of injecting or drawing a fluid F into/from the animalia S.

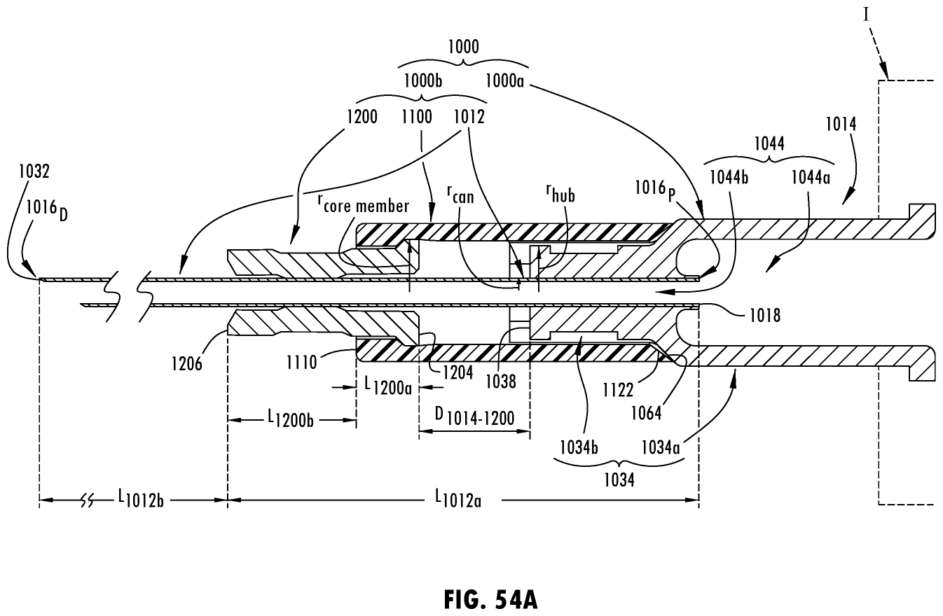

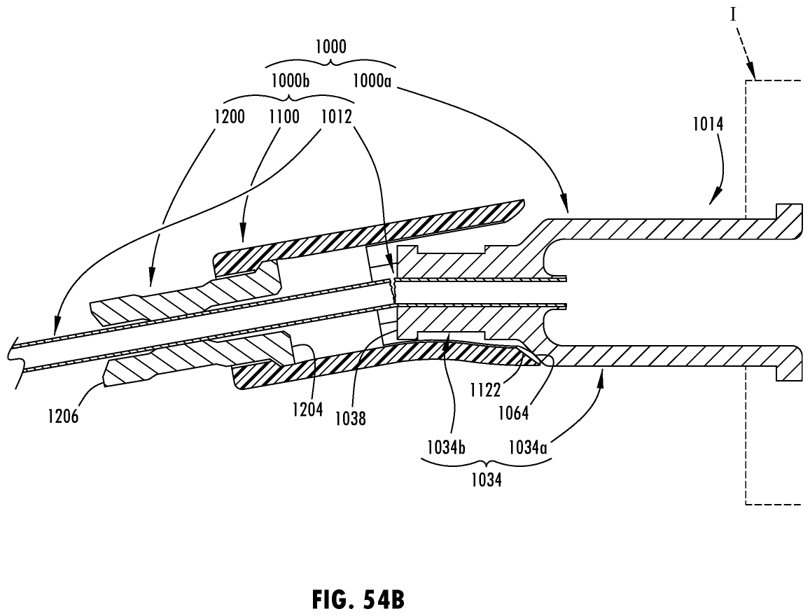

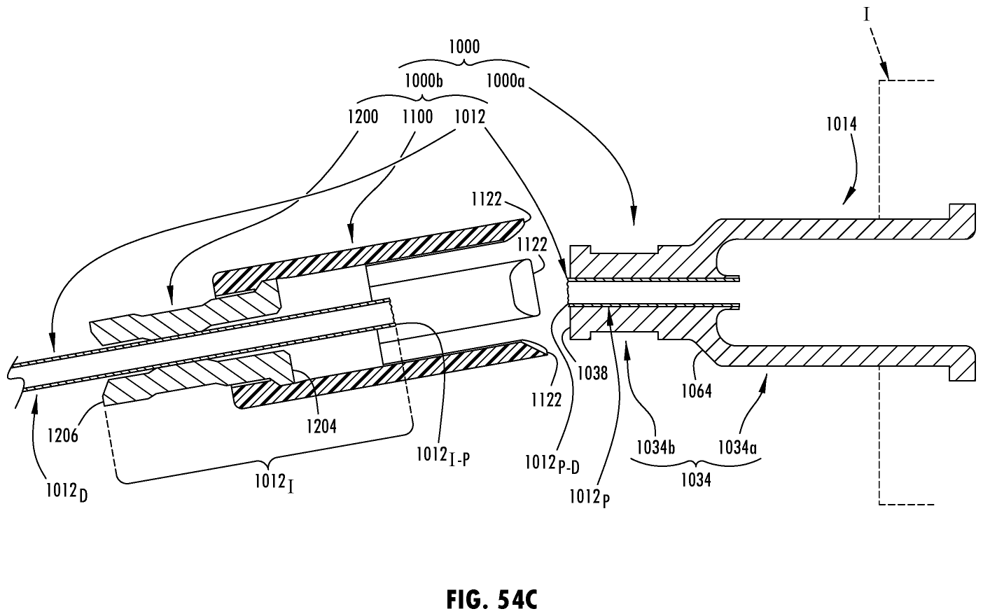

The design of the hypodermic interface assembly 1000 provides for: (1) a first portion (see, e.g., a first portion 1000a at FIGS. 54C and 56E) of the hypodermic interface assembly 1000 that is configured to remain attached to an injection gun I after the cannula 1012 is subjected to one or more radial forces X_{R} (see, e.g., FIG. 56D) relative to the central axis A₁₀₀₀-A₁₀₀₀ extending through the hypodermic interface assembly 1000; and (2) a second portion (see, e.g., a second portion 1000b at FIGS. 54C and 56E) of the hypodermic interface assembly 1000 that is configured to controllably (and/or predictably) separate from the first portion 1000a of the hypodermic interface assembly 1000 after the cannula 1012 is subjected to the one or more radial forces X_{R} relative to the central axis A₁₀₀₀-A₁₀₀₀ extending through the hypodermic interface assembly 1000. In some instances, controlled separation of the second portion 1000b of the hypodermic interface assembly 1000 from the first portion 1000a of the hypodermic interface assembly 1000 may occur after the cannula 1012 pierces the subject S (see, e.g., FIGS. 55 and 56B-56D). The subject S may be, for example, animalia, such as a human or non-human (i.e., an animal, such as a pig or swine). In other examples, the subject S may be an inanimate object. The predicable and controlled separation of the second portion 1000b of the hypodermic interface assembly 1000 from the first portion 1000a of the hypodermic interface assembly 1000 mitigates separation of the cannula 1012, alone, from a non-separated, non-broken, or unitary configuration of the hub 1014, which may otherwise result in the cannula 1012 being broken-off from the injection gun I and subsequently being lost below the outer surface S_{S} (e.g., the skin or hide) and within the flesh of the animalia.

As seen at FIGS. 42-43 the cannula 1012 is defined by a tube-shaped body 1016 having a proximal end 1016_{P} (see, e.g., FIG. 52A) and a distal end 1016_{D}. (see, e.g., FIGS. 42-43). The cannula 1012 is defined by a length extending between the proximal end 1016_{P} of the tube-shaped body 1016 and the distal end 1016_{D} of the tube-shaped body 1016.

The cannula 1012 may be formed using any desirable manufacturing procedure such as, for example: a drawing procedure, a molding procedure; a casting procedure; a machining procedure; a lathing procedure; or a combination thereof. The cannula 1012 is made from any desirable material such as, for example: a metallic material; a plastic material; or a combination thereof. In some examples, the cannula 1012 may be made from a stainless steel material. In other instances, the cannula 1012 may be made from an aluminum material. In yet other examples, the cannula 1012 may be made from a detectable material such as, for example, a detectable alloy, a ferromagnetic alloy, a magnetically-detectable material, a magnetic resonance imaging (MRI) detectable material, a material that absorbs X-rays, or the like.

The cannula 1012 may be defined in terms of ‘gauge size’ that takes into consideration skid/hide thickness of the subject S and/or a depth of injection of the subject S. The gauge size of the cannula 1012 may be defined in a series of industry standard numbers in which, for example, the lower the number, the wider the diameter of the cannula. Furthermore, the series of industry standard numbers defining gauge size of the cannula 1012 may be defined in a manner such that, for example, a higher gauge number indicates a smaller width of the cannula 1012. In some instances, the industry standard gauge sizes of the cannula 1012 may range from, for example: 14-Gauge; 16-Gauge; 18-Gauge; and 20-Gauge. Accordingly, in the range of exemplary industry standard numbers described above, a 14-Gauge cannula may be said to have a relatively largest diameter and highest strength (in terms of bendability/flexibility to a point where the cannula 1012 could potentially break/fail) whereas a 20-Gauge cannula may be said to have a relatively smallest diameter and lowest strength (in terms of bendability/flexibility to a point where the cannula 1012 could potentially break/fail).

A central axis A₁₀₁₂-A₁₀₁₂ (see, e.g., FIG. 52A) extends through an axial center of the tube-shaped body 1016 and along the length of the tube-shaped body 1016. As will be described in the following disclosure and shown at FIGS. 56D-56G, a portion of the length of the tube-shaped body 1016 may bend, flex, or deviate from the central axis A₁₀₁₂-A₁₀₁₂ that extends through an axial center of the tube-shaped body 1016, and, as a result, defines a “breakaway axis” of a distal broken portion of the length of the tube-shaped body 1016, which is seen at A₁₀₁₂′-A₁₀₁₂′. The axis A₁₀₁₂′-A₁₀₁₂′ may be said to be not aligned with and deviate away from the central axis A₁₀₀₀-A₁₀₀₀ extending through the hypodermic interface assembly 1000 when the cannula 1012 is arranged as a component of the hypodermic interface assembly 1000.

The tube-shaped body 1016 is further defined by a proximal end surface 1018 (see, e.g., FIG. 52A) at the proximal end 1016_{P} of the tube-shaped body 1016 and a distal end surface 1020 (see, e.g., FIGS. 42-43) at the distal end 1016D (see, e.g., FIGS. 42-43) of the tube-shaped body 1016. The tube-shaped body 1016 is further defined by an outer surface 1022 extending between the proximal end surface 1018 and the distal end surface 1020. With reference to FIG. 52A, the tube-shaped body 1016 is further defined by an inner surface 1024 extending between the proximal end surface 1018 and the distal end surface 1020. The inner surface 1024 further defines a passage 1026 (see, e.g., FIG. 52A) extending through the tube-shaped body 1016. The proximal end surface 1018 defines a proximal opening 1028 that is in fluid communication with the passage 1026. The distal end surface 1020 defines a distal opening 1030 (see, e.g., FIGS. 42-43) that is in fluid communication with the passage 1026.

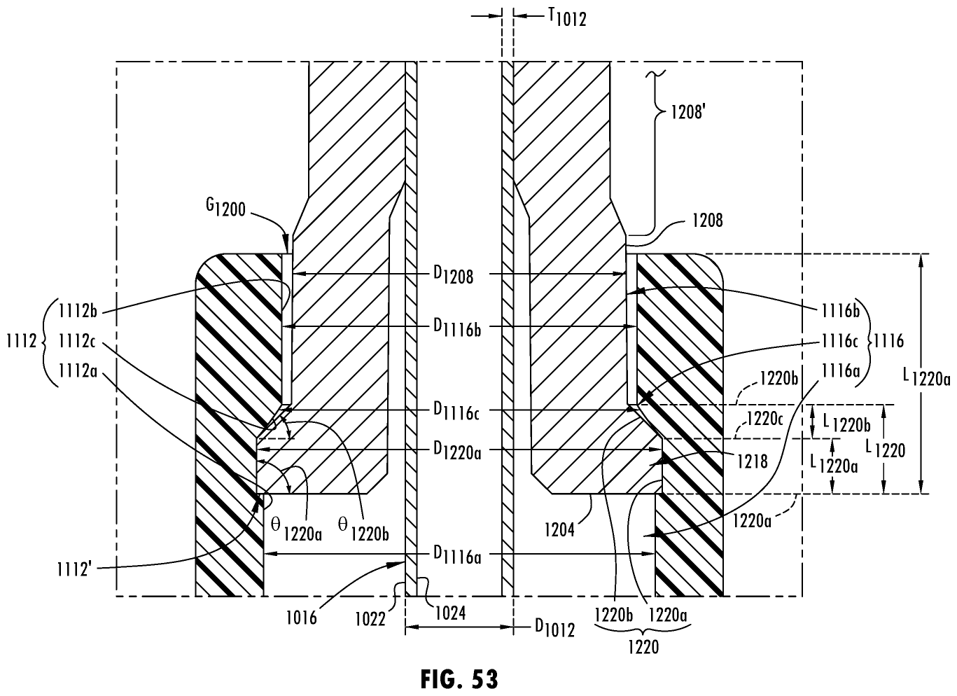

With reference to FIG. 53 (which illustrates an enlarged cross-section view of a portion of the hypodermic interface assembly 1000), the body 1016 of the cannula 1012 is defined by a thickness T₁₀₁₂ extending between the outer surface 1022 of the body 1016 and the inner surface 1024 of the body 1016. The outer surface 1022 further defines an outer diameter D₁₀₁₂ of the cannula 1012 that is referenced from the central axis A₁₀₁₂-A₁₀₁₂, which may be coincident with respective central axes A₁₀₀₀-A₁₀₀₀ and A₁₀₁₄-A₁₀₁₄ (see, e.g., FIG. 52A) of each of the hypodermic interface assembly 1000 and the hub 1014. The inner surface 1024 further defines the passage 1026 to have a passage diameter. The passage 1026 is in fluid communication with the proximal opening 1028 and the distal opening 1030 in order to permit: (1) passage of a fluid F (see, e.g., FIGS. 56C-56D) into the tube-shaped body 1016 at the proximal opening 1028; (2) through the passage 1026 in a direction from the proximal end 1016_{P} of the tube-shaped body 1016 and towards the distal end 1016D of the tube-shaped body 1016; and (3) out of the distal opening 1030.

The proximal end surface 1018 extends from the outer surface 1022 substantially perpendicularly, and, as such, defines the proximal end surface 1018 to be blunted or non-sharpened. Furthermore, the proximal opening 1028 formed by the proximal end surface 1018 may define a substantially circular-shaped geometry that is defined by a proximal opening diameter that is substantially similar to the passage diameter of the passage 1026.

As seen at, for example, FIG. 43, the distal end surface 1020 extends from the outer surface 1022 at a beveled angle, and, as such, the distal end surface 1020 may be referred to as a beveled distal end surface that terminates at or defines a sharp piercing tip 1032. The beveled distal end surface 1020 may be defined by any desirable beveled angle that forms, for example, a “standard bevel,” a “short bevel,” or a “true short bevel.” Because the beveled distal end surface 1020 extends from the outer surface 1022 at a beveled angle, the distal opening 1030 may be defined by an oval-shaped geometry. In one embodiment, the distal end surface 1020 may be defined by three separate beveled cuts.

As seen at FIGS. 42-43, the hub 1014 is defined by a substantially tube-shaped body 1034 having a first portion 1034a and a second portion 1034b. Referring to FIG. 52A, the tube-shaped body 1034 includes a proximal end 1034_{P} and a distal end 1034_{D}. With continued reference to FIG. 52A, the hub 1014 is defined by a length L₁₀₁₄ extending between the proximal end 1034_{P} of the substantially tube-shaped body 1034 and the distal end 1034D of the substantially tube-shaped body 1034.

The hub 1014 may be formed using any desirable manufacturing procedure such as, for example: a molding procedure; a casting procedure; a machining procedure; a lathing procedure; or a combination thereof. The hub 1014 is made from any desirable material such as, for example: a metallic material; a plastic material; or a combination thereof. In some examples, the hub 1014 may be made from a stainless steel material. In other instances, the hub 1014 may be made from an aluminum material, brass, steel, or alloys. In other examples, the hub 1014 may be made from plastic materials including but not limited to polypropylene (PP), polyethylene terephthalate (PET), polyamides (e.g., nylon 6, nylon 6, 6, thermosetting plastics such as polyester resins, epoxy resins, acrylics), and the like. Furthermore, in some instances, the hub 1014 may be finished with an anodization, a polishing, an electro-polishing, a coating, a paint or the like with, for example, a highly visible finish (e.g., a dye, a fluorescent coating, a phosphorescent coating, a bright gloss, matt color finish, or the like that preferably is not similar to the flesh tone or color of the surface S_{S} of the flesh of the animalia).

With continued reference to FIG. 52A, the substantially tube-shaped body 1034 is further defined by a proximal end surface 1036 at the proximal end 1034_{P} of the substantially tube-shaped body 1034 and a distal end surface 1038 at the distal end 1034_{D} of the substantially tube-shaped body 1034. The substantially tube-shaped body 1034 is further defined by an outer surface 1040 extending between the proximal end surface 1036 and the distal end surface 1038. The substantially tube-shaped body 1034 is further defined by an inner surface 1042 extending between the proximal end surface 1036 and the distal end surface 1038.

The inner surface 1042 further defines a passage 1044 extending through the substantially tube-shaped body 1034. The proximal end surface 1036 defines a proximal opening 1046 that is in fluid communication with the passage 1044. The distal end surface 1038 defines a distal opening 1048 that is in fluid communication with the passage 1044.

As seen at FIGS. 42-43 and 52A, a ring portion 1050 projects radially outwardly away from a central axis A₁₀₁₄-A₁₀₁₄ (see, e.g., FIG. 52A) away from the outer surface 1040 of the substantially tube-shaped body 1034. The ring portion 1050 may be alternatively referred to as a barrel-engaging portion that is configured to be connected to a barrel portion I_{B} of an injection gun I (see, e.g., FIG. 55). Referring to FIG. 52A, the barrel-engaging portion 1050 is defined by an outer side surface 1052 that extends between the proximal end surface 1036 and a distal shoulder surface 1054. The barrel-engaging portion 1050 may be defined by a thickness extending between the proximal end surface 1036 and the distal shoulder surface 1054. The barrel-engaging portion 1050 may generally define a Luer lock.

As seen at FIG. 52A, the outer surface 1040 defining the first portion 1034a of the substantially tube-shaped body 1034 may define a substantially circular-shaped geometry that defines a first outer diameter of the hub 1014. The outer side surface 1052 of the barrel-engaging portion 1050 may define a substantially circular-shaped geometry that defines a second outer diameter of the hub 1014 that is greater than the first outer diameter of the hub 1014. The outer surface 1040 defining the second portion 1034b of the substantially tube-shaped body 1034 may further define another substantially circular-shaped geometry that further defines a third outer diameter of the hub 1014 that is less than the second outer diameter of the hub.

As seen at FIGS. 42-43, the substantially circular-shaped geometry of the outer side surface 1052 of the barrel-engaging portion 1050 may be interrupted by a first radially-outward projection or ear 1056 and a second radially-outward projection or ear 1058 that extend beyond the second outer diameter of the hub 1014. The first radially-outward projection or ear 1056 may be arranged opposite of or offset approximately 180° from the second radially-outward projection or ear 1058.

As seen at FIG. 52A, the inner surface 1042 of the substantially tube-shaped body 1034 includes a first inner surface portion 1042a, a second inner surface portion 1042b, and a third inner surface portion 1042c. Each of the first inner surface portion 1042a and the second inner surface portion 1042b generally circumscribe the central axis A₁₀₁₄-A₁₀₁₄ of the hub 1014. The third inner surface portion 1042c connects the first inner surface portion 1042a to the second inner surface portion 1042b; furthermore, the third inner surface portion 1042c may be substantially orthogonal to the central axis A₁₀₁₄-A₁₀₁₄ of the hub 1014. The third inner surface portion 1042c may be substantially perpendicular with respect to each of the first inner surface portion 1042a and the second inner surface portion 1042b; in some implementations, the transition of each of the first inner surface portion 1042a and the second inner surface portion 1042b to the third inner surface portion 1042c may be defined by a curved or arcuate segment. As will be seen in the following disclosure at FIGS. 52A-52B, after material deformation of at least a portion of, for example, the second portion 1034b of the substantially tube-shaped body 1034 of the hub 1014 (e.g., by crimping a portion of, for example, the second portion 1034b of the substantially tube-shaped body 1034 of the hub 1014 after the cannula 1012 is interfaced with the hub 1014 as seen as FIG. 52B), the curved or arcuate segment joining the second inner surface portion 1042b to the third inner surface portion 1042c may change in shape as a result of the material shifting or “flowing”, and, as such, a portion of the third inner surface portion 1042c that extends from the second inner surface portion 1042b may define a frustoconical surface portion surrounding the cannula 1012.

As seen at FIG. 52A, the first inner surface portion 1042a of the inner surface defines a first passage portion 1044a of the passage 1044. The second inner surface portion 1042b defines a second passage portion 1044b of the passage 1044.

The first passage portion 1044a defines a first passage diameter of the passage 1044. The second passage portion 1044b defines a second passage diameter of the passage 1044. The first passage diameter is greater than the second passage diameter. The second passage diameter is approximately equal to but slightly greater than the outer diameter D₁₀₁₂ (see, e.g., FIG. 53) of the cannula 1012.

The first passage portion 1044a of the passage 1044 is in fluid communication with the proximal opening 1046 of the hub 1014, and the second passage portion 1044b of the passage 1044 is in fluid communication with the distal opening 1048 of the hub 1014. Furthermore, the first passage portion 1044a is in fluid communication with the second passage portion 1044b by way of an intermediate opening 1047. Accordingly, the passage 1044 permits: (1) passage of a fluid F (see, e.g., FIGS. 56C-56D) into the substantially tube-shaped body 1034 at the proximal opening 1046; (2) through the first passage portion 1044a of the passage 1044 in a direction from the proximal end 1034_{P} of the substantially tube-shaped body 1034 and towards the intermediate opening 1047 defined by the third inner surface portion 1042c; (3) through the intermediate opening 1047 that defines a proximal opening of the second passage portion 1044b of the passage 1044; (4) through the second passage portion 1044b of the passage 1044 in a direction from the intermediate opening 1047 and towards the distal end 1034D of the substantially tube-shaped body 1034; and (5) out of the distal opening 1048.

The proximal opening 1046 formed by the proximal end surface 1036 may define a substantially circular-shaped geometry that is defined by a proximal opening diameter that is substantially similar to the first passage diameter of the first passage portion 1044a. The intermediate opening 1047 formed by the third inner surface portion 1042c of the inner surface 1042 of the substantially tube-shaped body 1034 may define a substantially circular-shaped geometry that is defined by an intermediate opening diameter that is substantially equal to the second passage diameter. The distal opening 1048 formed by the distal end surface 1038 may define a substantially circular-shaped geometry that is defined by a distal opening diameter that is substantially similar to the second passage diameter. Although some of the dimensions/diameters/geometries are descried above to be substantially similar or the same, the view of the hub 1014 in the Figures (e.g., at FIG. 52A) are exemplary and are not to scale. In some instances, the first passage portion 1044a may be formed to include a draft angle (e.g., a 1° draft angle) that, for example, may assist in the removal of the hub 1014 from tooling when the hub 1014 is formed. Accordingly, the first passage diameter of the first passage portion 1044a may progressively decrease in diameter as the first passage diameter of the first passage portion 1044a extends in a direction from the proximal end surface 1036 of the hub 1014 toward the distal end surface 1038 of the hub 1014.

Referring to FIGS. 42-43, one or more ribs 1060 may project radially outwardly away from a central axis A₁₀₁₄-A₁₀₁₄ away from an outer body surface portion 1062 defined by the outer surface 1040 of the substantially tube-shaped body 1034. The one or more ribs 1060 may include, for example, a first rib, a second rib, a third rib, and a fourth rib.

The one or more ribs 1060 may increase the structural integrity of the substantially tube-shaped body 1034 of the hub 1014. In some configurations, the one or more ribs 1060 may arise from mold relief features during the manufacturing process of the substantially tube-shaped body 1034 of the hub 1014. Furthermore, the one or more ribs 1060 may be configured to engage packaging (not shown). Engagement of the one or more ribs 1060 with the packaging may assist in containing the cannula 1012 and the hub 1014 during shipping and/or assist in engagement/disengagement of the hub 1014 with/from the injection gun I. As seen throughout the Figures, an outer surface portion of each rib of the one or more ribs may extend radially outwardly, defining a lug portion; the lug portion may, for example be defined by an inclined or beveled surface (not shown). Each lug portion may be sized for engagement with the packaging.

Each rib of the one or more ribs 1060 includes a distal end and a proximal end. The proximal end of each rib of the one or more ribs 1060 extends from the distal shoulder surface 1054 of the barrel-engaging portion 1050. The distal end of each rib of the one or more ribs 1060 extends in a direction toward the distal end surface 1038 of the substantially tube-shaped body 1034 of the hub 1014 and terminates at, before, or near an outer shoulder surface portion 1064 (see also FIGS. 52A-52H) defined by the outer surface 1040 of the substantially tube-shaped body 1034 of the hub 1014. Each rib of the one or more ribs 1060 may define a substantially rectangular body that terminates with a substantially triangular body portion defined by the distal end of each rib of the one or more ribs 1060.

The outer shoulder surface portion 1064 extends from a distal-most end of the outer body surface portion 1062 of the outer surface 1040 of the substantially tube-shaped body 1034 of the hub 1014. In some configurations, the outer shoulder surface portion 1064 may define a dome-shaped or curved outer shoulder surface portion.

A distal-most end of the outer shoulder surface portion 1064 terminates at an outer head surface portion 1068, which is generally defined by the second portion 1034b of the substantially tube-shaped body 1034 of the hub 1014. The outer head surface portion 1068 generally circumscribes the central axis A₁₀₁₄-A₁₀₁₄ of the hub 1014. The outer head surface portion 1068 of the outer surface 1040 of the substantially tube-shaped body 1034 defines the third outer diameter of the hub 1014.

Referring now to FIGS. 42-47, an exemplary configuration of the core member 1200 is described. The core member 1200 is defined by a substantially tube-shaped body 1202. The tube-shaped body 1202 includes a proximal end 1202_{P} (see, e.g., FIG. 46) and a distal end 1202_{D} (see, e.g., FIG. 47). Referring to FIG. 45, the core member 1200 is defined by a length L₁₂₀₀ extending between the proximal end 1202_{P} of the substantially tube-shaped body 1202 and the distal end 1202_{D} of the substantially tube-shaped body 1202.

The substantially tube-shaped body 1202 of the core member 1200 may be preformed, preshaped, or preconfigured using any desirable manufacturing procedure such as, for example: a molding procedure; a casting procedure; a machining procedure; a lathing procedure; or a combination thereof. The substantially tube-shaped body 1202 of the core member 1200 is made from any desirable material such as, for example: a metallic material; a plastic material; or a combination thereof. In some examples, the substantially tube-shaped body 1202 of the core member 1200 may be made from a stainless steel material. In other instances, the substantially tube-shaped body 1202 of the core member 1200 may be made from an aluminum material, brass, steel, or alloys. In other examples, the substantially tube-shaped body 1202 of the core member 1200 may be made from plastic materials including but not limited to polypropylene (PP), polyethylene terephthalate (PET), polyamides (e.g., nylon 6, nylon 6, 6, thermosetting plastics such as polyester resins, epoxy resins, acrylics), and the like. Furthermore, in some instances, the substantially tube-shaped body 1202 of the core member 1200 may be finished with an anodization, a polishing, an electro-polishing, a coating, a paint or the like with, for example, a highly visible finish (e.g., a dye, a fluorescent coating, a phosphorescent coating, a bright gloss, matt color finish, or the like that preferably is not similar to the flesh tone or color of the surface S_{S} of the flesh of the animalia).

With continued reference to FIG. 45, the substantially tube-shaped body 1202 is further defined by a proximal end surface 1204 at the proximal end 1202_{P} of the substantially tube-shaped body 1202. The substantially tube-shaped body 1202 is further defined by a distal end surface 1206 at the distal end 1202_{D} of the substantially tube-shaped body 1202. As will be described in the following disclosure, a distal portion L_{1200b} (see, e.g., FIGS. 45, 52H, and 54A) of the length L₁₂₀₀ of the substantially tube-shaped body 1202 of the core member 1200 is arranged beyond a distal end surface 1110 of the cannula carrier 1100 such that the distal end surface 1206 of the substantially tube-shaped body 1202 of the core member 1200, which is a distal-most portion of the substantially tube-shaped body 1202 of the core member 1200, is arranged distally beyond the distal end surface 1110 of the cannula carrier 1100. Accordingly, as seen at FIGS. 56B-56F, when the hypodermic interface assembly 1000 is interfaced with the outer surface S_{S} of the subject S, the distal end surface 1206 of the substantially tube-shaped body 1202 of the core member 1200 is disposed adjacent the outer surface S_{S} of the subject S, whereas the distal end surface 1110 of the cannula carrier 1100 does not engage and is spaced away the outer surface S_{S} of the subject S at a distance according to the distal portion L_{1200b} of the length L₁₂₀₀ of the substantially tube-shaped body 1202 of the core member 1200.

The substantially tube-shaped body 1202 is further defined by an outer surface 1208 extending between the proximal end surface 1204 and the distal end surface 1206. As seen at FIG. 44, the outer surface 1208 defines an outer diameter D₁₂₀₈ of the substantially tube-shaped body 1202. As also seen at FIG. 45, the substantially tube-shaped body 1202 is further defined by an inner surface 1210 extending between the proximal end surface 1204 and the distal end surface 1206.

The inner surface 1210 further defines a cannula-receiving-passage 1212 extending through the substantially tube-shaped body 1202. As seen at FIG. 45, the inner surface 1210 defines a passage diameter D₁₂₁₂ of the cannula-receiving-passage 1212 extending through the substantially tube-shaped body 1202. The proximal end surface 1204 defines a proximal opening 1214 that is in fluid communication with the cannula-receiving-passage 1212. The distal end surface 1206 defines a distal opening 1216 that is in fluid communication with the cannula-receiving-passage 1212.

With reference to FIG. 45, the passage diameter D₁₂₁₂ of the cannula-receiving-passage 1212 may be a constant, non-changing diameter along the length L₁₂₀₀ of the substantially tube-shaped body 1202. However, as will be explained in the following disclosure at FIGS. 52D-52E, with the cannula 1012 arranged within the cannula-receiving-passage 1212, a portion 1208′ (see also FIG. 53) of the outer side surface 1208 of the substantially tube-shaped body 1202 the core member 1200 may be deformed (e.g., by crimping) along an intermediate portion L₁₂₀₀′ of the length L₁₂₀₀ of the substantially tube-shaped body 1202. Accordingly, as seen at FIG. 52E, as a result of deforming the portion 1208′ of the outer side surface 1208 of the substantially tube-shaped body 1202, a portion 1210′ of the inner surface 1210 (that also extends along the intermediate portion L₁₂₀₀′ of the length L₁₂₀₀ of the substantially tube-shaped body 1202) defining a portion 1212′ of the cannula-receiving-passage 1212 is also deformed.

Deformation of the portion 1210′ of the inner surface 1210 of the substantially tube-shaped body 1202 results in a portion D₁₂₁₂′ (see, e.g., FIG. 52E) of the passage diameter D₁₂₁₂ extending along the intermediate portion L₁₂₀₀′ of the length L₁₂₀₀ of the substantially tube-shaped body 1202 also being deformed. As a result, the portion 1210′ of the inner surface 1210 of the substantially tube-shaped body 1202 of the core member 1200 is joined to a portion 1022′ of the outer surface 1022 of the body 1016 of the cannula 1012 such that, as seen at FIG. 52E, a portion of the core member 1200 is said to be directly joined to a portion of the cannula 1012 (prior to arranging the cannula carrier 1100 about the cannula 1012).

After deforming the core member 1200 as described above, the cannula-receiving-passage 1212 allows for passage of a fluid F (e.g., within cannula 1012 arranged within the substantially tube-shaped body 1202; FIG. 56C) into the proximal opening 1214; through the cannula-receiving-passage 1212 in a direction from the proximal end 1202_{P} of the substantially tube-shaped body 1202; and out of the distal opening 1216.

The proximal opening 1214 formed by the proximal end surface 1204 may define a substantially circular-shaped geometry that is defined by a proximal opening diameter that is substantially similar to the passage diameter D₁₂₁₂ of the cannula-receiving-passage 1212. The distal opening 1216 formed by the distal end surface 1206 may define a substantially circular-shaped geometry that is defined by a distal opening diameter that is substantially similar to the passage diameter D₁₂₁₂ of the cannula-receiving-passage 1212.

As seen at FIGS. 44-45, the core member 1200 includes a ring portion 1218. The ring portion 1218 projects radially outwardly away from a central axis A₁₂₀₀-A₁₂₀₀ (see, e.g., FIG. 45) away from the outer surface 1208 of the substantially tube-shaped body 1202. The ring portion 1218 may be alternatively referred to as a cannula-carrier-engaging portion that is configured to be connected to or arranged adjacent at least a portion of the cannula carrier 1100 (see, e.g., FIGS. 52F-52H and 53).

The cannula-carrier-engaging portion 1218 is defined by an outer side surface 1220 having a first outer side surface portion 1220a and a second outer side surface portion 1220b. The first outer side surface portion 1220a is defined by a first outer side surface portion length L₁₂₂₀ₐ (as seen at, e.g., FIG. 53). The second outer side surface portion 1220b is defined by a second outer side surface portion length L_{1220b} (as seen at, e.g., FIG. 53). Collectively, the first outer side surface portion length L₁₂₂₀ₐ and the second outer side surface portion length L_{1220b} define a length L₁₂₂₀ (as seen at, e.g., FIG. 53) of the outer side surface 1220.

Referring to FIG. 53, the outer side surface 1220 of the cannula-carrier-engaging portion 1218 is defined by a proximal end 1222a, a distal end 1222b and an intermediate region 1222c. The length L₁₂₂₀ₐ of the first outer side surface portion 1220a extends between the proximal end 1222a of the outer side surface 1220 of the cannula-carrier-engaging portion 1218 and the intermediate region 1222c of the outer side surface 1220 of the cannula-carrier-engaging portion 1218. The length L₁₂₂₀ₐ of the first outer side surface portion 1220a of the outer side surface 1220 of the cannula-carrier-engaging portion 1218 extends from the proximal end surface 1204 of the substantially tube-shaped body 1202 of the core member 1200 at a first angle θ₁₂₂₀ₐ. The first angle θ₁₂₂₀ₐ may be approximately equal to 90°.

As also seen at FIG. 53, the length L_{1220b} of the second outer side surface portion 1220b extends between the intermediate region 1222c of the outer side surface 1220 of the cannula-carrier-engaging portion 1218 and the distal end 1222b of the outer side surface 1220 of the cannula-carrier-engaging portion 1218. The length L_{1220b} of the second outer side surface portion 1220b of the outer side surface 1220 of the cannula-carrier-engaging portion 1218 extends from the first outer side surface portion 1220a at a second angle θ_{1220b} that is different from the first angle θ₁₂₂₀ₐ. The second angle θ_{1220b} may be approximately equal to 45°.

With reference to FIG. 44, because first outer side surface portion 1220a of the outer side surface 1220 of the cannula-carrier-engaging portion 1218 extends from the proximal end surface 1204 of the substantially tube-shaped body 1202 at the first angle θ₁₂₂₀ₐ, which may be, in some configurations, approximately equal to 90°, the first outer side surface portion 1220a may define a substantially constant diameter D₁₂₂₀ₐ of the outer side surface 1220 of the cannula-carrier-engaging portion 1218. Because the second outer side surface portion 1220b of the outer side surface 1220 of the cannula-carrier-engaging portion 1218 extends from the first outer side surface portion 1220a of the outer side surface 1220 of the cannula-carrier-engaging portion 1218 at the second angle θ_{1220b}, which may be, in some configurations, approximately equal to 45°, the second outer side surface portion 1220b of the outer side surface 1220 of the cannula-carrier-engaging portion 1218 defines a non-constant, progressively-decreasing diameter as the second outer side surface portion 1220b of the outer side surface 1220 of the cannula-carrier-engaging portion 1218 distally extends from the intermediate region 1222c of the outer side surface 1220 of the cannula-carrier-engaging portion 1218 to the distal end 1222b of the outer side surface 1220 of the cannula-carrier-engaging portion 1218.

As seen at FIG. 53, the second outer side surface portion 1220b of the outer side surface 1220 of the cannula-carrier-engaging portion 1218 extends between and connects a proximal end of the outer side surface 1208 of the substantially tube-shaped body 1202 to a distal end of the first outer side surface portion 1220a of the outer side surface 1220 of the cannula-carrier-engaging portion 1218. Because the second outer side surface portion 1220b of the outer side surface 1220 of the cannula-carrier-engaging portion 1218 may be defined by the non-constant, changing diameter that decreases as the second outer side surface portion 1220b distally extends from the intermediate region 1222c of the outer side surface 1220 of the cannula-carrier-engaging portion 1218 toward the distal end 1222b of the outer side surface 1220 of the cannula-carrier-engaging portion 1218, the second outer side surface portion 1220b of the outer side surface 1220 of the cannula-carrier-engaging portion 1218 may define or be alternatively referred to as a shoulder surface portion of the outer side surface 1220 of the cannula-carrier-engaging portion 1218.

Referring to FIGS. 48-51, an exemplary cannula carrier 1100 is shown. The cannula carrier 1100 may be formed using any desirable manufacturing procedure such as, for example: a molding procedure; a casting procedure; a machining procedure; or a combination thereof. The cannula carrier 1100 may be made from any desirable material such as, for example: a metallic material; a plastic material; or a combination thereof. In some examples, the cannula carrier 1100 may be made with a high visibility dye or pigment such as, for example, a brightly colored pigment, a fluorescent pigment, a phosphorescent pigment, retroreflective partially mirrored glass beads, metallic flake pigment, or the like that preferably is not similar to the flesh tone or color of the surface S_{S} of the flesh of the animalia. Furthermore, when the cannula carrier 1100 is formed, the cannula carrier 1100 may include an over-molded RFID component (not shown) embedded in the material or an RFID sticker (not shown) or other identifying information disposed upon one or more of the inner surface 1112 and the outer surface 1114 of the cannula carrier 1100 in order to determine the location and/or serial number or other identifier associated with the cannula carrier 1100.

As seen at FIGS. 48-49, the cannula carrier 1100 includes a head portion 1102 and a plurality of leg portions 1104 defined by, for example, four leg portions including a first leg portion 1104a, a second leg portion 1104b, a third leg portion 1104c, and a fourth leg portion 1104d. The head portion 1102 includes a body 1106 extending between a proximal end surface 1108 and a distal end surface 1110.

The body 1106 of the head portion 1102 also includes an inner surface 1112 (see, e.g., FIG. 49) and an outer surface 1114. With reference to FIG. 49, the inner surface 1112 of the body 1106 of the head portion 1102 includes a first inner surface portion 1112a, a second inner surface portion 1112b (see also, e.g., FIG. 48), and an intermediate inner surface portion 1112c extending between and connecting the first inner surface portion 1112a to the second inner surface portion 1112b. The body 1106 of the head portion 1106 is also defined by a thickness T₁₁₀₆ (see, e.g., FIG. 49) extending between the inner surface 1112 and the outer surface 1114.

With reference to FIG. 49, the inner surface 1112 of the body 1106 of the head portion 1102 of the cannula carrier 1100 defines an axial passage 1116. The axial passage 1116 includes a first passage portion 1116a, a second passage portion 1116b, and an intermediate passage portion 1116c extending between and fluidly connecting the first passage portion 1116a to the second passage portion 1116b. The first passage portion 1116a is defined by the first inner surface portion 1112a of the inner surface 1112 of the body 1106 of the head portion 1102. The second passage portion 1116b is defined by the second inner surface portion 1112b of the inner surface 1112 of the body 1106 of the head portion 1102. The intermediate passage portion 1116c is defined by the intermediate inner surface portion 1112c of the inner surface 1112 of the body 1106 of the head portion 1102.

Access to the axial passage 1116 is permitted by a proximal opening 1118 (see, e.g., FIGS. 49 and 51) and a distal opening 1120 (see, e.g., FIGS. 48-51). As seen at FIG. 49, the first inner surface portion 1112a extends from the proximal end surface 1108 and defines the proximal opening 1118. The second inner surface portion 1112b extends from the distal end surface 1110 and defines the distal opening 1120.

With reference to FIG. 49, the first passage portion 1116a is defined by a first passage diameter D₁₁₁₆ₐ. In some configurations, the first passage diameter D₁₁₁₆ₐ may be defined by a substantially constant, non-changing diameter as the first inner surface portion 1112a distally extends along a length L₁₁₀₆ of the body 1106 of the head portion 1102 from the proximal end surface 1108 of the body 1106 of the head portion 1102 toward the distal end surface 1110 of the body 1106 of the head portion 1102.

With continued reference to FIG. 49, the second passage portion 1116b is defined by a second passage diameter D_{1116b}. In some configurations, the second passage diameter D_{1116b} may be defined by a substantially constant, non-changing diameter as the second inner surface portion 1112b distally extends along the length L1106 of the body 1106 of the head portion 1102 from the proximal end surface 1108 of the body 1106 of the head portion 1102 toward the distal end surface 1110 of the body 1106 of the head portion 1102.

As also seen at FIG. 49, the intermediate passage portion 1116c is defined by a third passage diameter D_{1116c}. In some configurations, the third passage diameter D_{1116c} may be defined by a substantially non-constant, changing diameter as the intermediate inner surface portion 1112c distally extends along the length L₁₁₀₆ of the body 1106 of the head portion 1102 from the proximal end surface 1108 of the body 1106 of the head portion 1102 toward the distal end surface 1110 of the body 1106 of the head portion 1102.

In some configurations, the first passage diameter D₁₁₁₆ₐ is greater than the second passage diameter D_{1116b}. In some implementations, the third passage diameter D_{1116c} progressively decreases in diameter as the third passage diameter D₁₁₁₆c distally extends along the length L₁₁₀₆ of the body 1106 of the head portion 1102 from the proximal end surface 1108 of the body 1106 of the head portion 1102 toward the distal end surface 1110 of the body 1106 of the head portion 1102.

With reference to FIG. 53, the second passage diameter D_{1116b} is approximately equal to but slightly greater than the outer diameter D₁₂₀₈ defined by the outer surface 1208 of the substantially tube-shaped body 1202 of the core member 1200. Accordingly, in some implementations, a spacing or gap G₁₂₀₀ may be formed between the proximal end of the outer surface 1208 of the substantially tube-shaped body 1202 of the core member 1200 and the second inner surface portion 1112b of the inner surface 1112 of the body 1106 of the head portion 1102 of the cannula carrier 1100 when the cannula carrier 1100 is secured to the core member 1200. In some implementations, the outer diameter D₁₂₀₈ defined by the outer surface 1208 of the substantially tube-shaped body 1202 may range from, for example: about 1.4 mm to about 5.0 mm; about 1.45 mm to about 5.25 mm; about 1.50 mm to about 5.30 mm; about 1.60 mm to about 5.40 mm; about 1.90 mm to about 4.50 mm; about 1.95 mm to about 4.75 mm; about 2.00 mm to about 4.80 mm; about 2.10 mm to about 4.90 mm; about 2.90 mm to about 3.50 mm; about 2.95 mm to about 3.75 mm; about 3.00 mm to about 3.80 mm; or about 3.10 mm to about 3.90 mm. Furthermore, in some implementations, the second passage diameter D_{1116b} may range from, for example: about 1.60 mm to about 5.20 mm; about 1.65 mm to about 5.45 mm; about 1.70 mm to about 5.50 mm; about 1.80 mm to about 5.60 mm; about 2.10 mm to about 4.70 mm; about 2.15 mm to about 4.95 mm; about 2.20 mm to about 5.00 mm; about 2.30 mm to about 5.10 mm; about 3.10 mm to about 3.70 mm; about 3.15 mm to about 3.95 mm; about 3.20 mm to about 4.00 mm; or about 3.30 mm to about 4.10 mm.

As also seen at FIG. 53, the first passage diameter D₁₁₁₆ₐ is approximately equal to but slightly less than the substantially constant diameter D₁₂₂₀ₐ of the first outer side surface portion 1220a of the outer side surface 1220 of the cannula-carrier-engaging portion 1218 of the substantially tube-shaped body 1202 of the core member 1200. Accordingly, as will be described in the following disclosure, one or both of the first outer side surface portion 1220a and the second outer side surface portion 1220b of the outer side surface 1220 of the cannula-carrier-engaging portion 1218 of the core member 1200 may deform one or more portions of the inner surface 1112 of the body 1106 of the head portion 1106 of the head portion 1102 of the cannula carrier 1100 such that the cannula-carrier-engaging portion 1218 of the core member 1200 may be friction-fit-coupled to the inner surface 1112 of the body 1106 of the head portion 1102 of the cannula carrier 1100 and may “plug” or “fluidly seal” the distal opening 1120 of the cannula carrier 1100. In some implementations, the substantially constant diameter Duna of the first outer side surface portion 1220a of the outer side surface 1220 of the cannula-carrier-engaging portion 1218 of the substantially tube-shaped body 1202 of the core member 1200 may range from, for example: about 2.40 mm to about 6.00 mm; about 2.45 mm to about 6.35 mm; about 2.60 mm to about 6.40 mm; about 2.70 mm to about 6.50 mm; about 2.90 mm to about 5.50 mm; about 2.95 mm to about 5.75 mm; about 3.10 mm to about 5.90 mm; about 3.20 mm to about 6.00 mm; about 3.90 mm to about 4.50 mm; about 3.95 mm to about 4.75 mm; about 4.10 mm to about 4.90 mm; or about 4.20 mm to about 5.00 mm. Furthermore, in some implementations, the first passage diameter D₁₁₁₆ₐ may range from, for example: about 2.20 mm to about 5.80 mm; about 2.25 mm to about 6.95 mm; about 2.40 mm to about 6.20 mm; about 2.50 mm to about 6.30 mm; about 2.70 mm to about 5.30 mm; about 2.75 mm to about 5.45 mm; about 2.90 mm to about 5.70 mm; about 3.00 mm to about 5.80 mm; about 3.70 mm to about 4.30 mm; about 3.75 mm to about 4.45 mm; about 3.90 mm to about 4.70 mm; or about 4.00 mm to about 4.80 mm.

As will be described in the following disclosure at FIGS. 52F-52H, the cannula carrier 1100 is non-removably coupled or secured to the core member 1200 such that a proximal portion L₁₂₀₀ₐ (see, e.g., FIGS. 45, 52H, 53, and 54A) of the length L₁₂₀₀ of the substantially tube-shaped body 1202 of the core member 1200 is arranged within the axial passage 1116 extending through the body 1106 of the head portion 1102 of the cannula carrier 1100. Accordingly, in some configurations, with reference to FIG. 53, the cannula-carrier-engaging portion 1218, which is inclusive to the proximal portion L₁₂₀₀ₐ of the length L₁₂₀₀ of the substantially tube-shaped body 1202 of the core member 1200, may be arranged: (1) within one or both of the first passage portion 1116a and the intermediate passage portion 1116c of the head portion 1102 of the cannula carrier 1100; and (2) not within any of the second passage portion 1116b of the head portion 1102 of the cannula carrier 1100. In other configurations, the cannula-carrier-engaging portion 1218 of the core member 1200 may be arranged: (1) within one or both of a distal portion of the first passage portion 1116a and a proximal portion of the intermediate passage portion 1116c of the head portion 1102 of the cannula carrier 1100; and (2) not within any of the second passage portion 1116b of the head portion 1102 of the cannula carrier 1100. In yet other configurations, the cannula-carrier-engaging portion 1218 of the core member 1200 may be arranged within: (1) a distal portion of the first passage portion 1116a; (2) all of the intermediate passage portion 1116c of the head portion 1102 of the cannula carrier 1100; and (3) a proximal portion of the second passage portion 1116b of the head portion 1102 of the cannula carrier 1100.

Furthermore, as will be described in the following disclosure, the length L₁₂₀₀ of the substantially tube-shaped body 1202 is also configured to include a distal portion L_{1200b} (see, e.g., FIGS. 45, 52H, and 54A) of the length L₁₂₀₀ of the substantially tube-shaped body 1202 of the core member 1200. The distal portion L_{1200b} of the length L₁₂₀₀ of the substantially tube-shaped body 1202 of the core member 1200 is configured for arrangement outside of the axial passage 1116 extending through the body 1106 of the head portion 1102 of the cannula carrier 1100 such that the distal portion L_{1200b} of the length L₁₂₀₀ of the substantially tube-shaped body 1202 of the core member 1200 is arranged distally beyond the distal end surface 1110 of the body 1106 of the head portion 1102 of the cannula carrier 1100. With reference to FIG. 54A, in some implementations, the distal portion L_{1200b} of the length L₁₂₀₀ of the substantially tube-shaped body 1202 may range from, for example, about 0.00 mm to about 4.00 mm. However, in some implementations, the substantially tube-shaped body 1202 of the core member 1200 may not be configured for arrangement outside of the axial passage 1116; accordingly, in such instances, the distal portion L_{1200b} of the length L₁₂₀₀ of the substantially tube-shaped body 1202 may range from, for example: about −2.00 mm to about 4.00 mm; about −1.00 mm to about 2.00 m; about −0.50 mm to about 1.00; or about −0.10 mm to about 0.50 mm.

Referring to FIGS. 48-49, each leg portion 1104a, 1104b, 1104c, 1104d proximally axially extends from the proximal end surface 1108 of the body 1106 of the head portion 1102 of the cannula carrier 1100. Collectively, the leg portions 1104a, 1104b, 1104c, 1104d circumscribe a central axis A₁₁₀₀-A₁₁₀₀ (see, e.g., FIG. 49) extending through an axial center of the cannula carrier 1100. Furthermore, each leg portion 1104a, 1104b, 1104c, 1104d may be circumferentially offset approximately 90° from an adjacent leg portion 1104a, 1104b, 1104c, 1104d, defining an axial gap between each adjacent leg portion 1104a, 1104b, 1104c, 1104d.

Furthermore, as seen at FIGS. 48, 49, and 51, each leg portion 1104a, 1104b, 1104c, 1104d is defined by a proximal end surface 1122. As seen at FIGS. 52F-52H, each proximal end surface 1122 may be configured to matingly-couple with or be matingly-disposed-adjacent the outer shoulder surface portion 1064 defined by the outer surface 1040 of the substantially tube-shaped body 1034 of the hub 1014.

Referring to FIGS. 54A-54C, a first portion of the cannula 1012 non-removably-secured to the substantially tube-shaped body 1034 of the hub 1014 may define a first portion 1000a of the hypodermic interface assembly 1000 that is configured to remain attached to the injection gun I (see, e.g., FIGS. FIGS. 56A-56D) after the cannula 1012 is subjected to one or more radial forces X_{R} (see, e.g., FIG. 56D) relative to the central axis A₁₀₀₀-A₁₀₀₀ extending through the hypodermic interface assembly 1000. As also seen at FIGS. 54A-54C, the cannula carrier 1100 is indirectly attached to a second portion of the cannula 1012 by way of the core member 1200 (that is non-removably-connected to the second portion of the cannula 1012) for defining a second portion 1000b of the hypodermic interface assembly 1000. Accordingly, in some configurations, the cannula carrier 1100 may be said to be disconnected from the hub 1014 (by way of, for example, separating each proximal end surface 1122 of each leg portion 1104a, 1104b, 1104c, 1104d from the outer shoulder surface portion 1064 defined by the outer surface 1040 of the substantially tube-shaped body 1034 of the hub 1014) after the cannula 1012 is subjected to the one or more radial forces X_{R} relative to the central axis A₁₀₀₀-A₁₀₀₀ extending through the hypodermic interface assembly 1000. Accordingly, as will be explained in the following disclosure, upon predictably separating the cannula carrier 1100 from the substantially tube-shaped body 1034 of the hub 1014, a user may easily locate and grasp (see, e.g., FIG. 56F) the cannula carrier 1100 (that is non-removably-connected to the cannula 1012 by way of the core member 1200) in order to remove second portion of the cannula 1012 (see, e.g., FIG. 56G) from the flesh of the animalia S such that the second portion of the cannula 1012 is not lost within the flesh of the animalia S (should the one or more radial forces X_{R} relative to the central axis A₁₀₀₀-A₁₀₀₀ extending through the hypodermic interface assembly 1000 be imparted to the cannula 1012 during the course of utilizing the hypodermic interface assembly 1000, which may otherwise undesirably result in some or all of the cannula 1012 being separated from the injection gun I).

Referring to FIGS. 52A-52H, a method for assembling the hypodermic interface assembly 1000 is described. The hypodermic interface assembly 1000 is formed in several steps by assembling a plurality of sub-assemblies. For example, as seen at FIGS. 52A-52B, a first sub-assembly is formed that includes the first portion of the cannula 1012 being non-removably-joined to the hub 1014. Then, as seen at FIGS. 52C-52E, a second sub-assembly is formed that includes the core member 1200 non-removably-joined to the second portion of the cannula 1012 of the first sub-assembly (that includes the cannula 1012 the hub 1014). Lastly, as seen at FIGS. 52F-52H, a third sub-assembly is formed that includes the cannula carrier 1100 non-removably-joined to core member 1200 (as seen at, e.g., FIG. 53) of the second sub-assembly (that includes the cannula 1012, the hub 1014, and the core member 1200); the third sub-assembly may be alternatively referred to as the hypodermic interface assembly 1000.

Referring to FIGS. 52A-52B, a method for assembling a first sub-assembly (defined by the cannula 1012 being non-removably-joined to the hub 1014) of the hypodermic interface assembly 1000 is described. Firstly, at FIG. 52A, the components (i.e., the cannula 1012 and the hub 1014 of the first sub-assembly are axially aligned about a central axis A₁₀₀₀-A₁₀₀₀ (see also FIGS. 42-43). The central axis A₁₀₀₀-A₁₀₀₀ corresponds to, for example, the central axes A₁₀₁₂-A₁₀₁₂, A₁₀₁₄-A₁₀₁₄ of each of the cannula 1012 and the hub 1014.

As will be described in the following disclosure, the cannula 1012 is mechanically joined to any portion of the hub 1014 as a result of, for example, material deformation of at least a portion of, for example, the outer head surface portion 1068 of the outer surface 1040 of the substantially tube-shaped body 1034 of the hub 1014 (e.g., by crimping a portion of, for example, outer head surface portion 1068 of the outer surface 1040 of the substantially tube-shaped body 1034 of the hub 1014 as seen as FIG. 52B). Although the first sub-assembly of the hypodermic interface assembly 1000 is formed by a mechanical connection, the cannula 1012 may alternatively or additionally be joined to any portion of the hub 1014, such as, for example, with an adhesive (not shown), such as, for example: an acrylic adhesive, a cyanoacrylate adhesive, a ultra-violet (UV) curable adhesive, or the like. In other configurations the hub 1014 may be attached to the cannula 1012 by over-molding a material defining the hub 1014 relative the cannula 1012 (e.g., when the hub 1014 is formed from a moldable material such as a plastic material).

As seen at FIG. 52A, a portion of the cannula 1012 including the proximal end surface 1018 at the proximal end 1016_{P} of the tube-shaped body 1016 is shown arranged near the distal opening 1048 (that is in fluid communication with the second passage portion 1044b of the passage 1044 of the hub 1014) formed by the distal end surface 1038 of the hub 1014. The central axis A₁₀₁₂-A₁₀₁₂ of the cannula 1012 is axially aligned with the central axis A₁₀₁₄-A₁₀₁₄ of the hub 1014. The central axes A₁₀₁₂-A₁₀₁₂ and A₁₀₁₄-A₁₀₁₄ of each of the cannula 1012 and the hub 1014 correspond to the central axis A₁₀₀₀-A₁₀₀₀ of the hypodermic interface assembly 1000.

The outer surface 1022 of the tube-shaped body 1016 of the cannula 1012 defines an outer diameter of the cannula 1012, and the second passage diameter defined by the second passage portion 1044b of the passage 1044 is approximately equal to but slightly greater than the outer diameter of the cannula 1012 so that at least a portion of the second passage portion 1044b of the passage 1044 is configured to receive the cannula 1012. Then, the proximal end 1016_{P} of the tube-shaped body 1016 of the cannula 1012 is inserted according to the direction of the arrow Y through the distal opening 1048 formed by the distal end surface 1038 of the hub 1014 and then disposed within at least a portion of the second passage portion 1044b of the passage 1044 of the hub 1014. In some configurations as seen at, for example, FIG. 52B, the cannula 1012 may be arranged relative the hub 1014 such that the proximal end 1016_{P} of the tube-shaped body 1016 of the cannula 1012 is located beyond the third inner surface portion 1042c of the inner surface 1042 of the substantially tube-shaped body 1034. As such, a portion of the cannula 1012 is arranged within and entirely occupies the second passage portion 1044b of the passage 1044 of the hub 1014 while also being partially disposed within the first passage portion 1044a of the passage 1044 of the hub 1014. Furthermore, with reference to FIGS. 42-43 and 52B, a remainder of the length of the outer surface 1022 of the tube-shaped body 1016 of the cannula 1012 extends beyond the distal end surface 1038 of the hub 1014 and is not contained within the passage 1044 of the hub 1014.

Thereafter, as seen at FIG. 52B, the cannula 1012 is arranged within the passage 1044 of the hub 1014 in order to subsequently, for example, mechanically join the cannula 1012 to the hub 1014 by, for example, arranging the head surface portion 1068 of the outer surface 1040 of the substantially tube-shaped body 1034 of the hub 1014 within, for example, a crimping tool T. The crimping tool T may punch, crimp, swage or materially deform, for example, all or a portion of the head surface portion 1068 of the outer surface 1040 of the substantially tube-shaped body 1034 of the hub 1014 in order to mechanically connect all or a portion of the second inner surface portion 1042b of the inner surface 1042 of the substantially tube-shaped body 1034 of the hub 1014 to a portion of the length of the outer surface 1022 of the tube-shaped body 1016 of the cannula 1012 in a friction-fit relationship, an interference-fit relationship, or a mechanically-coupled relationship.

Accordingly, as seen at FIG. 52B, the cannula 1012 may be mechanically joined to the hub 1014 as a result of the material deformation of the portion of the hub 1014 by the crimping tool T. In some configurations as seen at FIG. 52B, the crimping tool T may punch, crimp, swage or materially deform, for example, a portion of the outer surface 1040 of the substantially tube-shaped body 1034. In some implementations, for example, the crimping tool T may punch, crimp, swage or materially deform (see, e.g., reference numeral 1068′), for example, a portion or all of the outer head surface portion 1068 of the outer surface 1040 of the substantially tube-shaped body 1034. Accordingly, in such implementations, the outer head surface portion 1068 of the outer surface 1040 of the substantially tube-shaped body 1034 may define crimping pockets (see, e.g., reference numeral 1068′) formed by the material deformation of the outer head surface portion 1068 of the outer surface 1040 of the substantially tube-shaped body 1034. The material deformation of the portion of the hub 1014 distinguishes a “deformed” hub 1014 that is mechanically connected to the cannula 1012 from a virgin or “non-deformed” hub 1014 (see, e.g., FIG. 52A) having an outer head surface portion 1068 of the outer surface 1040 of the substantially tube-shaped body 1034 that is not defined by crimping pockets 1068′.

Referring to FIGS. 52C-52E, a method for assembling a second sub-assembly (defined by the core member 1200 being non-removably-joined to the cannula 1012 of the first sub-assembly) of the hypodermic interface assembly 1000 is described. Firstly, at FIG. 52C, the components (i.e., the cannula 1012, the hub 1014, and the core member 1200) of the second sub-assembly are axially aligned about a central axis A₁₀₀₀-A₁₀₀₀ (see also FIGS. 42-43). The central axis A₁₀₀₀-A₁₀₀₀ corresponds to, for example, the central axes A₁₀₁₂-A₁₀₁₂, A₁₀₁₄-A₁₀₁₄, A₁₂₀₀-A₁₂₀₀ of each of the cannula 1012, the hub 1014, and the core member 1200.

As will be described in the following disclosure, the core member 1200 is mechanically joined to any portion of the cannula 1012 as a result of, for example, material deformation of at least a portion of, for example, the outer surface 1208 of the substantially tube-shaped body 1202 of the core member 1200 (e.g., by crimping a portion of, for example, the outer surface 1208 of the substantially tube-shaped body 1202 of the core member 1200 as seen as FIGS. 52D-52E). Although the second sub-assembly of the hypodermic interface assembly 1000 is formed by a mechanical connection, the cannula 1012 may alternatively or additionally be joined to any portion of the core member 1200, such as, for example, with an adhesive (not shown), such as, for example: an acrylic adhesive, a cyanoacrylate adhesive, a ultra-violet (UV) curable adhesive, or the like. In other configurations the core member 1200 may be attached to the cannula 1012 by over-molding a material defining the core member 1200 relative the cannula 1012 (e.g., when the core member 1200 is formed from a moldable material such as a plastic material).

As seen at FIG. 52C, the cannula-receiving-passage 1212 of the core member 1200 is co-axially aligned with the tube-shaped body 1016 of the cannula 1012 such that the central axis A₁₀₁₂-A₁₀₁₂ of the cannula 1012 is axially aligned with the central axis A₁₂₀₀-A₁₂₀₀ of the core member 1200. Furthermore, the central axes A₁₀₁₂-A₁₀₁₂, A₁₀₁₄-A₁₀₁₄, A₁₂₀₀-A₁₂₀₀ of each of the cannula 1012, the hub 1014, and the core member 1200 correspond to the central axis A₁₀₀₀-A₁₀₀₀ of the hypodermic interface assembly 1000. Subsequently, the distal end 1016_{D} of the tube-shaped body 1016 of the cannula 1012 is inserted through the proximal opening 1214 formed by the proximal end surface 1204 of the core member 1200. Thereafter, as seen at FIG. 52, the core member 1200 is positioned relative the cannula 1012 such that the distal end 1016D of the tube-shaped body 1016 of the cannula 1012 passes through the distal opening 1216 formed by the distal end surface 1206 of the core member 1200. Accordingly, the cannula 1012 is arranged relative the hub 1014 and the core member 1200 such that: (1) a first portion of the length of the cannula 1012 is arranged within the passage 1044 of the hub 1014; (2) a second portion of the length of the cannula 1012 is not arranged within the passages 1044, 1212 of the hub 1014 and the core member 1200 such that the second portion of the length of the cannula 1012 extends between the distal end surface 1038 of the hub 1014 and the proximal end surface 1204 of the core member 1200, defining a predetermined hub-core distance D₁₀₁₄₋₁₂₀₀; (3) a third portion of the length of the cannula 1012 is arranged within the cannula-receiving-passage 1212 of the core member 1200; and (4) a fourth portion of the length of the cannula 1012 is not arranged within the cannula-receiving-passage 1212 of the core member 1200 such that the fourth portion of the length of the cannula 1012 extends beyond the distal end surface 1206 of the core member 1200.

With reference to FIG. 52D, because the outer surface 1022 of the tube-shaped body 1016 of the cannula 1012 that defines an outer diameter of the cannula 1012 is approximately equal to but slightly less than the passage diameter D₁₂₁₂ of the cannula-receiving-passage 1212 of the core member 1200, the tube-shaped body 1016 of the cannula 1012 is configured to be arranged within at least a portion of the cannula-receiving-passage 1212 of the core member 1200. Arrangement of the cannula 1012 within the cannula-receiving-passage 1212 of the core member 1200 permits, for example, subsequent mechanical joining of the cannula 1012 to the core member 1200 by arranging the outer surface 1208 of the substantially tube-shaped body 1202 of the core member 1200 within, for example, a crimping tool T. The crimping tool T may punch, crimp, swage or materially deform, for example, all or a portion of the outer surface 1208 of the substantially tube-shaped body 1202 of the core member 1200 in order to mechanically connect all or a portion of the inner surface 1210 of the substantially tube-shaped body 1202 of the core member 1200 to a portion of the length of the outer surface 1022 of the tube-shaped body 1016 of the cannula 1012 in a friction-fit relationship, an interference-fit relationship, or a mechanically-coupled relationship.

Accordingly, as seen at FIG. 52E, the cannula 1012 may be mechanically joined to the core member 1200 as a result of the material deformation of the portion 1208′ of the outer side surface 1208 of the substantially tube-shaped body 1202 of the core member 1200 extending along the intermediate portion L₁₂₀₀′ of the length L₁₂₀₀ of the substantially tube-shaped body 1202 of the core member 1200 by the crimping tool T. As described above, deformation of the portion 1208′ of the outer side surface 1208 of the substantially tube-shaped body 1202 of the core member 1200 results in a portion D₁₂₁₂′ of the passage diameter D₁₂₁₂ extending along the intermediate portion L₁₂₀₀′ of the length L₁₂₀₀ of the substantially tube-shaped body 1202 also being deformed, whereby the portion 1210′ of the inner surface 1210 of the substantially tube-shaped body 1202 of the core member 1200 is joined to the portion 1022′ of the outer surface 1022 of the body 1016 of the cannula 1012.

In some configurations, the crimping tool T may punch, crimp, swage or materially deform, for example, a portion of the outer surface 1208 of the substantially tube-shaped body 1202 of the core member 1200. Accordingly, in such implementations, the portion 1208′ of the outer surface 1208 of the substantially tube-shaped body 1202 may define crimping pockets formed by the material deformation of the outer surface 1208 of the substantially tube-shaped body 1202. The material deformation of the outer surface 1208 of the portion of the substantially tube-shaped body 1202 distinguishes a “deformed” core member 1200 that is mechanically connected to the cannula 1012 from a virgin or “non-deformed” core member 1200 (see, e.g., FIGS. 52C-52D) having an outer surface 1208 of the substantially tube-shaped body 1202 that does not define crimping pockets 1208′. Furthermore, after non-removably-securing the core portion 1200 to the cannula 1012 as described above, the core portion 1200 is not permitted to axially shift relative the cannula such that the dimension defined by the predetermined hub-core distance D₁₀₁₄₋₁₂₀₀ extending between the distal end surface 1038 of the hub 1014 and the proximal end surface 1204 of the core member 1200 is fixed.

With reference to FIGS. 52F-52H, after the core member 1200 has been joined to the cannula 1012 to define the second sub-assembly that defines the predetermined hub-core distance D₁₀₁₄₋₁₂₀₀ extending between the distal end surface 1038 of the hub 1014 and the proximal end surface 1204 of the core member 1200, the cannula carrier 1100 is then joined to the core member 1200 to further define the third sub-assembly, which may be alternatively referred to as the hypodermic interface assembly 1000. Thereafter, as seen at FIG. 56A, the hypodermic interface assembly 1000 may be joined to an injection gun I. As will be described in the following disclosure at FIGS. 54A-54C and 56C-56G, the second portion 1000b of the hypodermic interface assembly 1000 is configured to controllably and predictably separate from the first portion 1000a of the hypodermic interface assembly 1000 as a result of the hypodermic interface assembly 1000 including the predetermined hub-core distance D₁₀₁₄₋₁₂₀₀ extending between the distal end surface 1038 of the hub 1014 and the proximal end surface 1204 of the core member 1200.

As seen at FIG. 52F, the cannula carrier 1100 may be arranged opposite the distal end 1016_{D} of the tube-shaped body 1016 of the cannula 1012 such that the central axis A₁₁₀₀-A₁₁₀₀ of the cannula carrier 1110 is axially aligned with the central axis A₁₀₁₂-A₁₀₁₂ of the cannula 1012. The central axes A₁₀₁₂-A₁₀₁₂ and A₁₁₀₀-A₁₁₀₀ of each of the cannula 1012 and the cannula carrier 1100 correspond to the central axis A₁₀₀₀-A₁₀₀₀ of the hypodermic interface assembly 1000.

The outer surface 1022 of the tube-shaped body 1016 of the cannula 1012 that defines the outer diameter of the cannula 1012 is less than each of the first passage diameter D₁₁₁₆ₐ, the second passage diameter D_{1116b}, and the third passage diameter D_{1116c} extending, respectively, through the first passage portion 1116a, the second passage portion 1116b, and the intermediate passage portion 1116c of the axial passage 1116 extending through the cannula carrier 1100. Accordingly, as the cannula carrier 1100 is subsequently axially advanced in a direction toward the distal end surface 1038 of the hub 1014, the distal end 1016_{D} of the tube-shaped body 1016 of the cannula 1012 is permitted to pass through the axial passage 1116 of the cannula carrier 1100 such that, as seen at FIG. 52G, the distal end 1016_{D} of the tube-shaped body 1016 of the cannula 1012 may be subsequently arranged beyond the distal end surface 1110 of the cannula carrier 1100.

Once the distal end 1016_{D} of the tube-shaped body 1016 of the cannula 1012 is arranged beyond the distal end surface 1110 of the cannula carrier 1100, the cannula carrier 1100 is then further axially advanced in a direction toward the distal end surface 1038 of the hub 1014 such that the length L₁₂₀₀ of the substantially tube-shaped body 1202 of the core member 1200 may be subsequently arranged within the axial passage 1116 extending through the cannula carrier 1100 as seen at FIG. 52G. As explained above at FIG. 53, the second passage diameter D_{1116b} defined by the second passage portion 1116b of the axial passage 1116 extending through the cannula carrier 1100 is approximately equal to but slightly greater than the outer diameter D₁₂₀₈ of the defined by the outer surface 1208 of the substantially tube-shaped body 1202 of the core member 1200. Accordingly, with reference to FIGS. 52G-52H, as the cannula carrier 1100 is further axially advanced in a direction toward the distal end surface 1038 of the hub 1014, the distal portion L_{1200b} of the length L₁₂₀₀ of the substantially tube-shaped body 1202 of the core member 1200 is arranged outside of the axial passage 1116 extending through the body 1106 of the head portion 1102 of the cannula carrier 1100 and beyond the distal end surface 1110 of the body 1106 of the head portion 1102 of the cannula carrier 1100.

Although the distal portion L_{1200b} of the length L₁₂₀₀ of the substantially tube-shaped body 1202 of the core member 1200 is arranged beyond the distal end surface 1110 of the cannula carrier 1100, the proximal portion L₁₂₀₀ₐ of the length L₁₂₀₀ of the substantially tube-shaped body 1202 of the core member 1200 is contained within the axial passage 1116 extending through the cannula carrier 1100 and does not extend beyond the distal end surface 1110 of the cannula carrier 1100. As explained above at FIG. 53, the first passage diameter D₁₁₁₆ₐ defined by the first passage portion 1116a of the axial passage 1116 extending through the cannula carrier 1100 is approximately equal to but slightly less than the substantially constant diameter D₁₂₂₀ₐ of the first outer side surface portion 1220a of the outer side surface 1220 of the cannula-carrier-engaging portion 1218 of the substantially tube-shaped body 1202 of the core member 1200. Accordingly, as seen at FIGS. 52H and 53, the first outer side surface portion 1220a of the outer side surface 1220 of the cannula-carrier-engaging portion 1218 comes into contact with and engages one, one or more, or all of: the first inner surface portion 1112a of the inner surface 1112 of the body 1106 of the head portion 1102 of the cannula carrier 1100; the second inner surface portion 1112b of the inner surface 1112 of the body 1106 of the head portion 1102 of the cannula carrier 1100; and the intermediate inner surface portion 1112c of the inner surface 1112 of the body 1106 of the head portion 1102 of the cannula carrier 1100. As a result, the cannula-carrier-engaging portion 1218 of the core member 1200 becomes friction-fit coupled to one, one or more, or all of: the first inner surface portion 1112a; the second inner surface portion 1112b; and the intermediate inner surface portion 1112c of the inner surface 1112 of the body 1106 of the head portion 1102 of the cannula carrier 1100, thereby “plugging” or “fluidly sealing” the distal opening 1120 of the cannula carrier 1100.

With reference to FIG. 52G, prior to the first outer side surface portion 1220a of the outer side surface 1220 of the cannula-carrier-engaging portion 1218 of the core member 1200 coming into contact with and engaging the first inner surface portion 1112a of the inner surface 1112 of the body 1106 of the head portion 1102 of the cannula carrier 1100, each leg portion 1104a, 1104b, 1104c, 1104d of the plurality of leg portions 1104 slides and/or flexes over the first outer side surface portion 1220a of the outer side surface 1220 of the cannula-carrier-engaging portion 1218 of the core member 1200. Once the first outer side surface portion 1220a of the outer side surface 1220 of the cannula-carrier-engaging portion 1218 of the core member 1200 is arranged beyond the proximal end surface 1108 of the body 1106 of the head portion 1102 of the cannula carrier 1100 such that the first outer side surface portion 1220a of the outer side surface 1220 of the cannula-carrier-engaging portion 1218 is arranged within the first passage portion 1116a of the axial passage 1116 extending through the cannula carrier 1100, the first outer side surface portion 1220a of the outer side surface 1220 of the cannula-carrier-engaging portion 1218 may then begin to frictionally-engage one, one or more, or all of: the first inner surface portion 1112a; the second inner surface portion 1112b; and the intermediate inner surface portion 1112c of the inner surface 1112 of the body 1106 of the head portion 1102 of the cannula carrier 1100.

With reference to FIG. 53, because the substantially constant diameter D₁₂₂₀ₐ of the first outer side surface portion 1220a of the outer side surface 1220 of the cannula-carrier-engaging portion 1218 of the substantially tube-shaped body 1202 of the core member 1200 is greater than the first passage diameter D₁₁₁₆ₐ defined by the first passage portion 1116a of the axial passage 1116 extending through the cannula carrier 1100, one or both of the first outer side surface portion 1220a and the second side surface portion 1220b of the outer side surface 1220 of the cannula-carrier-engaging portion 1218 may, for example, plastically deform a portion of one, one or more, or all of: the first inner surface portion 1112a; the second inner surface portion 1112b; and the intermediate inner surface portion 1112c of the inner surface 1112 of the body 1106 of the head portion 1102 of the cannula carrier 1100, thereby creating a radially-projecting annular recess 1112′ in one, one or more, or all of: the first inner surface portion 1112a; the second inner surface portion 1112b; and the intermediate inner surface portion 1112c of the inner surface 1112 of the body 1106 of the head portion 1102 of the cannula carrier 1100 that is configured to receive the cannula-carrier-engaging portion 1218 of the core member 1200 in a friction-fit configuration. Alternatively, in some configurations, the radially-projecting annular recess 1112′ may be pre-formed in one, one or more, or all of: the first inner surface portion 1112a; the second inner surface portion 1112b; and the intermediate inner surface portion 1112c of the inner surface 1112 of the body 1106 of the head portion 1102 of the cannula carrier 1100 such that the cannula-carrier-engaging portion 1218 of the core member 1200 may be snap-fit-received within the radially-projecting annular recess 1112′. Irrespective of the friction-fit or snap-fit joining of the cannula-carrier-engaging portion 1218 of the core member 1200 with the radially-projecting annular recess 1112′, once the cannula-carrier-engaging portion 1218 of the core member 1200 is joined to the radially-projecting annular recess 1112′ of the cannula carrier 1100, the third sub-assembly, which may be alternatively referred to as the hypodermic interface assembly 1000, may then be said to be formed.

Furthermore, as seen at FIG. 52H, once the cannula-carrier-engaging portion 1218 of the core member 1200 is joined to the radially-projecting annular recess 1112′ of the cannula carrier 1100, each proximal end surface 1122 of each leg portion 1104a, 1104b, 1104c, 1104d of the plurality of leg portions 1104 of the cannula carrier 1100 may be configured to matingly-couple with or be matingly-disposed-adjacent the outer shoulder surface portion 1064 defined by the outer surface 1040 of the substantially tube-shaped body 1034 of the hub 1014. Accordingly, although the each proximal end surface 1122 of each leg portion 1104a, 1104b, 1104c, 1104d of the plurality of leg portions 1104 may be configured to matingly-couple with or be matingly-disposed-adjacent the outer shoulder surface portion 1064 defined by the outer surface 1040 of the substantially tube-shaped body 1034 of the hub 1014, each proximal end surface 1122 of each leg portion 1104a, 1104b, 1104c, 1104d of the plurality of leg portions 1104 may not be mechanically interlocked with the outer shoulder surface portion 1064 defined by the outer surface 1040 of the substantially tube-shaped body 1034 of the hub 1014. Accordingly, in some configurations, the outer shoulder surface portion 1064 is shaped to prevent further axial movement of the cannula carrier 1100 in a proximal direction relative the core member 1200 as the cannula carrier 1100 is axially sleeved over the core member 1200 as described above at FIGS. 52F-52H).

Referring to FIG. 54A, with the cannula carrier 1100 non-removably-joined to the core member 1200 as described above at FIGS. 52F-52H and 53, the hypodermic interface assembly 1000 is said to be fully assembled. As seen at FIG. 54A, a first portion L₁₀₁₂ₐ (see also, e.g., FIG. 52H) of a length of the cannula 1012 is arranged within and contained by passages 1044, 1212, and 1116 of, respectively, the hub 1014, the core member 1200, and the cannula carrier 1100. More specifically, the first portion L₁₀₁₂ₐ (see also, e.g., FIG. 52H) of the length of the cannula 1012 extends between the proximal end surface 1018 at the proximal end 1016_{P} of the tube-shaped body 1016 of the cannula 1012 (that may be arranged within the second passage portion 1044b of the passage 1044 of the hub 1014) and the distal end surface 1206 of the core member 1200.

With continued reference to FIG. 54A, after fully assembling the hypodermic interface assembly 1000, the cannula 1012 is also defined by another (i.e., a second) length portion that distally extends beyond the distal end surface 1206 of the core member 1200. More specifically, a second portion L_{1012b} (see also FIG. 43) of the length of the cannula 1012 extends between the distal end surface 1206 of the core member 1200 and the sharp piercing tip 1032 at the distal end 1016_{D} of the tube-shaped body 1016 of the cannula 1012. Furthermore, unlike the first portion L₁₀₁₂ₐ of the length of the cannula 1012, the second portion L_{1012b} of the length of the cannula 1012 is not arranged within and is not contained by the passages 1044, 1212, and 1116 of, respectively, the hub 1014, the core member 1200, and the cannula carrier 1100.

As seen at FIG. 54A, once the hypodermic interface assembly 1000 is assembled as described above at FIGS. 52H and 53, the hypodermic interface assembly 1000 may be subsequently attached to injection gun I (see also, e.g., FIG. 56A) in an “at-rest orientation”. At FIG. 54B, the hypodermic interface assembly 1000 is shown at a “biased orientation” (after, as seen at FIG. 56D, the cannula 1012 is subjected to the one or more radial forces X_{R} relative to the central axis A₁₀₀₀-A₁₀₀₀ extending through the hypodermic interface assembly 1000). Thereafter, as seen at FIG. 54C, the hypodermic interface assembly 1000 is shown arranged in a “separated orientation” defined by the first portion 1000a of the hypodermic interface assembly 1000 that is configured to remain attached to an injection gun I and the second portion 1000b of the hypodermic interface assembly 1000 that separates from the injection gun I and is configured to be removed from an impaled orientation within the flesh of the animalia S (as seen at FIGS. 56E-56G).

With reference to FIG. 54C, the cannula 1012 may be further defined by a proximal segment 1012_{P}, an intermediate segment 1012_{I}, and a distal segment 1012_{D}. The proximal segment 1012, of the cannula 1012 and the intermediate segment 1012_{I} of the cannula 1012 extend along and define the first portion L₁₀₁₂ₐ of the length of the cannula 1012 whereas the distal segment 1012_{D} pf the cannula 1012 extends along and defines the second portion L_{1012b} of the length of the cannula 1012. When the cannula 1012 is “broken” as a result of the hypodermic interface assembly 1000 being arranged in the “separated orientation” as seen at FIG. 54A, the proximal segment 1012_{P} of the cannula 1012 may generally define a broken distal end 1012_{P-D} that is separated from a broken proximal end 1012_{I-P} defined by the intermediate segment 1012_{I} of the cannula 1012. In some configurations, the broken distal end 1012_{P-D} of the proximal segment 1012_{P} of the cannula 1012 may be: aligned with the distal end surface 1038 of the hub 1014; arranged distally of the distal end surface 1038 of the hub 1014; or arranged proximally of the distal end surface 1038 of the hub 1014. Preferably, the broken distal end 1012_{P-D} is located in the intermediate segment 10121 distal to the distal end surface 1038 of the hub 1014 and proximal to the proximal end surface 1204 of the core member 1200.

Furthermore, as seen at FIG. 54C, the proximal segment 1012_{P} of the cannula 1012 is a component of the first portion 1000a of the hypodermic interface assembly 1000 that is configured to remain attached to an injection gun I whereas the intermediate segment 1012_{I} of the cannula 1012 and the distal segment 1012_{D} of the cannula 1012 define components of the second portion 1000b of the hypodermic interface assembly 1000 that is configured to separate from the injection gun I. Furthermore, as will be described in the following disclosure, the distal segment 1012_{D} pf the cannula 1012, which also defines the second portion L_{1012b} of the length of the cannula 1012 is configured to be impaled within the flesh of the animalia S after the second portion 1000b of the hypodermic interface assembly 1000 separates from the injection gun I.

As shown at FIG. 54C, for example, the location where the cannula 1012 is broken (i.e., the location along the length 1012ₐ of cannula 1012 where the broken distal end 1012_{P-D} is separated from the broken proximal end 1012_{I-P}) is predictably-controlled as a result of the predetermined hub-core distance D₁₀₁₄₋₁₂₀₀ (see, e.g., FIG. 54A) extending between the distal end surface 1038 of the hub 1014 and the proximal end surface 1204 of the core member 1200. The predetermined hub-core distance D₁₀₁₄₋₁₂₀₀ extending between the distal end surface 1038 of the hub 1014 and the proximal end surface 1204 of the core member 1200 arises from a predetermined placement of the core member 1200 relative the cannula 1012 as seen at FIG. 52D (and then subsequent crimping of the outer surface 1208 of the substantially tube-shaped body 1202 of the core member 1200 as seen at FIGS. 52D-52E).

Referring now to FIGS. 55 and 56A-56G, a methodology for utilizing the hypodermic interface assemblies 1000 is shown. As described above, the design of the hypodermic interface assembly 1000 promotes controlled and predicable separation (see, e.g., FIG. 54A-54C) of a second portion of the cannula 1012 (that is defined by the intermediate segment 1012_{I} of the cannula 1012 and the distal segment 1012_{D} of the cannula 1012 as seen at, e.g., FIG. 54C), the core member 1200, and the cannula carrier 1100 (that collectively define the second portion 1000b of the hypodermic interface assembly 1000) relative to a first portion of the cannula 1012 (that is defined by the proximal segment 1012_{P} of the cannula 1012 as seen at, e.g., FIG. 54C) and the hub 1014 (that collectively define the first portion 1000a of the hypodermic interface assembly 1000).

In some instances, predictable and controlled separation of the second portion 1000b of the hypodermic interface assembly 1000 from the first portion 1000a of the hypodermic interface assembly 1000 may occur after the cannula 1012 pierces the subject S (see, e.g., FIGS. 56A-56B). The subject S may be, for example, animalia, such as a human or non-human (i.e., an animal such as, for example, pig or swine). In other examples, the subject S may be an inanimate object. The predicable and controlled separation of the second portion 1000b of the hypodermic interface assembly 1000 from the first portion 1000a of the hypodermic interface assembly 1000 mitigates separation of the proximal segment 1012_{P} cannula 1012 from the entirety of the hub 1014, which may otherwise undesirably result in the entirety of the cannula 1012 being broken-off and subsequently lost (or make difficult easily locating the broken-off cannula 1012) within the flesh of the animalia.

Referring to FIG. 55, the hypodermic interface assembly 1000 is shown connected to an injecting device I, such as, for example, an injection gun. The hypodermic interface assembly 1000 may be connected to a barrel portion I_{B} of the injection gun I by arranging, for example, the first radially-outward projection or ear 1056 and the second radially-outward projection or ear 1058 extending from the of the barrel-engaging portion 1050 that extends from the outer surface 1040 of the substantially tube-shaped body 1034 of the hub 1014 in corresponding recesses (not shown) formed by the barrel portion I_{B} of the injection gun I and then, for example, quarter-turn locking the hypodermic interface assembly 1000 for removably-securing the first radially-outward projection or ear 1056 and the second radially-outward projection or ear 1058 extending from the of the barrel-engaging portion 1050 to the barrel portion I_{B} of the injection gun I.

The injection gun I may include a fluid container C that contains a fluid F (see also, e.g., FIGS. 56C-56D). The fluid F may be metered from: (1) the container C; (2) through the injection gun I; (3) into the hypodermic interface assembly 1000; and (4) out of the hypodermic interface assembly 1000 and into the flesh of the subject S. The injection gun I may be actuated when a user U presses, for example, an actuator I_{A} such as, for example, a trigger in order to cause movement of the fluid F as described above. The injection gun I may be powered in any desirable manner such as, for example: battery powered; air powered; manually powered; or a combination thereof.

Referring to FIG. 56A, the user may grasp the injection gun I and position the sharp piercing tip 1032 formed by the distal end surface 1020 of the tube-shaped body 1016 of the cannula 1012 near the outer surface S_{S} of the subject S, which may define the skin or hide of the subject S. Referring to FIGS. 56A-56B, the user U may impart an axial force according to the direction of the arrow X_{A} to the injection gun I along the central axis A₁₀₀₀-A₁₀₀₀ extending through the hypodermic interface assembly 1000 such that the sharp piercing tip 1032 formed by the distal end surface 1020 of the tube-shaped body 1016 of the cannula 1012 axially pierces the outer surface S_{S} of the subject S.

As described above, the distal end surface 1206 of the substantially tube-shaped body 1202 of the core member 1200 (which is a distal-most portion of the substantially tube-shaped body 1202 of the core member 1200) is arranged distally beyond the distal end surface 1110 of the cannula carrier 1100. Accordingly, as seen at FIGS. 56B-56F, when the hypodermic interface assembly 1000 is interfaced with the outer surface S_{S} of the subject S, the distal end surface 1206 of the substantially tube-shaped body 1202 of the core member 1200 may be disposed adjacent the outer surface S_{S} of the subject S whereas the distal end surface 1110 of the cannula carrier 1100 does not engage the outer surface S_{S} of the subject S, e.g., the distal end surface 1110 of the cannula carrier 1100 is spaced away from the outer surface S_{S} of the subject S at a distance according to the distal portion L_{1200b} of the length L₁₂₀₀ of the substantially tube-shaped body 1202 of the core member 1200.

Referring to FIG. 56C, after the outer surface S_{S} of the subject S has been axially pierced by the cannula 1012, the user U may optionally actuate the actuator I_{A} in order to cause movement of the fluid F from: (1) the container C; (2) through the injection gun I; (3) into the hypodermic interface assembly 1000; and (4) out of the hypodermic interface assembly 1000 and into the flesh of the subject S. In an example, the fluid F may firstly enter the hypodermic interface assembly 1000 from the injection gun I at the passage 1044 formed by the substantially tube-shaped body 1034 of the hub 1014 by way of the proximal opening 1046 formed by the proximal end surface 1036 of the substantially tube-shaped body 1034 of the hub 1014. Then, the fluid F may secondly enter the passage 1026 extending through the tube-shaped body 1016 of the cannula 1012 by way of the proximal opening 1028 formed by the proximal end surface 1018 of the body 1016 of the cannula 1012. Then, thirdly, the fluid F may exit the passage 1026 extending through the tube-shaped body 1016 of the cannula 1012 by way of the distal opening 1030 formed by the distal end surface 1020 of the body 1016 of the cannula 1012.

The fluid F may be any desirable composition that is intended to be delivered to the animalia S. In some instances, the fluid F may be a medicament, a pharmaceutical, a vaccine, an anesthetic, or the like. Accordingly, the fluid F may not include any type of fluid that is not intended to be injected into animalia S. Although the hypodermic interface assembly 1000 also may be utilized for injecting fluid F into animalia S, the hypodermic interface assembly 1000 may be utilized for removing fluid F (e.g., blood) from animalia S. Therefore, it will be appreciated that the hypodermic interface assembly 1000 may deliver or receive fluid F.

Referring to FIGS. 54C and 56D, after the outer surface S_{S} of the subject S has been axially pierced by the cannula 1012, the subject S may experience discomfort, e.g., as a result of pain arising from the outer surface S_{S} being pierced by the sharp piercing tip 1032 formed by the distal end surface 1020 of the tube-shaped body 1016 of the cannula 1012. Accordingly, if the user U is sufficiently grasping the injection gun I, any movement of the subject S may result in the cannula 1012 being subjected to one or more radial forces X_{R} relative the central axis A₁₀₀₀-A₁₀₀₀ extending through the hypodermic interface assembly 1000 that may cause the cannula 1012 to bend, warp, or crease, such that the central axis A₁₀₁₂-A₁₀₁₂ extending through the axial center of the tube-shaped body 1016 of a portion of the cannula 1012 is not coincident with the central axis A₁₀₀₀-A₁₀₀₀ extending through the hypodermic interface assembly 1000 that may be coaxially aligned with the other components of the hypodermic interface assembly 1000 such as, for example, the hub 1014, the cannula carrier 1100, and the core member 1200.

If one or more forces X_{R} (that are normal to the central axis A₁₀₀₀-A₁₀₀₀ of the hypodermic interface assembly 1000) are applied to the distal portion of the cannula 1012, then, the cannula 1012 may flex. As the one or more forces X_{R} increases, at a certain point, the material from which the cannula 1012 is made (e.g., metal) begins to yield, the cannula 1012 flexing and transitioning to a non-axial bent or warped shape, i.e., the bent or warped cannula 1012 will not return back to its original un-flexed, un-bent, and un-warped state (unless further forces are applied to bend the material back to its original state). When a cannula 1012 will not return to its original un-flexed, un-bent, and un-warped state, it is in a condition termed “plastic deformation”. If the one or more forces X_{R} are insufficient to cause plastic deformation of the cannula 1012, when the one or more forces X_{R} are released the cannula 1012 returns to its original (un-flexed) shape.

A most probable area of maximum plastic deformation (from stresses caused by one or more forces X_{R}) has a center that is located distal to the distal end surface 1038 of the hub 1014, approximately the same distance as the outside radius of the cannula 1012 (see, e.g., r_{can} at FIG. 54A; i.e., one-half of outer diameter D₁₀₁₂ of the cannula 1012), and about the thickness T₁₀₁₂ of the body 1016 of the cannula 1012 into the cannula. The highest probability area for maximum plastic deformation (caused by one or more forces X_{R}) is approximately the cannula radius r_{can} in length and approximately twice the thickness T₁₀₁₂ extending between the outer surface 1022 of the body 1016 of the cannula 1012 and the inner surface 1024 of the body 1016 of the cannula 1012.

After plastic deformation has occurred, the cannula 1012 is weakened at the point of bending/warping because the atomic lattice structure of the material (e.g., metal) of the cannula 1012 has been disturbed. At around 45° (+/−15%) of bending relative to the central axis A₁₀₀₀-A₁₀₀₀ of the hypodermic interface assembly 1000, the atomic lattice becomes so distorted that the plastic deformation may lead to the formation of a crease in the cannula 1012. A crease represents a highly weakened area of the cannula 1012.

Because both of the hub 1014 and the core member 1200 of the hypodermic interface assembly 1000 may be formed from a non-flexible or substantially rigid material (e.g., metal), any stresses imparted to the cannula 1012 arising from the one or more radial forces X_{R} may be transmitted from a distal portion of the non-flexible or substantially rigid materials defining the second portion 1000b of the hypodermic interface assembly 1000 (which may be defined by, for example, the distal end surface 1206 of the distal-most portion of the substantially tube-shaped body 1202 of the core member 1200 that is disposed adjacent the outer surface S_{S} of the subject S) toward the most probable area of maximum plastic deformation. That is, the stresses from the one or more radial forces X_{R} may be transmitted to a predetermined proximal region of the cannula 1012 (which may be defined by, for example, the broken proximal end 1012_{I-P} of the intermediate segment 1012_{I} of the cannula 1012 and the broken distal end 1012_{P-D} of the proximal segment 1012_{P} of the cannula 1012 as seen at, e.g., FIG. 54C) that is arranged within or near a region bound by the predetermined hub-core distance D₁₀₁₄₋₁₂₀₀ extending between the distal end surface 1038 of the hub 1014 and the proximal end surface 1204 of the core member 1200.

Because the stresses from the one or more radial forces X_{R} are transmitted to, substantially to, about, along, or on the proximal region of the cannula 1012 that is bound by the predetermined hub-core distance D₁₀₁₄₋₁₂₀₀ (see, e.g., FIG. 54A), the cannula 1012 bends, warps, and/or creases at, substantially at, about, along, or on the region bound by the predetermined hub-core distance D₁₀₁₄₋₁₂₀₀, and the cannula 1012 then may break (see, e.g., FIG. 54B) in the proximal region of the cannula 1012 that is bound by the predetermined hub-core distance D₁₀₁₄₋₁₂₀₀, and the cannula carrier 1100 is separated (see, e.g., FIG. 54C) from the hub 1014.

As seen at FIG. 56D, as a result of stresses transmitted from distal portion of the non-flexible or substantially rigid materials defining the second portion 1000b of the hypodermic interface assembly 1000 toward the proximal region of the cannula 1012 that is arranged within or near the predetermined hub-core distance D₁₀₁₄₋₁₂₀₀ extending between the distal end surface 1038 of the hub 1014 and the proximal end surface 1204 of the core member 1200, the cannula 1012 may be permitted to also bend or deviate away from the central axis A₁₀₀₀-A₁₀₀₀ extending through the hypodermic interface assembly 1000 (see, e.g., the axes A₁₀₁₂-A₁₀₁₂, A₁₁₀₀-A₁₁₀₀, A₁₂₀₀-A₁₂₀₀ of the cannula 1012, the cannula carrier 1100, and the core member 1200). Accordingly, the axes A₁₀₁₂-A₁₀₁₂, A₁₀₀₀-A₁₀₀₀, A₁₂₀₀-A₁₂₀₀ of the cannula 1012, the cannula carrier 1100, and the core member 1200 generally deviate away from the axis A₁₀₁₄-A₁₀₁₄ of the hub 1014, which may remain coincident with the central axis A₁₀₀₀-A₁₀₀₀ extending through the hypodermic interface assembly 1000.

Referring to FIGS. 54C and 56E, the stresses transmitted from the distal portion of the non-flexible or substantially rigid materials defining the second portion 1000b of the hypodermic interface assembly 1000 toward the proximal region of the cannula 1012 that is arranged within or near the predetermined hub-core distance D₁₀₁₄₋₁₂₀₀ extending between the distal end surface 1038 of the hub 1014 and the proximal end surface 1204 of the core member 1200 may continue to bend the cannula 1012 relative the hub 1014 until the second portion of the cannula 1012 (that is defined by the intermediate segment 1012_{I} of the cannula 1012 and the distal segment 1012D of the cannula 1012 as seen at, e.g., FIG. 54C) that is connected to the core member 1200 and the cannula carrier 1100 controllably separates from the hub 1014. For example, the second portion of the cannula 1012 is disconnected from the first portion of the cannula 1012 that is defined by the proximal segment 1012_{P} of the cannula 1012 (as well as disconnecting or separating each leg portion 1104a, 1104b, 1104c, 1104d of the cannula carrier 1100 from the outer shoulder surface portion 1064 defined by the outer surface 1040 of the substantially tube-shaped body 1034 of the hub 1014). As a result, the second portion 1000b of the hypodermic interface assembly 1000 including the cannula carrier 1100, the core member 1200, and the second portion of the cannula 1012 predictably and controllably separates from the first portion 1000a of the hypodermic interface assembly 1000 defined by the first portion of the cannula 1012 and the hub 1014 (at, or substantially at, about, along, or a proximal region of the cannula 1012 arranged within or near the predetermined hub-core distance D₁₀₁₄₋₁₂₀₀ extending between the distal end surface 1038 of the hub 1014 and the proximal end surface 1204 of the core member 1200).

When the one or more forces X_{R} are applied to the cannula 1012, an initial concentration of the one or more forces X_{R} resides in the most probable area of maximum plastic deformation as described above (i.e., relatively near to the distal end surface 1038 of the hub 1014). That is, the stresses from the one or more radial forces X_{R} may be transmitted to the most probable area of maximum plastic deformation for plastic deformation of the cannula 1012. As the one or more forces X_{R} applied to cannula 1012 increase (and as bending proceeds), plastic deformation continues and eventually may lead to the formation of a crease in the most probable area of maximum plastic deformation.

However, if during bending or plastic deformation of the cannula 1012 the proximal end surface 1204 of the core member 1200 comes into contact with the distal end surface 1038 of the hub 1014 (e.g., the proximal end surface 1204 of the core member 1200 comes into contact with the corner of the hub 1014, that is, where the distal end surface 1038 of the hub 1014 meets the outer surface 1040 defining the second portion 1034b of the substantially tube-shaped body 1034), it is possible that continued bending or plastic deformation in the most probable area of maximum plastic deformation is prevented because this contact between the hub 1014 and the core member 1200 may effectively brace the most probable area of maximum plastic deformation. If such bracing occurs, is some implementations, the stresses from the one or more radial forces X_{R} may be transmitted from the most probable area of maximum plastic deformation to the distal side of the core member 1200 (i.e., the one or more radial forces X_{R} may be transmitted to a portion of the cannula 1012 that is distal to the distal end surface 1206 of the core member 1200). Further bending or plastic deformation of the cannula 1012 after such bracing may result in breaking of the cannula 1012 and separation of the cannula 1012 both from the core member 1200 and from the hub 1014. These predicted results are consistent with failures on the distal side of the core member 1200 after contact between the hub 1014 and the core member 1200 occurs. As discussed herein, such breakage of the cannula 1012 on the distal side of the core member 1200 during an injection is undesirable because the cannula 1012 sticking out of an animal is difficult to see, difficult to retrieve, and further may be able to migrate inside the body of the animalia.

As such, it is desirable to arrange the components of the hypodermic interface assembly 1000 such that the second portion 1000b of the hypodermic interface assembly 1000 is predictably and controllably separated from the first portion 1000a of the hypodermic interface assembly 1000. For example, the predetermined hub-core distance D₁₀₁₄₋₁₂₀₀ as seen at FIG. 54A is a minimum distance needed so that bracing does not occur. In particular, the predetermined hub-core distance D₁₀₁₄₋₁₂₀₀ should be large enough so that bracing does not begin between the hub 1014 and the core member 1200 during deformation of the cannula 1012. In one embodiment, when both the distal end surface 1038 of the hub 1014 and the proximal end surface 1204 of the core member 1200 are flat and are normal to the central axis A₁₀₀₀-A₁₀₀₀, plus the outside radius of the distal end surface 1038 of the hub rho (i.e., one-half of the outer diameter of the distal end surface 1038 of the hub 1014) and outside radius of the proximal end surface 1204 of the core member r_{core member} (i.e., one-half of the outer diameter of the proximal end surface 1204 of the core member 1200) are similar (+/−33%), then the predetermined hub-core distance D₁₀₁₄₋₁₂₀₀ would be approximately equal to the outside radius of the hub rho plus the outside radius of the cannula rₒₐₙ. In another embodiment, when one or both the distal end surface 1038 of the hub 1014 and the proximal end surface 1204 of the core member 1200 are not flat, then predetermined hub-core distance D₁₀₁₄₋₁₂₀₀ may be the average distance between distal end surface 1038 of the hub 1014 and proximal end surface 1204 of the core member 1200. In one embodiment, the minimum predetermined hub-core distance D₁₀₁₄₋₁₂₀₀ is a function of the gauge of the cannula (e.g., 20, 18, 16, or 14 gauge), the outside radius of the distal end surface 1038 of the hub rho, and outside radius of the proximal end surface 1204 of the core member r_{core member}.

In some implementations: for a 20 gauge cannula, the outside radius of the proximal end surface 1204 of the core member r_{core member} may be about 2.00 mmm, the outside radius of the distal end surface 1038 of the hub r_{hub}, may be about 1.77 mm, and the predetermined hub-core distance D₁₀₁₄₋₁₂₀₀ may be about 1.50 mm; for a 18 gauge cannula, the outside radius of the proximal end surface 1204 of the core member r_{core member} may be about 2.11 mmm, the outside radius of the distal end surface 1038 of the hub r_{hub}, may be about 1.91 mm, and the predetermined hub-core distance D₁₀₁₄₋₁₂₀₀ may be about 1.75 mm; for a 16 gauge cannula, the outside radius of the proximal end surface 1204 of the core member r_{core member} may be about 2.16 mmm, the outside radius of the distal end surface 1038 of the hub r_{hub}, may be about 1.95 mm, and the predetermined hub-core distance D₁₀₁₄₋₁₂₀₀ may be about 1.84 mm; and for a 14 gauge cannula, the outside radius of the proximal end surface 1204 of the core member r_{core member} may be about 2.20 mmm, the outside radius of the distal end surface 1038 of the hub rho, may be about 1.98 mm, and the predetermined hub-core distance D₁₀₁₄₋₁₂₀₀ may be about 1.91 mm. Further, in some implementations, for cannulas ranging from 20 gauge to 14 gauge, the predetermined hub-core distance D₁₀₁₄₋₁₂₀₀ may range from, for example: about 1.50 mm to about 4.00 mm; about 1.55 mm to about 3.00 mm; about 1.60 mm to about 2.50 mm; or about 1.60 mm to about 2.00 mm In other preferred implementations, the predetermined distance D₁₀₁₄₋₁₂₀₀ may be equal to or greater than about 2.00 mm.

After the controlled separation of the second portion 1000b of the hypodermic interface assembly 1000 from the first portion 1000a of the hypodermic interface assembly 1000, one or more of leg portion 1104a, 1104b, 1104c, 1104d of the cannula carrier 1100 may deform, expand, or splay outwardly, which may increase visibility to a user to assist in locating where the cannula 1012 is impaled within the flesh of the animalia S.

As seen at FIG. 56E, because the cannula carrier 1100 is non-separably joined to the second portion of the cannula 1012 by way of the core member 1200, the user U, may easily identify a location of the animalia S where the distal segment 1012_{D} of the cannula 1012 is impaled within the flesh of the animalia S. Furthermore, the distal end surface 1206 of the substantially tube-shaped body 1202 of the core member 1200 remains disposed adjacent the outer surface S_{S} of the subject S while the distal end surface 1110 of the cannula carrier 1100 still does not engage and is spaced away the outer surface S_{S} of the subject S at a distance according to the distal portion L_{1200b} of the length L₁₂₀₀ of the substantially tube-shaped body 1202 of the core member 1200. As a result, in addition to the cannula carrier 1100, the core member 1200 is also visible to the user U. Therefore, the location of the animalia S where the distal segment 1012_{D} of the cannula 1012 is impaled within the flesh of the animalia S is easily identifiable as a result of, for example: (1) the distal end surface 1206 of the substantially tube-shaped body 1202 of the core member 1200 directly resting upon the skin S_{S} or hide of the animalia S; and (2) the cannula carrier 1100 of the second portion 1000b of the hypodermic interface assembly 1000 being connected to the core member 1200 and arranged at a distance slightly away from the skin S_{S} or hide of the animalia S (while the distal segment 1012D of the cannula 1012 is not visible to the user U since the distal segment 1012_{D} of the cannula 1012 is contained within and obscured by the flesh of the animalia S).

Thereafter, as seen at FIG. 56F, the user U may pinch or grasp the second portion 1000b of the hypodermic interface assembly 1000 and apply a pulling force to the second portion 1000b of the hypodermic interface assembly 1000 (that also includes the impaled distal segment 1012_{D} of the cannula 1012). As seen at FIG. 56G, as a result of the pulling force to the second portion 1000b of the hypodermic interface assembly 1000 by the user U, the distal segment 1012_{D} of the cannula 1012 is removed from the flesh of the animalia S such that the distal segment 1012_{D} of the cannula 1012 is not lost or would therefore otherwise undesirably remain within the flesh of the animalia S.

A number of implementations have been described. Nevertheless, it will be understood that various modifications may be made without departing from the spirit and scope of the disclosure. Accordingly, other implementations are within the scope of the following claims. For example, the actions recited in the claims can be performed in a different order and still achieve desirable results.

The terminology used herein is for the purpose of describing particular exemplary configurations only and is not intended to be limiting. As used herein, the singular articles “a,” “an,” and “the” may be intended to include the plural forms as well, unless the context clearly indicates otherwise. The terms “comprises,” “comprising,” “including,” and “having,” are inclusive and therefore specify the presence of features, steps, operations, elements, and/or components, but do not preclude the presence or addition of one or more other features, steps, operations, elements, components, and/or groups thereof. The method steps, processes, and operations described herein are not to be construed as necessarily requiring their performance in the particular order discussed or illustrated, unless specifically identified as an order of performance. Additional or alternative steps may be employed.

When an element or layer is referred to as being “on,” “engaged to,” “connected to,” “attached to,” or “coupled to” another element or layer, it may be directly on, engaged, connected, attached, or coupled to the other element or layer, or intervening elements or layers may be present. In contrast, when an element is referred to as being “directly on,” “directly engaged to,” “directly connected to,” “directly attached to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The terms first, second, third, etc. may be used herein to describe various elements, components, regions, layers and/or sections. These elements, components, regions, layers and/or sections should not be limited by these terms. These terms may be only used to distinguish one element, component, region, layer or section from another region, layer or section. Terms such as “first,” “second,” and other numerical terms do not imply a sequence or order unless clearly indicated by the context. Thus, a first element, component, region, layer or section discussed below could be termed a second element, component, region, layer or section without departing from the teachings of the example configurations.

What is claimed is:

1. A hypodermic interface assembly comprising:
a hub;
a cannula;
a core member; and
a cannula carrier,
wherein the core member is non-removably connected to the cannula,
wherein the cannula carrier is non-removably connected to the core member, and
wherein the cannula carrier is controllably separable from the hub.

2. The hypodermic interface assembly of claim 1, wherein the core member has a body.

3. The hypodermic interface assembly of claim 2, wherein the body is defined by a proximal end surface, a distal end surface, and an outer surface extending between the proximal end surface and the distal end surface, wherein the body includes a cannula-receiving-passage defined by an inner surface that extends through the body of the core member.

4. The hypodermic interface assembly of claim 3, wherein the distal end surface of the body of the core member extends distally beyond a distal end surface of the cannula carrier.

5. The hypodermic interface assembly of claim 3, wherein the core member includes a cannula-carrier-engaging portion and wherein the cannula carrier is configured to receive the cannula-carrier-engaging portion.

6. The hypodermic interface assembly of claim 5, wherein the cannula-carrier-engaging portion of the core member is non-removably connected to the cannula carrier.

7. The hypodermic interface assembly of claim 6, wherein the cannula-carrier-engaging portion of the core member is friction-fit connected to the cannula carrier.

8. The hypodermic interface assembly of claim 3, wherein the cannula is disposed within:
a hub passage extending through the hub;
the cannula-receiving-passage of the core member; and
a cannula carrier passage extending through the cannula carrier.

9. The hypodermic interface assembly of claim 8, wherein a first portion of an outer surface of the cannula is arranged in a spaced-apart relationship with respect to a surface portion that defines the cannula carrier passage of the cannula carrier, and wherein a second portion of the outer surface of the cannula is disposed adjacent and non-removably-connected to a surface portion defining the cannula-receiving-passage of the core member.

10. The hypodermic interface assembly of claim 1, wherein the cannula carrier includes a head portion and at least one leg portion, and wherein a proximal end surface of the at least one leg portion is disposed adjacent an outer shoulder surface portion of an outer surface of the hub.

11. The hypodermic interface assembly of claim 1,
wherein the hub is defined by a distal end surface;
wherein the core member is defined by a body having a proximal end surface, and
wherein a portion of the cannula has a length that extends between the distal end surface of the hub and the proximal end surface of the core member and defines a hub-core distance, and
wherein the hub-core distance is configured to permit the cannula to bend or break at a region bound by the distal end surface of the hub and the proximal end surface of the core member when the hypodermic interface assembly is subjected to one or more radial forces.

12. The hypodermic interface assembly of claim 1, wherein the cannula is arranged relative the hub and the core member such that a first portion of the cannula is arranged within the hub, a second portion of the cannula extends between a distal end surface of the hub and a proximal end surface of the core member, a third portion of the cannula is arranged within the core member, and a fourth portion of the cannula extends beyond a distal end surface of the core member.

13. The hypodermic interface assembly of claim 12, wherein the second portion of the cannula is configured to permit the cannula to bend or break at a region bound by the second portion of the cannula when the hypodermic interface assembly is subjected to one or more radial forces.

14. A subassembly of a hypodermic interface assembly, the subassembly comprising:
a hub having a distal end surface;
a cannula having an outer surface that is non-removably connected to the hub, wherein the cannula extends distally away from the distal end surface of the hub; and
a core member having a body defined by a proximal end surface and a distal end surface, wherein the proximal end surface of the body is arranged at a hub-core distance away from a distal end surface of the hub.

15. The subassembly of claim 14, wherein the body includes an inner surface defining a cannula-receiving-passage that contains a portion of a length of the cannula, wherein the cannula-receiving-passage defines a passage diameter, wherein the inner surface is arrangeable in one of an inner surface preconfigured state and an inner surface deformed state.

16. The subassembly of claim 14, wherein when the inner surface is arranged in the inner surface preconfigured state, the cannula-receiving-passage is a cannula-receiving-passage and the passage diameter is greater than an outer diameter of the cannula such that the outer surface of the cannula is not joined to the inner surface of the body of the core member.

17. The subassembly of claim 16, wherein when the inner surface is arranged in the inner surface deformed state, the cannula-receiving-passage is a cannula-receiving-passage such that the passage diameter is deformed to define a deformed diameter that is approximately the same as but slightly greater than the outer diameter of the cannula whereby the outer surface of the cannula is non-removably connected to a deformed inner surface of the inner surface extending along at least a portion of the length of the body of the core member and defines the deformed diameter.

18. The subassembly of claim 17, wherein the passage diameter of the cannula-receiving-passage is constant along the length of the body of the core member, wherein the deformed inner surface of the inner surface is a portion of the inner surface of the body of the core member whereby the deformed diameter defines the passage diameter to be non-constant along the length of the body of the core member.

19. The subassembly of claim 15, wherein the body of the core member includes an outer surface arrangeable in one of:
an outer surface preconfigured state whereby the outer surface defines a preconfigured dimension; and
an outer surface deformed state whereby the outer surface defines a preconfigured-and-subsequently-deformed dimension whereby the portion of the length of the body of the core member defines a deformed outer surface of the outer surface that defines the preconfigured-and-subsequently-deformed dimension to be different from the preconfigured dimension.

20. The subassembly of claim 17, wherein proximal end surface defines a proximal opening of the body that permits proximal access to the cannula-receiving-passage, wherein the distal end surface defines a distal opening of the body that permits distal access to the cannula-receiving-passage.

21. The subassembly of claim 20, whereby a proximal portion of the cannula extends through the proximal opening of the body, wherein a distal portion of the cannula extends through the proximal opening of the body.
